SEQUENCE LISTING (308 sequences)

SEQ ID NO: 1 - DNA, 375 bp, Artificial Sequence
  Synthetic
caggtccagttggtacagtctggggctgacgtgaagaagcctggggcctcagtgaaggtctcctgcaaggtttccggacatatcctcactgatttatccatgcactgggtgcgacagcctcctggaaaaggacttgagtggatggcaggttttgatcctgaagaaggtaaaataatctacgcacagaagttccagggcagagtcaccatgaccgaggacacatctacagacacagcctacatggagctgagcagcctgagatctggggacacggccgtttattactgtgcaacaagcgatattttgactgggtattatagagactactacggtttggacgtctggggccaagggaccacgctcaccgtctcctca

SEQ ID NO: 2 - protein, 125 aa, Artificial Sequence
  Synthetic
QVQLVQSGADVKKPGASVKVSCKVSGHILTDLSMHWVRQPPGKGLEWMAGFDPEEGKIIYAQKFQGRVTMTEDTSTDTAYMELSSLRSGDTAVYYCATSDILTGYYRDYYGLDVWGQGTTLTVSS

SEQ ID NO: 3 - DNA, 24 bp, Artificial Sequence
  Synthetic
ggacatatcctcactgatttatcc

SEQ ID NO: 4 - protein, 8 aa, Artificial Sequence
  Synthetic
GHILTDLS

SEQ ID NO: 5 - DNA, 24 bp, Artificial Sequence
  Synthetic
tttgatcctgaagaaggtaaaata

SEQ ID NO: 6 - protein, 8 aa, Artificial Sequence
  Synthetic
FDPEEGKI

SEQ ID NO: 7 - DNA, 54 bp, Artificial Sequence
  Synthetic
gcaacaagcgatattttgactgggtattatagagactactacggtttggacgtc

SEQ ID NO: 8 - protein, 18 aa, Artificial Sequence
  Synthetic
ATSDILTGYYRDYYGLDV

SEQ ID NO: 9 - DNA, 336 bp, Artificial Sequence
  Synthetic
gatattgtgatgactcagtctccactcttcctgcccgtcacccctggagagccggcctccatctcctgcaggtctagtcagagcctcctgcatagtaaaggatacaactatttggattggtacctgcagaagccagggcagtctccacaactcctgatctatttgggttctaatcgggcctccggggtccctgacaggttcagtggcagtggatcaggcacagattttacactgaaaatcagcagagtggaggctgaagatgttggggtttattactgcatgcaaactctacaaactcctcggacgttcggccaagggaccaaggtggaaatcaaa

SEQ ID NO: 10 - protein, 112 aa, Artificial Sequence
  Synthetic
DIVMTQSPLFLPVTPGEPASISCRSSQSLLHSKGYNYLDWYLQKPGQSPQLLIYLGSNRASGVPDRFSGSGSGTDFTLKISRVEAEDVGVYYCMQTLQTPRTFGQGTKVEIK

SEQ ID NO: 11 - DNA, 33 bp, Artificial Sequence
  Synthetic
cagagcctcctgcatagtaaaggatacaactat

SEQ ID NO: 12 - protein, 11 aa, Artificial Sequence
  Synthetic
QSLLHSKGYNY

SEQ ID NO: 13 - DNA, 9 bp, Artificial Sequence
  Synthetic
ttgggttct

SEQ ID NO: 14 - protein, 3 aa, Artificial Sequence
  Synthetic
LGS

SEQ ID NO: 15 - DNA, 27 bp, Artificial Sequence
  Synthetic
atgcaaactctacaaactcctcggacg

SEQ ID NO: 16 - protein, 9 aa, Artificial Sequence
  Synthetic
MQTLQTPRT

SEQ ID NO: 17 - DNA, 375 bp, Artificial Sequence
  Synthetic
caggtccagttggtacagtctggggctgacgtgaagaagcctggggcctcagtgaaggtctcctgcaaggtttccggacatatcctcactgatttatccatgcactgggtgcgacaggctcctggaaaagggcttgagtggatgggaggttttgatcctgaagaaggtgaaataatctacgcacagaagttccagggcagagtcaccatgaccgaggacacatctacagacacagcctacatggagctgagcagcctgagatctggggacacggccgtttattactgtgcaacaagcgatattttgactggttattatagagactactacggtttggacgtctggggccaagggaccacgctcaccgtctcctca

SEQ ID NO: 18 - protein, 125 aa, Artificial Sequence
  Synthetic
QVQLVQSGADVKKPGASVKVSCKVSGHILTDLSMHWVRQAPGKGLEWMGGFDPEEGEIIYAQKFQGRVTMTEDTSTDTAYMELSSLRSGDTAVYYCATSDILTGYYRDYYGLDVWGQGTTLTVSS

SEQ ID NO: 19 - DNA, 24 bp, Artificial Sequence
  Synthetic
ggacatatcctcactgatttatcc

SEQ ID NO: 20 - protein, 8 aa, Artificial Sequence
  Synthetic
GHILTDLS

SEQ ID NO: 21 - DNA, 24 bp, Artificial Sequence
  Synthetic
tttgatcctgaagaaggtgaaata

SEQ ID NO: 22 - protein, 8 aa, Artificial Sequence
  Synthetic
FDPEEGEI

SEQ ID NO: 23 - DNA, 54 bp, Artificial Sequence
  Synthetic
gcaacaagcgatattttgactggttattatagagactactacggtttggacgtc

SEQ ID NO: 24 - protein, 18 aa, Artificial Sequence
  Synthetic
ATSDILTGYYRDYYGLDV

SEQ ID NO: 25 - DNA, 336 bp, Artificial Sequence; identical to SEQ ID NO: 9
  Synthetic

SEQ ID NO: 26 - protein, 112 aa, Artificial Sequence; identical to SEQ ID NO: 10
  Synthetic

SEQ ID NO: 27 - DNA, 33 bp, Artificial Sequence; identical to SEQ ID NO: 11
  Synthetic

SEQ ID NO: 28 - protein, 11 aa, Artificial Sequence
  Synthetic
QSLLHSKGYNY

SEQ ID NO: 29 - DNA, 9 bp, Artificial Sequence
  Synthetic
ttgggttct

SEQ ID NO: 30 - protein, 3 aa, Artificial Sequence
  Synthetic
LGS

SEQ ID NO: 31 - DNA, 27 bp, Artificial Sequence
  Synthetic
atgcaaactctacaaactcctcggacg

SEQ ID NO: 32 - protein, 9 aa, Artificial Sequence
  Synthetic
MQTLQTPRT

SEQ ID NO: 33 - DNA, 372 bp, Artificial Sequence
  Synthetic
gaggagcaactggtggagtctgggggagacttggtacagcctggagggtccctaagactctcctgtgcagcctctggattcactctcagtagttatgaaatgaactgggtccgccaggctccagggaaggggctggagtgggtttcatacattagtagaggtggtagtctgatacactacacagactctgtgaagggccgattcaccatctccagagacaacgccaagaattcactgtatctgcaaatgaacagcctgagagccgaggacacggctgtttattactgtgtgagagacccagcagctcgttatcattattattatcacggtatggacgtctggggccaagggaccacggtcaccgtctcctca

SEQ ID NO: 34 - protein, 124 aa, Artificial Sequence
  Synthetic
EEQLVESGGDLVQPGGSLRLSCAASGFTLSSYEMNWVRQAPGKGLEWVSYISRGGSLIHYTDSVKGRFTISRDNAKNSLYLQMNSLRAEDTAVYYCVRDPAARYHYYYHGMDVWGQGTTVTVSS

SEQ ID NO: 35 - DNA, 24 bp, Artificial Sequence
  Synthetic
ggattcactctcagtagttatgaa

SEQ ID NO: 36 - protein, 8 aa, Artificial Sequence
  Synthetic
GFTLSSYE

SEQ ID NO: 37 - DNA, 24 bp, Artificial Sequence
  Synthetic
attagtagaggtggtagtctgata

SEQ ID NO: 38 - protein, 8 aa, Artificial Sequence
  Synthetic
ISRGGSLI

SEQ ID NO: 39 - DNA, 51 bp, Artificial Sequence
  Synthetic
gtgagagacccagcagctcgttatcattattattatcacggtatggacgtc

SEQ ID NO: 40 - protein, 17 aa, Artificial Sequence
  Synthetic
VRDPAARYHYYYHGMDV

SEQ ID NO: 41 - DNA, 336 bp, Artificial Sequence
  Synthetic
gatattgtgatgactcagtctccactctccctgcccgtcacccctggagagccggcctccatctcctgcaggtctagtcagagcctcctgcacaataatggatataactatttggattggtatctgcagaagccagggcagtctccacagctcctgatctatttgggttctagtcgggcctccggggtccctgacaggttcagtggcagtggatcaggcacagattttatactgaaaatcagcagagtggaggctgaagatgttggggtttattactgcatgcaagctctacaaactccgtggacgttcggccgagggaccaaggtggaaatcaaa

SEQ ID NO: 42 - protein, 112 aa, Artificial Sequence
  Synthetic
DIVMTQSPLSLPVTPGEPASISCRSSQSLLHNNGYNYLDWYLQKPGQSPQLLIYLGSSRASGVPDRFSGSGSGTDFILKISRVEAEDVGVYYCMQALQTPWTFGRGTKVEIK

SEQ ID NO: 43 - DNA, 33 bp, Artificial Sequence
  Synthetic
cagagcctcctgcacaataatggatataactat

SEQ ID NO: 44 - protein, 11 aa, Artificial Sequence
  Synthetic
QSLLHNNGYNY

SEQ ID NO: 45 - DNA, 9 bp, Artificial Sequence
  Synthetic
ttgggttct

SEQ ID NO: 46 - protein, 3 aa, Artificial Sequence
  Synthetic
LGS

SEQ ID NO: 47 - DNA, 27 bp, Artificial Sequence
  Synthetic
atgcaagctctacaaactccgtggacg

SEQ ID NO: 48 - protein, 9 aa, Artificial Sequence
  Synthetic
MQALQTPWT

SEQ ID NO: 49 - DNA, 384 bp, Artificial Sequence
  Synthetic
gaggtgcagctggtggagtctgggggaggcgtggtccagcctgggaggtccctgagactctcctgtgcagcctctggattcaccttcagtagttatgacatgcactgggtccgccaggctccaggcaaggggctggagtgggtggcagttatatcatctgatggacgtgataaatactatgtagactccgtgaagggccgattcaccatctccagagacaactccaagaacacgctttatctgcaaatgaacagcctgagagctgaggacacggctgtttattactgtgcgaaagagatggtgtattacgatattttgactggttatcataactactacggtatggacgtctggggccaagggaccacggtcaccgtctcctca

SEQ ID NO: 50 - protein, 128 aa, Artificial Sequence
  Synthetic
EVQLVESGGGVVQPGRSLRLSCAASGFTFSSYDMHWVRQAPGKGLEWVAVISSDGRDKYYVDSVKGRFTISRDNSKNTLYLQMNSLRAEDTAVYYCAKEMVYYDILTGYHNYYGMDVWGQGTTVTVSS

SEQ ID NO: 51 - DNA, 24 bp, Artificial Sequence
  Synthetic
ggattcaccttcagtagttatgac

SEQ ID NO: 52 - protein, 8 aa, Artificial Sequence
  Synthetic
GFTFSSYD

SEQ ID NO: 53 - DNA, 24 bp, Artificial Sequence
  Synthetic
atatcatctgatggacgtgataaa

SEQ ID NO: 54 - protein, 8 aa, Artificial Sequence
  Synthetic
ISSDGRDK

SEQ ID NO: 55 - DNA, 63 bp, Artificial Sequence
  Synthetic
gcgaaagagatggtgtattacgatattttgactggttatcataactactacggtatggacgtc

SEQ ID NO: 56 - protein, 21 aa, Artificial Sequence
  Synthetic
AKEMVYYDILTGYHNYYGMDV

SEQ ID NO: 57 - DNA, 324 bp, Artificial Sequence
  Synthetic
gacatcgtgatgacccagtctccatcctcactgtctgcatctgtaggagacagagtcaccatcacttgtcgggcgagtcagggcattaacaattatttagcctggtttcagcagaaaccagggaaagcccctaagtccctgatccatactgcatccagtttgcaaagtggggtcccatcaaagttcagcggcagtggatctgggacagatttcactctcaccatcagcagcctgcagcctgaagattttgcaacttattactgccaacagtataatacttaccctctcactttcggcggagggaccaaagtggagatcaaacga

SEQ ID NO: 58 - protein, 108 aa, Artificial Sequence
  Synthetic
DIVMTQSPSSLSASVGDRVTITCRASQGINNYLAWFQQKPGKAPKSLIHTASSLQSGVPSKFSGSGSGTDFTLTISSLQPEDFATYYCQQYNTYPLTFGGGTKVEIKR

SEQ ID NO: 59 - DNA, 18 bp, Artificial Sequence
  Synthetic
cagggcattaacaattat

SEQ ID NO: 60 - protein, 6 aa, Artificial Sequence
  Synthetic
QGINNY

SEQ ID NO: 61 - DNA, 9 bp, Artificial Sequence
  Synthetic
actgcatcc

SEQ ID NO: 62 - protein, 3 aa, Artificial Sequence
  Synthetic
TAS

SEQ ID NO: 63 - DNA, 27 bp, Artificial Sequence
  Synthetic
caacagtataatacttaccctctcact

SEQ ID NO: 64 - protein, 9 aa, Artificial Sequence
  Synthetic
QQYNTYPLT

SEQ ID NO: 65 - DNA, 381 bp, Artificial Sequence
  Synthetic
caggtgcagctggtggagtctgggggaggcgtggtccagcctgggaggtccctgagactctcctgtgcagcctctggattcaccttcagtagttatgacatgcactgggtccgccaggctccaggcaaggggctggagtgggtggcagttatatcatctgatggacgtgataaatactatgtagactccgtgaagggccgattcaccatctccagagacaactccaagaacacgctttatctgcaaatgaacagcctgagagctgaggacacggctgtttattactgtgcgaaagagatggtgtattacgatattttgactggttatcataactactacggtatggacgtctggggccaagggaccacggtcaccgtctcc

SEQ ID NO: 66 - protein, 127 aa, Artificial Sequence
  Synthetic
QVQLVESGGGVVQPGRSLRLSCAASGFTFSSYDMHWVRQAPGKGLEWVAVISSDGRDKYYVDSVKGRFTISRDNSKNTLYLQMNSLRAEDTAVYYCAKEMVYYDILTGYHNYYGMDVWGQGTTVTVS

SEQ ID NO: 67 - DNA, 321 bp, Artificial Sequence
  Synthetic
gacatccagatgacccagtctccatcctcactgtctgcatctgtaggagacagagtcaccatcacttgtcgggcgagtcagggcattaacaattatttagcctggtttcagcagaaaccagggaaagcccctaagtccctgatccatactgcatccagtttgcaaagtggggtcccatcaaagttcagcggcagtggatctgggacagatttcactctcaccatcagcagcctgcagcctgaagattttgcaacttattactgccaacagtataatacttaccctctcactttcggcggagggaccaaggtggagatcaaa

SEQ ID NO: 68 - protein, 107 aa, Artificial Sequence
  Synthetic
DIQMTQSPSSLSASVGDRVTITCRASQGINNYLAWFQQKPGKAPKSLIHTASSLQSGVPSKFSGSGSGTDFTLTISSLQPEDFATYYCQQYNTYPLTFGGGTKVEIK

SEQ ID NO: 69 - DNA, 384 bp, Artificial Sequence
  Synthetic
caggtgcagctggtgcagtctgggggaggcttggtccagcctggggggtccctgagactctcctgtgcagcctccggattcacctttagtaactatttgatgaactgggtccgccaggctccagggaaggggctggagtggctggccaacatacaggaagatggaattgagaaatactatgtggactctgtgaagggccgattcaccatctccagagacaacgccaagaactcactgtatctgcaaatgaacagcctgagagccgaggacacggctgtgtattactgtgcgagagagccctcccattacgatattttgactggttatgactactattacggtatggacgtctggggccaagggaccacggtcaccgtctcctca

SEQ ID NO: 70 - protein, 128 aa, Artificial Sequence
  Synthetic
QVQLVQSGGGLVQPGGSLRLSCAASGFTFSNYLMNWVRQAPGKGLEWLANIQEDGIEKYYVDSVKGRFTISRDNAKNSLYLQMNSLRAEDTAVYYCAREPSHYDILTGYDYYYGMDVWGQGTTVTVSS

SEQ ID NO: 71 - DNA, 24 bp, Artificial Sequence
  Synthetic
ggattcacctttagtaactatttg

SEQ ID NO: 72 - protein, 8 aa, Artificial Sequence
  Synthetic
GFTFSNYL

SEQ ID NO: 73 - DNA, 24 bp, Artificial Sequence
  Synthetic
atacaggaagatggaattgagaaa

SEQ ID NO: 74 - protein, 8 aa, Artificial Sequence
  Synthetic
IQEDGIEK

SEQ ID NO: 75 - DNA, 63 bp, Artificial Sequence
  Synthetic
gcgagagagccctcccattacgatattttgactggttatgactactattacggtatggacgtc

SEQ ID NO: 76 - protein, 21 aa, Artificial Sequence
  Synthetic
AREPSHYDILTGYDYYYGMDV

SEQ ID NO: 77 - DNA, 324 bp, Artificial Sequence
  Synthetic
gacatccagttgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcattctcacagtcagcagcctgcagcctgaagactttgcaacttattactgtctacagtataatagtaacccattcactttcggccctgggaccaaggtggagatcaaacga

SEQ ID NO: 78 - protein, 108 aa, Artificial Sequence
  Synthetic
DIQLTQSPSSLSASVGDRVTITCRASQGIRNDLGWYQQKPGKAPKRLIYAASSLQSGVPSRFSGSGSGTEFILTVSSLQPEDFATYYCLQYNSNPFTFGPGTKVEIKR

SEQ ID NO: 79 - DNA, 18 bp, Artificial Sequence
  Synthetic
cagggcattagaaatgat

SEQ ID NO: 80 - protein, 6 aa, Artificial Sequence
  Synthetic
QGIRND

SEQ ID NO: 81 - DNA, 9 bp, Artificial Sequence
  Synthetic
gctgcatcc

SEQ ID NO: 82 - protein, 3 aa, Artificial Sequence
  Synthetic
AAS

SEQ ID NO: 83 - DNA, 27 bp, Artificial Sequence
  Synthetic
ctacagtataatagtaacccattcact

SEQ ID NO: 84 - protein, 9 aa, Artificial Sequence
  Synthetic
LQYNSNPFT

SEQ ID NO: 85 - DNA, 381 bp, Artificial Sequence
  Synthetic
gaggtgcagctggtggagtctgggggaggcttggtccagcctggggggtccctgagactctcctgtgcagcctccggattcacctttagtaactatttgatgaactgggtccgccaggctccagggaaggggctggagtggctggccaacatacaggaagatggaattgagaaatactatgtggactctgtgaagggccgattcaccatctccagagacaacgccaagaactcactgtatctgcaaatgaacagcctgagagccgaggacacggctgtgtattactgtgcgagagagccctcccattacgatattttgactggttatgactactattacggtatggacgtctggggccaagggaccacggtcaccgtctcc

SEQ ID NO: 86 - protein, 127 aa, Artificial Sequence
  Synthetic
EVQLVESGGGLVQPGGSLRLSCAASGFTFSNYLMNWVRQAPGKGLEWLANIQEDGIEKYYVDSVKGRFTISRDNAKNSLYLQMNSLRAEDTAVYYCAREPSHYDILTGYDYYYGMDVWGQGTTVTVS

SEQ ID NO: 87 - DNA, 321 bp, Artificial Sequence
  Synthetic
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcattctcacagtcagcagcctgcagcctgaagactttgcaacttattactgtctacagtataatagtaacccattcactttcggccctgggaccaaagtggatatcaaa

SEQ ID NO: 88 - protein, 107 aa, Artificial Sequence
  Synthetic
DIQMTQSPSSLSASVGDRVTITCRASQGIRNDLGWYQQKPGKAPKRLIYAASSLQSGVPSRFSGSGSGTEFILTVSSLQPEDFATYYCLQYNSNPFTFGPGTKVDIK

SEQ ID NO: 89 - DNA, 381 bp, Artificial Sequence
  Synthetic
gaggtgcagctggtgcagtctgggggagccttggtacagcctggggggtccctgagactctcctgtacagcctctggtttcaccttcagtaactacgacatgcactgggtccgccaaactacaggaaaaggtctggagtggatctcagctattgatactgctggtgacacatactatccaggctccgtgaagggccgattcaccgtctccagagaaaatgccaagaactccttttatcttcaaatgaacagcctgagagccggggacacggctgtgtattactgtgcaagggaggggaagtattacgatattttgactggtgactaccactactacggtatggacgtctggggccaagggaccacggtcaccgtctcctca

SEQ ID NO: 90 - protein, 127 aa, Artificial Sequence
  Synthetic
EVQLVQSGGALVQPGGSLRLSCTASGFTFSNYDMHWVRQTTGKGLEWISAIDTAGDTYYPGSVKGRFTVSRENAKNSFYLQMNSLRAGDTAVYYCAREGKYYDILTGDYHYYGMDVWGQGTTVTVSS

SEQ ID NO: 91 - DNA, 24 bp, Artificial Sequence
  Synthetic
ggtttcaccttcagtaactacgac

SEQ ID NO: 92 - protein, 8 aa, Artificial Sequence
  Synthetic
GFTFSNYD

SEQ ID NO: 93 - DNA, 21 bp, Artificial Sequence
  Synthetic
attgatactgctggtgacaca

SEQ ID NO: 94 - protein, 7 aa, Artificial Sequence
  Synthetic
IDTAGDT

SEQ ID NO: 95 - DNA, 63 bp, Artificial Sequence
  Synthetic
gcaagggaggggaagtattacgatattttgactggtgactaccactactacggtatggacgtc

SEQ ID NO: 96 - protein, 21 aa, Artificial Sequence
  Synthetic
AREGKYYDILTGDYHYYGMDV

SEQ ID NO: 97 - DNA, 324 bp, Artificial Sequence
  Synthetic
gccatccggatgacccagtctccatcctccctgtctgcatctgtaggagacagagtcaccatcacttgtcgggcaagtcagggcattagaaatgatttaggctggtatcagcagaaaccagggaaagcccctaagcgactgatctatgctacatccagtttgcaaagtggggtcccatcaaggttcagcggcagtggatctgggacagaattcactctcacaatcagcagcctgcagcctgaagattttgcaacttattactgtctacagcataatagttacccgctcactttcggcggagggaccaaggtggaaatcaaacga

SEQ ID NO: 98 - protein, 108 aa, Artificial Sequence
  Synthetic
AIRMTQSPSSLSASVGDRVTITCRASQGIRNDLGWYQQKPGKAPKRLIYATSSLQSGVPSRFSGSGSGTEFTLTISSLQPEDFATYYCLQHNSYPLTFGGGTKVEIKR

SEQ ID NO: 99 - DNA, 18 bp, Artificial Sequence
  Synthetic
cagggcattagaaatgat

SEQ ID NO: 100 - protein, 6 aa, Artificial Sequence
  Synthetic
QGIRND

SEQ ID NO: 101 - DNA, 9 bp, Artificial Sequence
  Synthetic
gctacatcc

SEQ ID NO: 102 - protein, 3 aa, Artificial Sequence
  Synthetic
ATS

SEQ ID NO: 103 - DNA, 27 bp, Artificial Sequence
  Synthetic
ctacagcataatagttacccgctcact

SEQ ID NO: 104 - protein, 9 aa, Artificial Sequence
  Synthetic
LQHNSYPLT

SEQ ID NO: 105 - DNA, 378 bp, Artificial Sequence
  Synthetic
gaggtgcagctggtggagtctgggggagccttggtacagcctggggggtccctgagactctcctgtacagcctctggtttcaccttcagtaactacgacatgcactgggtccgccaaactacaggaaaaggtctggagtggatctcagctattgatactgctggtgacacatactatccaggctccgtgaagggccgattcaccgtctccagagaaaatgccaagaactccttttatcttcaaatgaacagcctgagagccggggacacggctgtgtattactgtgcaagggaggggaagtattacgatattttgactggtgactaccactactacggtatggacgtctggggccaagggaccacggtcaccgtctcc

SEQ ID NO: 106 - protein, 126 aa, Artificial Sequence
  Synthetic
EVQLVESGGALVQPGGSLRLSCTASGFTFSNYDMHWVRQTTGKGLEWISAIDTAGDTYYPGSVKGRFTVSRENAKNSFYLQMNSLRAGDTAVYYCAREGKYYDILTGDYHYYGMDVWGQGTTVTVS

SEQ ID NO: 107 - DNA, 321 bp, Artificial Sequence
  Synthetic
gacatccagatgacccagtctccatcctccctgtctgcatctgtaggagacagagtcaccatcacttgtcgggcaagtcagggcattagaaatgatttaggctggtatcagcagaaaccagggaaagcccctaagcgactgatctatgctacatccagtttgcaaagtggggtcccatcaaggttcagcggcagtggatctgggacagaattcactctcacaatcagcagcctgcagcctgaagattttgcaacttattactgtctacagcataatagttacccgctcactttcggcggagggaccaagctggagatcaaa

SEQ ID NO: 108 - protein, 107 aa, Artificial Sequence
  Synthetic
DIQMTQSPSSLSASVGDRVTITCRASQGIRNDLGWYQQKPGKAPKRLIYATSSLQSGVPSRFSGSGSGTEFTLTISSLQPEDFATYYCLQHNSYPLTFGGGTKLEIK

SEQ ID NO: 109 - DNA, 372 bp, Artificial Sequence
  Synthetic
caggtgcagctggtgcagtctgggggaggcgtggtccagcctgggaggtccctgagactctcctgtgcagcgtctgggttcacctttagtaactttggcatgcactgggtccgccaggctccaggcaaggggctggagtgggtggcagttatatggtttgatgaaattgataaatactatgcagactccgtgaagggccgattcaccatctccagagacaattccaagaacacgctgtatccgcaaatgaacagcctgagagccgaagacacggctgtgtattactgtgcgcgagaagattacgatattttgactggttactattacgctatggacgtctggggccaagggaccacggtcaccgtctcctca

SEQ ID NO: 110 - protein, 124 aa, Artificial Sequence
  Synthetic
QVQLVQSGGGVVQPGRSLRLSCAASGFTFSNFGMHWVRQAPGKGLEWVAVIWFDEIDKYYADSVKGRFTISRDNSKNTLYPQMNSLRAEDTAVYYCAREDYDILTGYYYAMDVWGQGTTVTVSS

SEQ ID NO: 111 - DNA, 24 bp, Artificial Sequence
  Synthetic
gggttcacctttagtaactttggc

SEQ ID NO: 112 - protein, 8 aa, Artificial Sequence
  Synthetic
GFTFSNFG

SEQ ID NO: 113 - DNA, 24 bp, Artificial Sequence
  Synthetic
atatggtttgatgaaattgataaa

SEQ ID NO: 114 - protein, 8 aa, Artificial Sequence
  Synthetic
IWFDEIDK

SEQ ID NO: 115 - DNA, 51 bp, Artificial Sequence
  Synthetic
gcgcgagaagattacgatattttgactggttactattacgctatggacgtc

SEQ ID NO: 116 - protein, 17 aa, Artificial Sequence
  Synthetic
AREDYDILTGYYYAMDV

SEQ ID NO: 117 - DNA, 321 bp, Artificial Sequence
  Synthetic
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctaatctatgctgcatcccgtttgcaaagtggggtcccatcgaggttcagcggcagtggatctgggacagaattcactctcacaatcagcagcctgcagcctgaagattttggaacttattactgtctacagcataatagtcaccccaccttcggccaagggaccaaggtggagatcaaacga

SEQ ID NO: 118 - protein, 107 aa, Artificial Sequence
  Synthetic
DIQMTQSPSSLSASVGDRVTITCRASQGIRNDLGWYQQKPGKAPKRLIYAASRLQSGVPSRFSGSGSGTEFTLTISSLQPEDFGTYYCLQHNSHPTFGQGTKVEIKR

SEQ ID NO: 119 - DNA, 18 bp, Artificial Sequence
  Synthetic
cagggcattagaaatgat

SEQ ID NO: 120 - protein, 6 aa, Artificial Sequence
  Synthetic
QGIRND

SEQ ID NO: 121 - DNA, 9 bp, Artificial Sequence
  Synthetic
gctgcatcc

SEQ ID NO: 122 - protein, 3 aa, Artificial Sequence
  Synthetic
AAS

SEQ ID NO: 123 - DNA, 24 bp, Artificial Sequence
  Synthetic
ctacagcataatagtcaccccacc

SEQ ID NO: 124 - protein, 8 aa, Artificial Sequence
  Synthetic
LQHNSHPT

SEQ ID NO: 125 - DNA, 369 bp, Artificial Sequence
  Synthetic
caggtgcagctggtggagtctgggggaggcgtggtccagcctgggaggtccctgagactctcctgtgcagcgtctgggttcacctttagtaactttggcatgcactgggtccgccaggctccaggcaaggggctggagtgggtggcagttatatggtttgatgaaattgataaatactatgcagactccgtgaagggccgattcaccatctccagagacaattccaagaacacgctgtatccgcaaatgaacagcctgagagccgaagacacggctgtgtattactgtgcgcgagaagattacgatattttgactggttactattacgctatggacgtctggggccaagggaccacggtcaccgtctcc

SEQ ID NO: 126 - protein, 123 aa, Artificial Sequence
  Synthetic
QVQLVESGGGVVQPGRSLRLSCAASGFTFSNFGMHWVRQAPGKGLEWVAVIWFDEIDKYYADSVKGRFTISRDNSKNTLYPQMNSLRAEDTAVYYCAREDYDILTGYYYAMDVWGQGTTVTVS

SEQ ID NO: 127 - DNA, 318 bp, Artificial Sequence
  Synthetic
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctaatctatgctgcatcccgtttgcaaagtggggtcccatcgaggttcagcggcagtggatctgggacagaattcactctcacaatcagcagcctgcagcctgaagattttggaacttattactgtctacagcataatagtcaccccaccttcggccaagggaccaaggtggagatcaaa

SEQ ID NO: 128 - protein, 106 aa, Artificial Sequence
  Synthetic
DIQMTQSPSSLSASVGDRVTITCRASQGIKNDLGWYQQKPGKAPKRLIYAASRLQSGVPSRFSGSGSGTEFTLTISSLQPEDFGTYYCLQHNSHPTFGQGTKVEIK

SEQ ID NO: 129 - DNA, 381 bp, Artificial Sequence
  Synthetic
gaggtgcagctggtggagtcggggggaggcatggtacagcctggggggtccctgagactctcctgtgcagcctctggattcacctccagtaactacgacatgcactgggtccgccaagctacaggaaaaggtctggagtgggtctcaagtattgatactgctggggacacttactatccagactccgtgaagggccgctttatcatctccagagaaaatgccaaaaactccctgtatcttcaaatgaatagcctgagagccggggacacggctgtgtattactgtacaagggagccccgaaattacgaaattttgactggtcactaccactaccacggtatggacatctggggccaagggaccacggtcaccgtctcctca

SEQ ID NO: 130 - protein, 127 aa, Artificial Sequence
  Synthetic
EVQLVESGGGMVQPGGSLRLSCAASGFTSSNYDMHWVRQATGKGLEWVSSIDTAGDTYYPDSVKGRFIISRENAKNSLYLQMNSLRAGDTAVYYCTREPRNYEILTGHYHYHGMDIWGQGTTVTVSS

SEQ ID NO: 131 - DNA, 24 bp, Artificial Sequence
  Synthetic
ggattcacctccagtaactacgac

SEQ ID NO: 132 - protein, 8 aa, Artificial Sequence
  Synthetic
GFTSSNYD

SEQ ID NO: 133 - DNA, 21 bp, Artificial Sequence
  Synthetic
attgatactgctggggacact

SEQ ID NO: 134 - protein, 7 aa, Artificial Sequence
  Synthetic
IDTAGDT

SEQ ID NO: 135 - DNA, 63 bp, Artificial Sequence
  Synthetic
acaagggagccccgaaattacgaaattttgactggtcactaccactaccacggtatggacatc

SEQ ID NO: 136 - protein, 21 aa, Artificial Sequence
  Synthetic
TREPRNYEILTGHYHYHGMDI

SEQ ID NO: 137 - DNA, 324 bp, Artificial Sequence
  Synthetic
gacatccagatgacccagtcgccatcctccctgtctgcatctgtaggagacagagtcaccatcacttgccgggcaagtcaggccattagaaatgatttaggctggtatcagcagaaaccagggaaagcccctaaactcctgatctatactgcattcagtttacagagtggggtcccatcaaggttcagcggcagtaaatctggcacagacttcactctcaccatcagcagcctgcagcctgaagattttgcgacttattactgtctgcaggattacactaatcctcggacgttcggccaagggaccaaggtggagatcaaacga

SEQ ID NO: 138 - protein, 108 aa, Artificial Sequence
  Synthetic
DIQMTQSPSSLSASVGDRVTITCRASQAIRNDLGWYQQKPGKAPKLLIYTAFSLQSGVPSRFSGSKSGTDFTLTISSLQPEDFATYYCLQDYTNPRTFGQGTKVEIKR

SEQ ID NO: 139 - DNA, 18 bp, Artificial Sequence
  Synthetic
caggccattagaaatgat

SEQ ID NO: 140 - protein, 6 aa, Artificial Sequence
  Synthetic
QAIRND

SEQ ID NO: 141 - DNA, 9 bp, Artificial Sequence
  Synthetic
actgcattc

SEQ ID NO: 142 - protein, 3 aa, Artificial Sequence
  Synthetic
TAF

SEQ ID NO: 143 - DNA, 27 bp, Artificial Sequence
  Synthetic
ctgcaggattacactaatcctcggacg

SEQ ID NO: 144 - protein, 9 aa, Artificial Sequence
  Synthetic
LQDYTNPRT

SEQ ID NO: 145 - DNA, 378 bp, Artificial Sequence
  Synthetic
gaggtgcagctggtggagtcggggggaggcatggtacagcctggggggtccctgagactctcctgtgcagcctctggattcacctccagtaactacgacatgcactgggtccgccaagctacaggaaaaggtctggagtgggtctcaagtattgatactgctggggacacttactatccagactccgtgaagggccgctttatcatctccagagaaaatgccaaaaactccctgtatcttcaaatgaatagcctgagagccggggacacggctgtgtattactgtacaagggagccccgaaattacgaaattttgactggtcactaccactaccacggtatggacatctggggccaagggaccacggtcaccgtctcc

SEQ ID NO: 146 - protein, 126 aa, Artificial Sequence
  Synthetic
EVQLVESGGGMVQPGGSLRLSCAASGFTSSNYDMHWVRQATGKGLEWVSSIDTAGDTYYPDSVKGRFIISRENAKNSLYLQMNSLRAGDTAVYYCTREPRNYEILTGHYHYHGMDIWGQGTTVTVS

SEQ ID NO: 147 - DNA, 321 bp, Artificial Sequence
  Synthetic
gccatccagatgacccagtcgccatcctccctgtctgcatctgtaggagacagagtcaccatcacttgccgggcaagtcaggccattagaaatgatttaggctggtatcagcagaaaccagggaaagcccctaaactcctgatctatactgcattcagtttacagagtggggtcccatcaaggttcagcggcagtaaatctggcacagacttcactctcaccatcagcagcctgcagcctgaagattttgcgacttattactgtctgcaggattacactaatcctcggacgttcggccaagggaccaaggtggaaatcaaa

SEQ ID NO: 148 - protein, 107 aa, Artificial Sequence
  Synthetic
AIQMTQSPSSLSASVGDRVTITCRASQAIRNDLGWYQQKPGKAPKLLIYTAFSLQSGVPSRFSGSKSGTDFTLTISSLQPEDFATYYCLQDYTNPRTFGQGTKVEIK

SEQ ID NO: 149 - DNA, 357 bp, Artificial Sequence
  synthetic
caggtgcagctggtggagtctgggggaggcgtggtccagcctgggaggtccctgagactctcctgtgcagcctctggattcgccttcagtaactatggcatgcactgggtccgccaggctccaggcaaggggctggaatgggtgacatttatatcatatgatggaagtaataaatactatgcagactccgtgaagggccgattcaccatctccagagacaattccaagaacacgctgtatctgcaagtgaacagcctgagagctgaggacacggctgtgtattactgtgcgaaagaagcagtattagctgccctctttgactactggggccagggaaccctggtcaccgtctcctca

SEQ ID NO: 150 - protein, 119 aa, Artificial Sequence
  synthetic
QVQLVESGGGVVQPGRSLRLSCAASGFAFSNYGMHWVRQAPGKGLEWVTFISYDGSNKYYADSVKGRFTISRDNSKNTLYLQVNSLRAEDTAVYYCAKEAVLAALFDYWGQGTLVTVSS

SEQ ID NO: 151 - DNA, 24 bp, Artificial Sequence
  synthetic
ggattcgccttcagtaactatggc

SEQ ID NO: 152 - protein, 8 aa, Artificial Sequence
  synthetic
GFAFSNYG

SEQ ID NO: 153 - DNA, 24 bp, Artificial Sequence
  synthetic
atatcatatgatggaagtaataaa

SEQ ID NO: 154 - protein, 8 aa, Artificial Sequence
  synthetic
ISYDGSNK

SEQ ID NO: 155 - DNA, 36 bp, Artificial Sequence
  synthetic
gcgaaagaagcagtattagctgccctctttgactac

SEQ ID NO: 156 - protein, 12 aa, Artificial Sequence
  synthetic
AKEAVLAALFDY

SEQ ID NO: 157 - DNA, 336 bp, Artificial Sequence
  synthetic
gacatcgtgatgacccagtctccagactccctggctgtgtctctgggcgagagggccaccatcaactgcaagtccagccagagtgttttatacagctccaacaatcagaactacttagcttggtaccagcagaaaccaggacagcctcctaagctgctcatttactgggcatctacccgggaatccggggtccctgaccgattcagtggcagcgggtctgggacagatttcactctcaccatcaacagcctgcaggctgaagatgtggcagtttattactgtcagcaatattatagtactcctacgttcggccaagggaccaaggtggaaatcaaa

SEQ ID NO: 158 - protein, 112 aa, Artificial Sequence
  synthetic
DIVMTQSPDSLAVSLGERATINCKSSQSVLYSSNNQNYLAWYQQKPGQPPKLLIYWASTRESGVPDRFSGSGSGTDFTLTINSLQAEDVAVYYCQQYYSTPTFGQGTKVEIK

SEQ ID NO: 159 - DNA, 36 bp, Artificial Sequence
  synthetic
cagagtgttttatacagctccaacaatcagaactac

SEQ ID NO: 160 - protein, 12 aa, Artificial Sequence
  synthetic
QSVLYSSNNQNY

SEQ ID NO: 161 - DNA, 9 bp, Artificial Sequence
  synthetic
tgggcatct

SEQ ID NO: 162 - protein, 3 aa, Artificial Sequence
  synthetic
WAS

SEQ ID NO: 163 - DNA, 24 bp, Artificial Sequence
  synthetic
cagcaatattatagtactcctacg

SEQ ID NO: 164 - protein, 8 aa, Artificial Sequence
  synthetic
QQYYSTPT

SEQ ID NO: 165 - DNA, 357 bp, Artificial Sequence
  synthetic
caggtgcagctggtggagtctgggggaggcgtggtccagcctgggaggtccctgagactctcctgtgcagcctctggattcacgttcaatacctatggcatgcactgggtccgccaggctccagtcaaggggctggagtgggtggcatttatatcaaatgataagagtaatacattctatgcagactccgtgaagggccgattcaccatctccagagacaattccaagaacacgctgtttctggaaatgaacagcctgacagctgaggacacggctgtttattactgtgcgaaagagtccattttagcagccctctttgactactggggccagggaaccctggtcactgtctcctca

SEQ ID NO: 166 - protein, 119 aa, Artificial Sequence
  synthetic
QVQLVESGGGVVQPGRSLRLSCAASGFTFNTYGMHWVRQAPVKGLEWVAFISNDKSNTFYADSVKGRFTISRDNSKNTLFLEMNSLTAEDTAVYYCAKESILAALFDYWGQGTLVTVSS

SEQ ID NO: 167 - DNA, 24 bp, Artificial Sequence
  synthetic
ggattcacgttcaatacctatggc

SEQ ID NO: 168 - protein, 8 aa, Artificial Sequence
  synthetic
GFTFNTYG

SEQ ID NO: 169 - DNA, 24 bp, Artificial Sequence
  synthetic
atatcaaatgataagagtaataca

SEQ ID NO: 170 - protein, 8 aa, Artificial Sequence
  synthetic
ISNDKSNT

SEQ ID NO: 171 - DNA, 36 bp, Artificial Sequence
  synthetic
gcgaaagagtccattttagcagccctctttgactac

SEQ ID NO: 172 - protein, 12 aa, Artificial Sequence
  synthetic
AKESILAALFDY

SEQ ID NO: 173 - DNA, 336 bp, Artificial Sequence
  synthetic
gacatcgtgatgacccagtctccagactccctggctgtgtctctgggcgagagggccaccatcaactgcaagtccagccagagtgttttatacagctccaacaataagaattacttagcttggtaccaacagaaaccaagacagcctcttaaactactcatttactgggcatctattcgggaatccggggtccctgaccgattcagtggcagcgggtctgggacagatttcactctcaccatcagcagcctgcaggctgaagatgtggcagtttattactgtcagcaattttatagtgttcccacttttggccaggggaccaagctggagatcaaa

SEQ ID NO: 174 - protein, 112 aa, Artificial Sequence
  synthetic
DIVMTQSPDSLAVSLGERATINCKSSQSVLYSSNNKNYLAWYQQKPRQPLKLLIYWASIRESGVPDRFSGSGSGTDFTLTISSLQAEDVAVYYCQQFYSVPTFGQGTKLEIK

SEQ ID NO: 175 - DNA, 36 bp, Artificial Sequence
  synthetic
cagagtgttttatacagctccaacaataagaattac

SEQ ID NO: 176 - protein, 12 aa, Artificial Sequence
  synthetic
QSVLYSSNNKNY

SEQ ID NO: 177 - DNA, 9 bp, Artificial Sequence
  synthetic
tgggcatct

SEQ ID NO: 178 - protein, 3 aa, Artificial Sequence
  synthetic
WAS

SEQ ID NO: 179 - DNA, 24 bp, Artificial Sequence
  synthetic
cagcaattttatagtgttcccact

SEQ ID NO: 180 - protein, 8 aa, Artificial Sequence
  synthetic
QQFYSVPT

SEQ ID NO: 181 - DNA, 357 bp, Artificial Sequence
  synthetic
caggtgcagctggtggagtctgggggaggcgtggtccagcctgggaggtccctgagactctcctgtgcagcctctggattcacgtttagtacctttggcatgcactgggtccgccaggctccagtcaaggggctggagtgggtggcttttatatcaaatgataagaataataaattctatgcagactccgtgaagggccgattcaccatctccagagacaattccagggacacgctatatctgcaaatgaacagcctgacacctgaggacacggctgtttattactgtgcgaaagagtccattttagcagccctctttgactactggggccagggaaccctggtcaccgtctcctca

SEQ ID NO: 182 - protein, 119 aa, Artificial Sequence
  synthetic
QVQLVESGGGVVQPGRSLRLSCAASGFTFSTFGMHWVRQAPVKGLEWVAFISNDKNNKFYADSVKGRFTISRDNSRDTLYLQMNSLTPEDTAVYYCAKESILAALFDYWGQGTLVTVSS

SEQ ID NO: 183 - DNA, 24 bp, Artificial Sequence
  synthetic
ggattcacgtttagtacctttggc

SEQ ID NO: 184 - protein, 8 aa, Artificial Sequence
  synthetic
GFTFSTFG

SEQ ID NO: 185 - DNA, 24 bp, Artificial Sequence
  synthetic
atatcaaatgataagaataataaa

SEQ ID NO: 186 - protein, 8 aa, Artificial Sequence
  synthetic
ISNDKNNK

SEQ ID NO: 187 - DNA, 36 bp, Artificial Sequence; identical to SEQ ID NO: 171
  synthetic

SEQ ID NO: 188 - protein, 12 aa, Artificial Sequence
  synthetic
AKESILAALFDY

SEQ ID NO: 189 - DNA, 336 bp, Artificial Sequence
  synthetic
gacatcgtgatgacccagtctccagactccctggctgtgtctctgggcgagagggccaccatcaactgcaagtccagccagagtgttttatacagctccaacaataaaaattacttagcttggtaccagcagaaaccaggacagcctcttaaacttctcatttactgggcatctattcgggaatccggggtccctgaccgattcagtggcagcgggtctgggacagatttcactctcaccatcagcagcctgcaggctgaagatgtggcagtttattactgtcagcaattttatactgttcccacttttggcctggggaccaagctggagatcaaa

SEQ ID NO: 190 - protein, 112 aa, Artificial Sequence
  synthetic
DIVMTQSPDSLAVSLGERATINCKSSQSVLYSSNNKNYLAWYQQKPGQPLKLLIYWASIRESGVPDRFSGSGSGTDFTLTISSLQAEDVAVYYCQQFYTVPTFGLGTKLEIK

SEQ ID NO: 191 - DNA, 36 bp, Artificial Sequence
  synthetic
cagagtgttttatacagctccaacaataaaaattac

SEQ ID NO: 192 - protein, 12 aa, Artificial Sequence
  synthetic
QSVLYSSNNKNY

SEQ ID NO: 193 - DNA, 9 bp, Artificial Sequence
  synthetic
tgggcatct

SEQ ID NO: 194 - protein, 3 aa, Artificial Sequence
  synthetic
WAS

SEQ ID NO: 195 - DNA, 24 bp, Artificial Sequence
  synthetic
cagcaattttatactgttcccact

SEQ ID NO: 196 - protein, 8 aa, Artificial Sequence
  synthetic
QQFYTVPT

SEQ ID NO: 197 - DNA, 357 bp, Artificial Sequence
  synthetic
caggtgcagctggtggagtctgggggaggcgtggtccagcctgggaggtccctgagactctcctgtgtagcctctggattcaccttcaggaactatgacatgcactgggtccgccaggctcctggcaaggggctggaatgggtggcagttacatcatctgatggacttaataaattctattcagactccgtgaagggccgattcaccatctccagagacaattccaagaacacgctgtctctgcaaattaccggcctgagagctgaggacacggctgtgtattactgtgcgaaagagtccattttagcagccctctttgactactggggccagggaaccctggtcaccgtctcctca

SEQ ID NO: 198 - protein, 119 aa, Artificial Sequence
  synthetic
QVQLVESGGGVVQPGRSLRLSCVASGFTFRNYDMHWVRQAPGKGLEWVAVTSSDGLNKFYSDSVKGRFTISRDNSKNTLSLQITGLRAEDTAVYYCAKESILAALFDYWGQGTLVTVSS

SEQ ID NO: 199 - DNA, 24 bp, Artificial Sequence
  synthetic
ggattcaccttcaggaactatgac

SEQ ID NO: 200 - protein, 8 aa, Artificial Sequence
  synthetic
GFTFRNYD

SEQ ID NO: 201 - DNA, 24 bp, Artificial Sequence
  synthetic
acatcatctgatggacttaataaa

SEQ ID NO: 202 - protein, 8 aa, Artificial Sequence
  synthetic
TSSDGLNK

SEQ ID NO: 203 - DNA, 36 bp, Artificial Sequence; identical to SEQ ID NO: 171
  synthetic

SEQ ID NO: 204 - protein, 12 aa, Artificial Sequence
  synthetic
AKESILAALFDY

SEQ ID NO: 205 - DNA, 336 bp, Artificial Sequence
  synthetic
gacatcgtgatgacccagtctccagactccctggctgtgtctctgggcgagagggccaccatcaactgcaagtccagccagagtgttttatacagctccaacaataagaactacttggcttggtaccagcagaaaccaggacagcctcctaagctgctcttttactgggcatctacccgggaatccggggtccctgaccgattcagtggcagcgggtctgggacagatttcactctcaccatcagcagcctgcaggctgaagatgtggcagtttattactgtcagcaacattatactactcccacttttggccaggggaccaagctggagatcaaa

SEQ ID NO: 206 - protein, 112 aa, Artificial Sequence
  synthetic
DIVMTQSPDSLAVSLGERATINCKSSQSVLYSSNNKNYLAWYQQKPGQPPKLLFYWASTRESGVPDRFSGSGSGTDFTLTISSLQAEDVAVYYCQQHYTTPTFGQGTKLEIK

SEQ ID NO: 207 - DNA, 36 bp, Artificial Sequence
  synthetic
cagagtgttttatacagctccaacaataagaactac

SEQ ID NO: 208 - protein, 12 aa, Artificial Sequence
  synthetic
QSVLYSSNNKNY

SEQ ID NO: 209 - DNA, 9 bp, Artificial Sequence
  synthetic
tgggcatct

SEQ ID NO: 210 - protein, 3 aa, Artificial Sequence
  synthetic
WAS

SEQ ID NO: 211 - DNA, 24 bp, Artificial Sequence
  synthetic
cagcaacattatactactcccact

SEQ ID NO: 212 - protein, 8 aa, Artificial Sequence
  synthetic
QQHYTTPT

SEQ ID NO: 213 - DNA, 357 bp, Artificial Sequence
  synthetic
caggtgcagctggtggagtctgggggaggcgtggtccagcctgggaggtccctgagactctcctgtgcagcctctggattcaccttcagtagctatggcatgcactgggtccgccagactccgggcaaggggctggagtgggtggcatttatatcatatgatggaaataataaatactatgcagactccgtgaagggccgattcaccatctccagagacaattccaagaacacgctgtttctgcaaatgaacagcctgagagctgaggacacggctgtgtattactgtgcgaaagagtccattttagcagccctctttgactactggggccagggaaccctggtcaccgtctcctca

SEQ ID NO: 214 - protein, 119 aa, Artificial Sequence
  synthetic
QVQLVESGGGVVQPGRSLRLSCAASGFTFSSYGMHWVRQTPGKGLEWVAFISYDGNNKYYADSVKGRFTISRDNSKNTLFLQMNSLRAEDTAVYYCAKESILAALFDYWGQGTLVTVSS

SEQ ID NO: 215 - DNA, 24 bp, Artificial Sequence
  synthetic
ggattcaccttcagtagctatggc

SEQ ID NO: 216 - protein, 8 aa, Artificial Sequence
  synthetic
GFTFSSYG

SEQ ID NO: 217 - DNA, 24 bp, Artificial Sequence
  synthetic
atatcatatgatggaaataataaa

SEQ ID NO: 218 - protein, 8 aa, Artificial Sequence
  synthetic
ISYDGNNK

SEQ ID NO: 219 - DNA, 36 bp, Artificial Sequence; identical to SEQ ID NO: 171
  synthetic

SEQ ID NO: 220 - protein, 12 aa, Artificial Sequence
  synthetic
AKESILAALFDY

SEQ ID NO: 221 - DNA, 336 bp, Artificial Sequence
  synthetic
gacatcgtgatgacccagtctccagactccctggctgtgtctctgggcgagagggccaccatcaactgcaagtccagccagagtgttttatacagctccaacaataagaactacttagcttggtaccagcagaaacctggacagcctcctaagctgctcatttactgggcatctacccgggaatccggggtccctgaccgattcagtggcagcgggtctgggacagatttcactctcaccatcagcagcctgcaggctgaagatgtggcactttattactgtcaacaatattataatactcccacttttggccaggggaccaagctggagatcaaa

SEQ ID NO: 222 - protein, 112 aa, Artificial Sequence
  synthetic
DIVMTQSPDSLAVSLGERATINCKSSQSVLYSSNNKNYLAWYQQKPGQPPKLLIYWASTRESGVPDRFSGSGSGTDFTLTISSLQAEDVALYYCQQYYNTPTFGQGTKLEIK

SEQ ID NO: 223 - DNA, 36 bp, Artificial Sequence; identical to SEQ ID NO: 207
  synthetic

SEQ ID NO: 224 - protein, 12 aa, Artificial Sequence
  synthetic
QSVLYSSNNKNY

SEQ ID NO: 225 - DNA, 9 bp, Artificial Sequence
  synthetic
tgggcatct

SEQ ID NO: 226 - protein, 3 aa, Artificial Sequence
  synthetic
WAS

SEQ ID NO: 227 - DNA, 24 bp, Artificial Sequence
  synthetic
caacaatattataatactcccact

SEQ ID NO: 228 - protein, 8 aa, Artificial Sequence
  synthetic
QQYYNTPT

SEQ ID NO: 229 - DNA, 357 bp, Artificial Sequence
  synthetic
caggtgcagctggtggagtctgggggaggcgtggtccagcctgggaggtccctgagactctcctgtgcagcctctggattcaccttcagtagctatggcatgcactgggtccgccaggctccagtcaaggggctggagtgggtggcatttatatcatatgatggaagtaataaatactatgcagactccgtgaagggccgattcaccatctccagagacaattccaagaacacgctgtatctccaaatgaacagcctgacagctgaggacacggctgtttattactgtgcgaaagagtccattttagcagccctctttgactactggggccagggaaccctggtcaccgtctcctca

SEQ ID NO: 230 - protein, 119 aa, Artificial Sequence
  synthetic
QVQLVESGGGVVQPGRSLRLSCAASGFTFSSYGMHWVRQAPVKGLEWVAFISYDGSNKYYADSVKGRFTISRDNSKNTLYLQMNSLTAEDTAVYYCAKESILAALFDYWGQGTLVTVSS

SEQ ID NO: 231 - DNA, 24 bp, Artificial Sequence
  synthetic
ggattcaccttcagtagctatggc

SEQ ID NO: 232 - protein, 8 aa, Artificial Sequence
  synthetic
GFTFSSYG

SEQ ID NO: 233 - DNA, 24 bp, Artificial Sequence
  synthetic
atatcatatgatggaagtaataaa

SEQ ID NO: 234 - protein, 8 aa, Artificial Sequence
  synthetic
ISYDGSNK

SEQ ID NO: 235 - DNA, 36 bp, Artificial Sequence; identical to SEQ ID NO: 171
  synthetic

SEQ ID NO: 236 - protein, 12 aa, Artificial Sequence
  synthetic
AKESILAALFDY

SEQ ID NO: 237 - DNA, 336 bp, Artificial Sequence
  synthetic
gacatcgtgatgacccagtctccagactccctggctgtgtctctgggcgagagggccaccatcaactgcaagtccagccagagtgttttatacagttccaacaataagaactacttagcttggtaccagcagaaaccaagacagcctcctaagctgctcatttactgggcatctattcgggaatccggggtccctgaccgattcagtggcagcgggtctgggacagatttcattctcaccatcagcagcctgcaggctgaagatgtggcagtttattactgtcagcaattttatagtattcccacttttggccaggggaccaagctggagatcaaa

SEQ ID NO: 238 - protein, 112 aa, Artificial Sequence
  synthetic
DIVMTQSPDSLAVSLGERATINCKSSQSVLYSSNNKNYLAWYQQKPRQPPKLLIYWASIRESGVPDRFSGSGSGTDFILTISSLQAEDVAVYYCQQFYSIPTFGQGTKLEIK

SEQ ID NO: 239 - DNA, 36 bp, Artificial Sequence
  synthetic
cagagtgttttatacagttccaacaataagaactac

SEQ ID NO: 240 - protein, 12 aa, Artificial Sequence
  synthetic
QSVLYSSNNKNY

SEQ ID NO: 241 - DNA, 9 bp, Artificial Sequence
  synthetic
tgggcatct

SEQ ID NO: 242 - protein, 3 aa, Artificial Sequence
  synthetic
WAS

SEQ ID NO: 243 - DNA, 24 bp, Artificial Sequence
  synthetic
cagcaattttatagtattcccact

SEQ ID NO: 244 - protein, 8 aa, Artificial Sequence
  synthetic
QQFYSIPT

SEQ ID NO: 245 - DNA, 357 bp, Artificial Sequence
  synthetic
caggtgcagctggtggagtctgggggaggcgtggtccagcctgggaggtccctgagactctcctgtgcagcctctggattcacctttagtagctatggcatgcactgggtccgccaggctccagtcaaggggctggagtgggtggcatttatatcaaatgataaaagtaataaatattatgcagactccttgaagggccgattcaccatctccagagacaattccaagaacacgctgtatctgcaaatgaacagcctgacagctgaagacacggctgtttattactgtgcgaaagagtccattttagcagccctctttgactattggggccagggaaccctggtcaccgtctcctca

SEQ ID NO: 246 - protein, 119 aa, Artificial Sequence
  synthetic
QVQLVESGGGVVQPGRSLRLSCAASGFTFSSYGMHWVRQAPVKGLEWVAFISNDKSNKYYADSLKGRFTISRDNSKNTLYLQMNSLTAEDTAVYYCAKESILAALFDYWGQGTLVTVSS

SEQ ID NO: 247 - DNA, 24 bp, Artificial Sequence
  synthetic
ggattcacctttagtagctatggc

SEQ ID NO: 248 - protein, 8 aa, Artificial Sequence
  synthetic
GFTFSSYG

SEQ ID NO: 249 - DNA, 24 bp, Artificial Sequence
  synthetic
atatcaaatgataaaagtaataaa

SEQ ID NO: 250 - protein, 8 aa, Artificial Sequence
  synthetic
ISNDKSNK

SEQ ID NO: 251 - DNA, 36 bp, Artificial Sequence
  synthetic
gcgaaagagtccattttagcagccctctttgactat

SEQ ID NO: 252 - protein, 12 aa, Artificial Sequence
  synthetic
AKESILAALFDY

SEQ ID NO: 253 - DNA, 336 bp, Artificial Sequence
  synthetic
gacatcgtgatgacccagtctccagactccctggctgtgtctctgggcgagagggccaccatcaactgcaagtccagccagagtgttttatacagctccaacaataagaactacttagcttggtaccagcagaaaccaagacagcctcctaagctactcatttactgggcatctattcgggaatccggggtccctgaccgattcagtggcagcgggtctgggacagatttcactctcaccatcagcagcctgcaggctgaagatgtggcagtttattactgtcaacaattttatagtgttcccacttttggccaggggaccaagctggagatcaaa

SEQ ID NO: 254 - protein, 112 aa, Artificial Sequence
  synthetic
DIVMTQSPDSLAVSLGERATINCKSSQSVLYSSNNKNYLAWYQQKPRQPPKLLIYWASIRESGVPDRFSGSGSGTDFTLTISSLQAEDVAVYYCQQFYSVPTFGQGTKLEIK

SEQ ID NO: 255 - DNA, 36 bp, Artificial Sequence; identical to SEQ ID NO: 207
  synthetic

SEQ ID NO: 256 - protein, 12 aa, Artificial Sequence
  synthetic
QSVLYSSNNKNY

SEQ ID NO: 257 - DNA, 9 bp, Artificial Sequence
  synthetic
tgggcatct

SEQ ID NO: 258 - protein, 3 aa, Artificial Sequence
  synthetic
WAS

SEQ ID NO: 259 - DNA, 24 bp, Artificial Sequence
  synthetic
caacaattttatagtgttcccact

SEQ ID NO: 260 - protein, 8 aa, Artificial Sequence
  synthetic
QQFYSVPT

SEQ ID NO: 261 - DNA, 357 bp, Artificial Sequence
  synthetic
caggtgcagctggtggagtctgggggaggcgtggtccagcctgggaggtccctgagactctcctgtgcagcctctggattcaccttcagtagctatggcatgcactgggtccgccaggctccagtcaaggggctggagtgggtggcatttatatcatttgatggaagtaataaatactatacagactccgtgaagggccgattcaccatctccagagacaattccaagaacacgctgtatctccaaatgaacagcctgacagctgaggacacggctatttattactgtgcgaaagagtccattttagcagccctctttgactactggggccagggaaccctggtcactgtctcctca

SEQ ID NO: 262 - protein, 119 aa, Artificial Sequence
  synthetic
QVQLVESGGGVVQPGRSLRLSCAASGFTFSSYGMHWVRQAPVKGLEWVAFISFDGSNKYYTDSVKGRFTISRDNSKNTLYLQMNSLTAEDTAIYYCAKESILAALFDYWGQGTLVTVSS

SEQ ID NO: 263 - DNA, 24 bp, Artificial Sequence
  synthetic
ggattcaccttcagtagctatggc

SEQ ID NO: 264 - protein, 8 aa, Artificial Sequence
  synthetic
GFTFSSYG

SEQ ID NO: 265 - DNA, 24 bp, Artificial Sequence
  synthetic
atatcatttgatggaagtaataaa

SEQ ID NO: 266 - protein, 8 aa, Artificial Sequence
  synthetic
ISFDGSNK

SEQ ID NO: 267 - DNA, 36 bp, Artificial Sequence; identical to SEQ ID NO: 171
  synthetic

SEQ ID NO: 268 - protein, 12 aa, Artificial Sequence
  synthetic
AKESILAALFDY

SEQ ID NO: 269 - DNA, 336 bp, Artificial Sequence
  synthetic
gacatcgtgatgacccagtctccagactccctggctgtgtctctgggcgagagggccaccatcaactgcaagtccagccagagtgttttatacagctccaacaataagaactacttagcttggtaccagcagaaaccaagacagcctcctaacctgctcatttactgggcatctattcgggaatccggggtccctgaccgattcagtggcagcgggtctgggacagatttcactctcaccatcagcagcctgcaggctgaagatgtggcattttattactgtcagcaattttatagtattcccacttttggccaggggaccaagctggagatcaaa

SEQ ID NO: 270 - protein, 112 aa, Artificial Sequence
  synthetic
DIVMTQSPDSLAVSLGERATINCKSSQSVLYSSNNKNYLAWYQQKPRQPPNLLIYWASIRESGVPDRFSGSGSGTDFTLTISSLQAEDVAFYYCQQFYSIPTFGQGTKLEIK

SEQ ID NO: 271 - DNA, 36 bp, Artificial Sequence; identical to SEQ ID NO: 207
  synthetic

SEQ ID NO: 272 - protein, 12 aa, Artificial Sequence
  synthetic
QSVLYSSNNKNY

SEQ ID NO: 273 - DNA, 9 bp, Artificial Sequence
  synthetic
tgggcatct

SEQ ID NO: 274 - protein, 3 aa, Artificial Sequence
  synthetic
WAS

SEQ ID NO: 275 - DNA, 24 bp, Artificial Sequence
  synthetic
cagcaattttatagtattcccact

SEQ ID NO: 276 - protein, 8 aa, Artificial Sequence
  synthetic
QQFYSIPT

SEQ ID NO: 277 - DNA, 357 bp, Artificial Sequence
  synthetic
caggtgcagctggtggagtctgggggaggcgtggtccagcctgggaggtccctgagactctcctgtgcagcctctggattcacctttaggacctatggcatgcactgggtccgccaggctccagtcaaggggctggagtgggtggcatttatatcaaaggatggaagtgataaatactatgtagactccgtgaagggccgattcaccatctccagagacaattccaagaacacgctgtttctgcaaatgaacagcctgacagctgaggacacggctgtttattattgtgcgaaagagtccattttagcagccctctttgactactggggccagggaaccctggtcactgtctcctca

SEQ ID NO: 278 - protein, 119 aa, Artificial Sequence
  synthetic
QVQLVESGGGVVQPGRSLRLSCAASGFTFRTYGMHWVRQAPVKGLEWVAFISKDGSDKYYVDSVKGRFTISRDNSKNTLFLQMNSLTAEDTAVYYCAKESILAALFDYWGQGTLVTVSS

SEQ ID NO: 279 - DNA, 24 bp, Artificial Sequence
  synthetic
ggattcacctttaggacctatggc

SEQ ID NO: 280 - protein, 8 aa, Artificial Sequence
  synthetic
GFTFRTYG

SEQ ID NO: 281 - DNA, 24 bp, Artificial Sequence
  synthetic
atatcaaaggatggaagtgataaa

SEQ ID NO: 282 - protein, 8 aa, Artificial Sequence
  synthetic
ISKDGSDK

SEQ ID NO: 283 - DNA, 36 bp, Artificial Sequence; identical to SEQ ID NO: 171
  synthetic

SEQ ID NO: 284 - protein, 12 aa, Artificial Sequence
  synthetic
AKESILAALFDY

SEQ ID NO: 285 - DNA, 336 bp, Artificial Sequence
  synthetic
gacatcgtgatgacccagtctccagactccctggctgtgtctctgggcgagagggccaccatcaactgcaagtccagccagagtgttttatacagctccaacaataagaactacttagcttggtaccagcagaaaccaagacagcctcctaaactcctcatttactgggcatctaatcgggaatccggggtccctgaccgattcagtggcagcgggtctgggacagatttcactctcaccatcagcagcctgcaggctgaagatgtggcagtttattactgtcagcaattttatagtgttcccacttttggccaggggaccaagctggagatcaaa

SEQ ID NO: 286 - protein, 112 aa, Artificial Sequence
  synthetic
DIVMTQSPDSLAVSLGERATINCKSSQSVLYSSNNKNYLAWYQQKPRQPPKLLIYWASNRESGVPDRFSGSGSGTDFTLTISSLQAEDVAVYYCQQFYSVPTFGQGTKLEIK

SEQ ID NO: 287 - DNA, 36 bp, Artificial Sequence; identical to SEQ ID NO: 207
  synthetic

SEQ ID NO: 288 - protein, 12 aa, Artificial Sequence
  synthetic
QSVLYSSNNKNY

SEQ ID NO: 289 - DNA, 9 bp, Artificial Sequence
  synthetic
tgggcatct

SEQ ID NO: 290 - protein, 3 aa, Artificial Sequence
  synthetic
WAS

SEQ ID NO: 291 - DNA, 24 bp, Artificial Sequence
  synthetic
cagcaattttatagtgttcccact

SEQ ID NO: 292 - protein, 8 aa, Artificial Sequence
  synthetic
QQFYSVPT

SEQ ID NO: 293 - DNA, 357 bp, Artificial Sequence
  synthetic
caggtgcagctggtggagtctgggggaggcgtggtccagcctgggaggtccctgagactctcctgtgcagcctctggattcacctttagtagctatggcatgcactgggtccgccaggctccagtcaaggggctggagtgggtggcatttatatcaaatgataaaagtaataaatactatgcagactccgtgaagggccgattcaccatctccagagacaattccaagaacacgctgtatctgcaaatgaacagcctgacagctgaggacacggctgtttattactgtgcgaaagagtccattttagcagccctctttgactcctggggccagggaaccctggtcaccgtctcctca

SEQ ID NO: 294 - protein, 119 aa, Artificial Sequence
  synthetic
QVQLVESGGGVVQPGRSLRLSCAASGFTFSSYGMHWVRQAPVKGLEWVAFISNDKSNKYYADSVKGRFTISRDNSKNTLYLQMNSLTAEDTAVYYCAKESILAALFDSWGQGTLVTVSS

SEQ ID NO: 295 - DNA, 24 bp, Artificial Sequence
  synthetic
ggattcacctttagtagctatggc

SEQ ID NO: 296 - protein, 8 aa, Artificial Sequence
  synthetic
GFTFSSYG

SEQ ID NO: 297 - DNA, 24 bp, Artificial Sequence
  synthetic
atatcaaatgataaaagtaataaa

SEQ ID NO: 298 - protein, 8 aa, Artificial Sequence
  synthetic
ISNDKSNK

SEQ ID NO: 299 - DNA, 36 bp, Artificial Sequence
  synthetic
gcgaaagagtccattttagcagccctctttgactcc

SEQ ID NO: 300 - protein, 12 aa, Artificial Sequence
  synthetic
AKESILAALFDS

SEQ ID NO: 301 - DNA, 336 bp, Artificial Sequence
  synthetic
gacatcgtgatgacccagtctccagactccctggctgtgtctctgggcgagagggccaccatcaactgcaagtccagccagagtgttttatacagctccaacaataagaactacttagcttggtaccagcagaaaccaagacagcctcctaagctgctcatttactgggcatctattcgggaatccggggtccctgaccgattcagtggcagcgggtctggggcagatttcactctcaccatcagcagcctgcaggctgcagatgtggcagtttattactgtcagcaattttatagtgttcccacttttggccaggggaccaagctggagatcaaa

SEQ ID NO: 302 - protein, 112 aa, Artificial Sequence
  synthetic
DIVMTQSPDSLAVSLGERATINCKSSQSVLYSSNNKNYLAWYQQKPRQPPKLLIYWASIRESGVPDRFSGSGSGADFTLTISSLQAADVAVYYCQQFYSVPTFGQGTKLEIK

SEQ ID NO: 303 - DNA, 36 bp, Artificial Sequence; identical to SEQ ID NO: 207
  synthetic

SEQ ID NO: 304 - protein, 12 aa, Artificial Sequence
  synthetic
QSVLYSSNNKNY

SEQ ID NO: 305 - DNA, 9 bp, Artificial Sequence
  synthetic
tgggcatct

SEQ ID NO: 306 - protein, 3 aa, Artificial Sequence
  synthetic
WAS

SEQ ID NO: 307 - DNA, 24 bp, Artificial Sequence
  synthetic
cagcaattttatagtgttcccact

SEQ ID NO: 308 - protein, 8 aa, Artificial Sequence
  synthetic
QQFYSVPT

(19) United States
(12) Patent Application Publication
(10) Pub. No.: US 2019/0248888 A1
(43) Pub. Date: Aug. 15, 2019

(54) METHODS OF LOWERING BLOOD GLUCOSE LEVELS

(71) Applicant: Regeneron Pharmaceuticals, Inc., Tarrytown, NY (US)

(72) Inventors: Jinrang Kim, New York, NY (US); Haruka Okamoto, New York, NY (US); Andrew J. Murphy, Croton-on-Hudson, NY (US); Jesper Gromada, Scarsdale, NY (US); George D. Yancopoulos, Yorktown Heights, NY (US)

(21) Appl. No.: 16/343,240

(22) PCT Filed: Oct. 19, 2017
(86) PCT No.: PCT/US17/57429

§ 371 Date: Apr. 18, 2019

Related U.S. Application Data
(60) Provisional application No. 62/410,763, filed on Oct. 20, 2016.

(51) Int. Cl. C07K 16/28; A61P 3/10; A61K 9/00; A61K 31/7088; A61K 31/366

(52) U.S. Cl. CPC C07K 16/28; A61P 3/10; A61K 31/366; A61K 31/7088; A61K 9/0019

(57) ABSTRACT

The present invention provides glucagon signaling pathway antagonists for use in combination with amino acid transport inhibitors, or mTOR inhibitors for lowering blood glucose levels in patients suffering from a disease or condition characterized in part by elevated blood glucose levels. According to certain embodiments of the invention, the glucagon signaling pathway antagonists are fully human antibodies that bind to human GCG or human GCGR. The antibodies of the invention, when combined with either SLC38A5 inhibitors or with an mTOR inhibitor are useful for lowering blood glucose levels, without resulting in alpha cell hyperplasia, and are also useful for the treatment of diseases and disorders associated with one or more GCGR biological activities, including the treatment of diabetes, and long-term complications associated with diabetes, or other metabolic disorders characterized in part by elevated blood glucose levels.

### FIELD OF THE INVENTION

The invention relates to methods of using a glucagon signaling pathway antagonist in combination with an amino acid transporter inhibitor to lower blood glucose levels in patients in need thereof, or to treat or to slow the progression of a disease or condition characterized in part by elevated blood glucose levels. More particularly, the invention relates to the use of a glucagon (GCG) inhibitor or a glucagon receptor (GCGR) antagonist antibody in combination with an inhibitor of SLC38A5 for lowering blood glucose levels in patients in need thereof.

### STATEMENT OF RELATED ART

Glucagon is a 29 residue polypeptide hormone, which in cooperation with insulin, mediates homeostatic regulation of the amount of glucose in the blood. Glucagon primarily acts by stimulating certain cells, for example, liver cells, to release glucose when blood glucose levels fall to maintain normal blood glucose levels. The action of glucagon is opposite to that of insulin, which stimulates cells to take up and store glucose whenever blood glucose levels rise. Glucagon is produced in the alpha cells of the pancreas, whereas insulin is secreted from the neighboring beta cells.

It is an imbalance of glucagon and insulin that may play an important role in several diseases, such as diabetes mellitus and diabetic ketoacidosis. In particular, studies have shown that higher basal glucagon levels and lack of suppression of postprandial glucagon secretion contribute to diabetic conditions in humans (Muller et al., N Eng J Med, 283: 109-115, 1970).

Glucagon responds to low blood glucose levels by stimulating hepatic glucose output. A key step in this process is that glucagon promotes the uptake and metabolism of amino acids in the liver. These amino acid metabolites are then used as substrates in the process of gluconeogenesis to produce glucose.

It is believed that glucagon's effects on elevating blood glucose levels are mediated in part by the activation of certain cellular pathways following the binding of glucagon (GCG) to its receptor (designated GCGR). GCGR is a member of the secretin subfamily (family B) of G-protein-coupled receptors and is predominantly expressed in the liver. The binding of glucagon to its receptor triggers a G-protein signal transduction cascade, activating intracellular cyclic AMP and leading to an increase in glucose output through de novo synthesis (gluconeogenesis) and glycogen breakdown (glycogenolysis) (Wakelam et al., Nature, (1986) 323:68-71; Unson et al., Peptides, (1989), 10:1171-1177; and Pittner and Fain, Biochem. J. (1991), 277:371-378).

The action of glucagon can be suppressed by providing an antagonist, such as a small molecule inhibitor, a GCG antibody, or a GCGR antibody, as described herein. Anti-GCG antibodies are mentioned, e.g., in U.S. Pat. Nos. 4,206,199; 4,221,777; 4,423,034; 4,272,433; 4,407,965; 5,712,105; and in PCT publications WO2007/124463 and WO2013/081993. Anti-GCGR antibodies are described in U.S. Pat. Nos. 5,770,445, 7,947,809, and 8,545,847; European patent application EP2074149A2; EP patent EP0658200B1; US patent publications 2009/0041784; 2009/0252727; and 2011/0223160; and PCT publication WO2008/036341. Small molecule inhibitors of GCG or GCGR are mentioned, e.g. in WO 07/47676; WO 06/86488; WO 05/123688; WO 05/121097; WO 06/14618; WO 08/42223; WO 08/98244; WO 2010/98948; US 20110306624; WO 2010/98994; WO 2010/88061; WO 2010/71750; WO 2010/30722; WO 06/104826; WO 05/65680; WO 06/102067; WO 06/17055; WO 2011/07722; or WO 09/140342.

Genetic disruption or pharmacologic inhibition of the hepatic glucagon pathway has invariably been shown to increase pancreatic alpha-cell mass. This has been observed in glucagon receptor knockout (Gcgr^{-/-}) mice (Gelling et al., 2003, PNAS 100:1438-1443), glucagon knockout mice (Hayashi et al., 2009, Mol. Endocrinol. 23:1990-1999), prohormone convertase 2 knockout mice (Webb et al., 2002, Diabetes 51:398-405), liver-specific Gcgr^{-/-} mice (Longuet et al., 2013, Diabetes 62:1196-1205) and liver-specific Gₛα knockout mice (Chen et al., 2005, J. Clin. Investigation 115:3217-3227). Pharmacologic knock-down of hepatic GCGR using antisense oligonucleotides (Sloop et al., 2004, J. Clin. Invest. 113:1571-1582; Liang et al., 2004, Diabetes 53:410-417) or administration of GCGR-blocking antibodies (Gu et al., 2009, J. Pharmacol. Exp Ther. 331:871-881; Okamoto et al., 2015, Endocrinology 156:2781-2794) also increases alpha-cell (also referred to herein as “a-cell”) mass in rodents. Furthermore, glucagon cell hyperplasia has been observed in patients with inactivating mutations in GCGR (Zhou et al., 2009, Pancreas 38:941-946; Sipos et al., 2015, J. Clin. Endocrinol Metab 5:E783-E788). Finally, a recent report showed that the GCGR inhibition-induced increase in plasma amino acids regulates alpha-cell hyperplasia in an mTOR-dependent manner (Solloway et al., 2015, Cell Reports 12:495-510).

There exists a need in the art for a method of lowering or normalizing blood glucose levels in patients suffering from a disease or disorder characterized in part by elevated blood glucose levels by administering a glucagon signaling pathway antagonist, such as a glucagon inhibitor or a glucagon receptor antagonist antibody as described herein, while at the same time preventing the alpha cell hyperplasia observed following administering such agents.

### BRIEF SUMMARY OF THE INVENTION

In its broadest aspect, the invention provides methods for lowering blood glucose levels in patients suffering from a disease or condition characterized in part by elevated blood glucose levels.

A first aspect of the invention provides a method of lowering blood glucose levels in patients suffering from a disease or condition characterized in part by elevated blood glucose levels by administering a therapeutically effective amount of a glucagon signaling pathway antagonist in combination with either a therapeutically effective amount of an inhibitor of an amino acid transporter, designated SLC38A5, or with a therapeutically effective amount of an inhibitor of mechanistic target of rapamycin (mTOR). The glucagon signaling pathway antagonist may be a small organic molecule, a protein, a peptide, an antibody or fragment thereof, an siRNA, or an antisense molecule that inhibits glucagon signaling by binding to either glucagon (GCG), or to the glucagon receptor (GCGR). The antagonist of glucagon signaling may also be an agent that inhibits production of glucagon.

In one embodiment, the glucagon signaling pathway antagonist is a fully human monoclonal antibody (mAb) or antigen-binding fragments thereof that bind specifically to the human glucagon receptor (hGCGR), to be used in combination with either a therapeutically effective amount of an inhibitor of an amino acid transporter, designated SLC38A5, or with a therapeutically effective amount of an inhibitor of mechanistic target of rapamycin (mTOR). The antibodies that bind specifically to the glucagon receptor inhibit or block its activity, for example, block the binding of glucagon to its receptor, thereby blocking the elevation of blood glucose levels. The antibodies or antigen binding fragments thereof may be useful for lowering blood glucose levels in a subject that suffers from a disease characterized by increased blood glucose levels, such as diabetes mellitus. The antibodies may also be used to treat a wide range of conditions and disorders in which blocking the interaction of glucagon with the glucagon receptor is desired, thereby having a beneficial effect. The antibodies may ultimately be used to prevent the long-term complications associated with elevated blood glucose levels in diabetic patients, or to ameliorate at least one symptom associated with elevated blood glucose levels in diabetic patients.

In another embodiment, the glucagon signaling pathway antagonist is a fully human monoclonal antibody (mAb) or antigen-binding fragments thereof that bind specifically to human glucagon (hGCG), to be used in combination with either a therapeutically effective amount of an inhibitor of an amino acid transporter, designated SLC38A5, or with a therapeutically effective amount of an inhibitor of mechanistic target of rapamycin (mTOR). The antibodies that bind specifically to glucagon inhibit or block its receptor binding activity, thereby blocking the elevation of blood glucose levels. The antibodies or antigen binding fragments thereof may be useful for lowering blood glucose levels in a subject that suffers from a disease characterized by increased blood glucose levels, such as diabetes mellitus. The antibodies may also be used to treat a wide range of conditions and disorders in which blocking the interaction of glucagon with the glucagon receptor is desired, thereby having a beneficial effect. The antibodies may ultimately be used to prevent the long-term complications associated with elevated blood glucose levels in diabetic patients, or to ameliorate at least one symptom associated with elevated blood glucose levels in diabetic patients.

The antibodies of the invention can be full-length (for example, an IgG1 or IgG4 antibody) or may comprise only an antigen-binding portion (for example, a Fab, F(ab′)₂ or scFv fragment), and may be modified to affect functionality, e.g., to eliminate residual effector functions (Reddy et al., 2000, J. Immunol. 164:1925-1933).

In a second aspect, the invention provides a method for lowering blood glucose levels, or for treating a condition or disease associated with, or characterized in part by high blood glucose levels, or at least one symptom or complication associated with the condition or disease, the method comprising administering a therapeutically effective amount of a glucagon signaling pathway antagonist, in combination with a therapeutically effective amount of an inhibitor of the amino acid transporter Solute Carrier Family 38 Member 5 (SLC38A5), or with a therapeutically effective amount of an inhibitor of mTOR, to a patient in need thereof, such that blood glucose levels are lowered or that the condition or disease is mediated, or at least one symptom or complication associated with the condition or disease is alleviated or reduced in severity.

In some embodiments, the glucagon signaling pathway antagonist is selected from a small molecule inhibitor, shRNA, siRNA, a peptide inhibitor, CRISPR technology (Clustered regularly interspaced short palindromic repeats; CRISPR technology can generate GCGR knock-down or deletion of regulatory sequences affecting GCGR activity), an antisense inhibitor, a DARPin, and a GCG inhibitor or a GCGR antagonist (such as a neutralizing monoclonal antibody).

In some embodiments, the GCGR antagonist can be an anti-GCGR antibody. The anti-GCGR antibody can inhibit or antagonize the GCGR. The anti-GCGR antibody can inhibit or block the GCGR signaling pathway. In some aspects, the GCG inhibitor can be an anti-GCG antibody. The anti-GCG antibody can inhibit binding of GCG to the GCGR.

In certain embodiments, the antibody or antigen-binding fragment specifically binds hGCGR, and comprises the heavy and light chain CDR domains contained within heavy and light chain sequence pairs selected from the group consisting of SEQ ID NO: 2/10, 18/26, 34/42, 50/58, 66/68, 70/78, 86/88, 90/98, 106/108, 110/118, 126/128, 130/138 and 146/148.

In certain embodiments, the antibody or antigen-binding fragment comprises the heavy and light chain CDR domains contained within the HCVR/LCVR amino acid sequence pair of SEQ ID NOs: 86/88.

In certain embodiments, the antibody or antigen-binding fragment comprises a HCVR/LCVR amino acid sequence pair of SEQ ID NOs: 86/88.

In one embodiment, the human antibody or antigen-binding fragment of a human antibody that binds hGCGR comprises a heavy chain variable region (HCVR) having an amino acid sequence selected from the group consisting of SEQ ID NO: 2, 18, 34, 50, 66, 70, 86, 90, 106, 110, 126, 130 and 146, or a substantially similar sequence thereof having at least 90%, at least 95%, at least 98% or at least 99% sequence identity.

In one embodiment, the human antibody or antigen-binding fragment of a human antibody that binds hGCGR comprises a light chain variable region (LCVR) having an amino acid sequence selected from the group consisting of SEQ ID NO: 10, 26, 42, 58, 68, 78, 88, 98, 108, 118, 128, 138 and 148, or a substantially similar sequence thereof having at least 90%, at least 95%, at least 98% or at least 99% sequence identity.

In certain embodiments, the human antibody or fragment thereof that binds hGCGR comprises a HCVR/LCVR amino acid sequence pair selected from the group consisting of SEQ ID NO: 2/10, 18/26, 34/42, 50/58, 66/68, 70/78, 86/88, 90/98, 106/108, 110/118, 126/128, 130/138, and 146/148. In certain embodiments, the HCVR/LCVR amino acid sequence pair is selected from the group consisting of SEQ ID NO: 34/42, 70/78, 86/88, 110/118 and 126/128.

In certain embodiments, the isolated human antibody or an antigen-binding fragment thereof that binds specifically to hGCGR comprises a HCVR comprising the three heavy chain CDRs (HCDR1, HCDR2 and HCDR3) contained within the HCVR sequence selected from the group consisting of SEQ ID NO: 2, 18, 34, 50, 66, 70, 86, 90, 106, 110, 126, 130 and 146; and/or a LCVR comprising the three light chain CDRs (LCDR1, LCDR2 and LCDR3) contained within the LCVR sequences selected from the group consisting of SEQ ID NO: 10, 26, 42, 58, 68, 78, 88, 98, 108, 118, 128, 138 and 148.

In certain embodiments, the methods provided herein contemplate the use of an isolated human antibody or antigen-binding fragment thereof that binds hGCGR comprising a HCDR3 domain having an amino acid sequence selected from the group consisting of SEQ ID NO: 8, 24, 40, 56, 76, 96, 116 and 136, or a substantially similar sequence thereof having at least 90%, at least 95%, at least 98% or at least 99% sequence identity; and/or a LCDR3 domain having an amino acid sequence selected from the group consisting of SEQ ID NO: 16, 32, 48, 64, 84, 104, 124 and 144, or a substantially similar sequence thereof having at least 90%, at least 95%, at least 98% or at least 99% sequence identity.

In one embodiment, the methods provided herein contemplate use of an antibody or fragment thereof that further comprises a HCDR1 domain having an amino acid sequence selected from the group consisting of SEQ ID NO: 4, 20, 36, 52, 72, 92, 112 and 132, or a substantially similar sequence thereof having at least 90%, at least 95%, at least 98% or at least 99% sequence identity; a HCDR2 domain having an amino acid sequence selected from the group consisting of SEQ ID NO: 6, 22, 38, 54, 74, 94, 114 and 134, or a substantially similar sequence thereof having at least 90%, at least 95%, at least 98% or at least 99% sequence identity; a LCDR1 domain having an amino acid sequence selected from the group consisting of SEQ ID NO: 12, 28, 44, 60, 80, 100, 120 and 140, or a substantially similar sequence thereof having at least 90%, at least 95%, at least 98% or at least 99% sequence identity; and a LCDR2 domain having an amino acid sequence selected from the group consisting of SEQ ID NO: 14, 30, 46, 62, 82, 102, 122 and 142, or a substantially similar sequence thereof having at least 90%, at least 95%, at least 98% or at least 99% sequence identity.

In one embodiment, the antibody or antigen-binding fragment of an antibody comprises:

(a) a HCDR3 domain having an amino acid sequence selected from the group consisting of SEQ ID NO: 8, 24, 40, 56, 76, 96, 116 and 136; and
(b) a LCDR3 domain having an amino acid sequence selected from the group consisting of SEQ ID NO: 16, 32, 48, 64, 84, 104, 124 and 144.

In a related embodiment, the antibody or antigen-binding fragment of the antibody further comprises:

(c) a HCDR1 domain having an amino acid sequence selected from the group consisting of SEQ ID NO: 4, 20, 36, 52, 72, 92, 112 and 132;
(d) a HCDR2 domain having an amino acid sequence selected from the group consisting of SEQ ID NO: 6, 22, 38, 54, 74, 94, 114 and 134;
(e) a LCDR1 domain having an amino acid sequence selected from the group consisting of SEQ ID NO: 12, 28, 44, 60, 80, 100, 120 and 140; and
(f) a LCDR2 domain having an amino acid sequence selected from the group consisting of SEQ ID NO: 14, 30, 46, 62, 82, 102, 122 and 142.

In one embodiment, the antibody or antigen-binding fragment thereof comprises a HCVR comprising a HCDR1 domain having an amino acid sequence selected from one of SEQ ID NO: 4, 20, 36, 52, 72, 92, 112 and 132; a HCDR2 domain having an amino acid sequence selected from one of SEQ ID NO: 6, 22, 38, 54, 74, 94, 114 and 134; a HCDR3 domain having an amino acid sequence selected from one of SEQ ID NOs: 8, 24, 40, 56, 76, 96, 116 and 136; and a LCVR comprising a LCDR1 domain having an amino acid sequence selected from one of SEQ ID NO: 12, 28, 44, 60, 80, 100, 120 and 140; a LCDR2 domain having an amino acid sequence selected from one of SEQ ID NO: 14, 30, 46, 62, 82, 102, 122 and 142; and a LCDR3 domain having an amino acid sequence selected from one of SEQ ID NO: 16, 32, 48, 64, 84, 104, 124 and 144.

In certain embodiments, the human antibody or antigen-binding fragment of a human antibody that binds to human GCGR comprises a HCDR3/LCDR3 amino acid sequence pair selected from the group consisting of SEQ ID NO: 8/16, 24/32, 40/48, 56/64, 76/84, 86/88, 96/104, 116/124 and 136/144. Non-limiting examples of anti-GCGR antibodies having these HCDR3/LCDR3 pairs are the antibodies designated H4H1345N, H4H1617N, H4H1765N, H4H1321B and H4H1321P, H4H1327B and H4H1327P, H4H1328B and H4H1328P, H4H1331B and H4H1331P, H4H1339B and H4H1339P, respectively.

In one embodiment, the isolated antibody or antigen-binding fragment thereof useful according to the methods provided herein, that specifically binds to GCG and neutralizes at least one activity associated with GCG, comprises: (a) three heavy chain complementarity determining regions (HCDR1, HCDR2 and HCDR3) contained within a heavy chain variable region (HCVR) amino acid sequence selected from the group consisting of SEQ ID NOs: 150, 166, 182, 198, 214, 230, 246, 262, 278 and 294; and (b) three light chain CDRs (LCDR1, LCDR2 and LCDR3) contained within a light chain variable region (LCVR) amino acid sequence selected from the group consisting of SEQ ID NOs: 158, 174, 190, 206, 222, 238, 254, 270, 286 and 302.

In some embodiments, the isolated antibody or antigen-binding fragment thereof that specifically binds to GCG and neutralizes at least one activity associated with GCG, comprises an HCVR having an amino acid sequence selected from the group consisting of SEQ ID NOs: 150, 166, 182, 198, 214, 230, 246, 262, 278 and 294 and a LCVR having an amino acid sequence selected from the group consisting of SEQ ID NOs: 158, 174, 190, 206, 222, 238, 254, 270, 286 and 302.

In some embodiments, the isolated antibody or antigen-binding fragment thereof that specifically binds to GCG and neutralizes at least one activity associated with GCG, comprises a HCVR/LCVR amino acid sequence pair selected from the group consisting of SEQ ID NOs: 150/158; 166/174; 182/190; 198/206; 214/222; 230/238; 246/254; 262/270; 278/286 and 294/302.

In some embodiments, the HCVR/LCVR amino acid sequence pair comprises SEQ ID NOs: 166/174.

In some embodiments, the HCVR/LCVR amino acid sequence pair comprises SEQ ID NOs: 182/190.

In one embodiment, the isolated antibody or antigen-binding fragment thereof useful according to the methods provided herein, comprises:

(a) a HCDR1 domain having an amino acid sequence selected from the group consisting of SEQ ID NOs: 152, 168, 184, 200, 216, 232, 248, 264, 280, and 296;
(b) a HCDR2 domain having an amino acid sequence selected from the group consisting of SEQ ID NOs: 154, 170, 186, 202, 218, 234, 250, 266, 282, and 298;
(c) a HCDR3 domain having an amino acid sequence selected from the group consisting of SEQ ID NOs: 156, 172, 188, 204, 220, 236, 252, 268, 284, and 300;
(d) a LCDR1 domain having an amino acid sequence selected from the group consisting of SEQ ID NOs: 160, 176, 192, 208, 224, 240, 256, 272, 288, and 304;
(e) a LCDR2 domain having an amino acid sequence selected from the group consisting of SEQ ID NOs: 162, 178, 194, 210, 226, 242, 258, 274, 290, and 306; and
(f) a LCDR3 domain having an amino acid sequence selected from the group consisting of SEQ ID NOs: 164, 180, 196, 212, 228, 244, 260, 276, 292, and 308.

In one embodiment, the isolated antibody or antigen-binding fragment thereof useful according to the methods provided herein, comprises:

(a) a HCDR1 domain comprising the amino acid sequence of SEQ ID NO: 168;
(b) a HCDR2 domain comprising the amino acid sequence of SEQ ID NO: 170;
(c) a HCDR3 domain comprising the amino acid sequence of SEQ ID NO: 172;
(d) a LCDR1 domain comprising the amino acid sequence of SEQ ID NO: 176;
(e) a LCDR2 domain comprising the amino acid sequence of SEQ ID NO: 178; and
(f) a LCDR3 domain comprising the amino acid sequence of SEQ ID NO: 180.

In one embodiment, the isolated antibody or antigen-binding fragment thereof useful according to the methods provided herein, comprises:

(a) a HCDR1 domain comprising the amino acid sequence of SEQ ID NO: 184;
(b) a HCDR2 domain comprising the amino acid sequence of SEQ ID NO: 186;
(c) a HCDR3 domain comprising the amino acid sequence of SEQ ID NO: 188;
(d) a LCDR1 domain comprising the amino acid sequence of SEQ ID NO: 192;
(e) a LCDR2 domain comprising the amino acid sequence of SEQ ID NO: 194; and
(f) a LCDR3 domain comprising the amino acid sequence of SEQ ID NO: 196.

Also useful according to the methods provided herein are antibodies or antigen-binding fragments thereof that specifically bind GCG, comprising a heavy chain CDR1 (HCDR1) comprising an amino acid sequence selected from any of the HCDR1 amino acid sequences provided herein or a substantially similar sequence thereof having at least 90%, at least 95%, at least 98% or at least 99% sequence identity.

Also useful according to the methods provided herein are antibodies or antigen-binding fragments thereof that specifically bind GCG, comprising a heavy chain CDR2 (HCDR2) comprising an amino acid sequence selected from any of the HCDR2 amino acid sequences provided herein or a substantially similar sequence thereof having at least 90%, at least 95%, at least 98% or at least 99% sequence identity.

Also useful according to the methods provided herein are antibodies or antigen-binding fragments thereof that specifically bind GCG, comprising a heavy chain CDR3 (HCDR3) comprising an amino acid sequence selected from any of the HCDR3 amino acid sequences provided herein or a substantially similar sequence thereof having at least 90%, at least 95%, at least 98% or at least 99% sequence identity.

Also useful according to the methods provided herein are antibodies or antigen-binding fragments thereof that specifically bind GCG, comprising a light chain CDR1 (LCDR1) comprising an amino acid sequence selected from any of the LCDR1 amino acid sequences provided herein or a substantially similar sequence thereof having at least 90%, at least 95%, at least 98% or at least 99% sequence identity.

Also useful according to the methods provided herein are antibodies or antigen-binding fragments thereof that specifically bind GCG, comprising a light chain CDR2 (LCDR2) comprising an amino acid sequence selected from any of the LCDR2 amino acid sequences provided herein or a substantially similar sequence thereof having at least 90%, at least 95%, at least 98% or at least 99% sequence identity.

Also useful according to the methods provided herein are antibodies or antigen-binding fragments thereof that specifically bind GCG, comprising a light chain CDR3 (LCDR3) comprising an amino acid sequence selected from any of the LCDR3 amino acid sequences listed herein or a substantially similar sequence thereof having at least 90%, at least 95%, at least 98% or at least 99% sequence identity.

Also useful according to the methods provided herein are antibodies or antigen-binding fragments thereof that specifically bind GCG, comprising an HCDR3 and an LCDR3 amino acid sequence pair (HCDR3/LCDR3) comprising any of the HCDR3 amino acid provided herein paired with any of the LCDR3 amino acid sequences provided herein. According to certain embodiments, the antibodies, or antigen-binding fragments thereof, comprise an HCDR3/LCDR3 amino acid sequence pair contained within any of the exemplary anti-GCG antibodies provided herein. In certain embodiments, the HCDR3/LCDR3 amino acid sequence pair comprises SEQ ID NOs: 172/180.

Also useful according to the methods provided herein are antibodies or antigen-binding fragments thereof that specifically bind GCG, comprising a set of six CDRs (i.e., HCDR1-HCDR2-HCDR3-LCDR1-LCDR2-LCDR3) contained within any of the exemplary anti-GCG antibodies provided herein. In certain embodiments, the HCDR1/HCDR2/HCDR3/LCDR1/LCDR2/LCDR3 amino acid sequence set comprises SEQ ID NOs: 168/170/172/176/178/180. In certain embodiments, the HCDR1/HCDR2/HCDR3/LCDR1/LCDR2/LCDR3 amino acid sequence set comprises SEQ ID NOs: 184/186/188/192/194/196.

In a related embodiment, the antibodies, or antigen-binding fragments thereof that specifically bind GCG, comprise a set of six CDRs (i.e., HCDR1/HCDR2/HCDR3/LCDR1/LCDR2/LCDR3) contained within an HCVR/LCVR amino acid sequence pair as defined by any of the exemplary anti-GCG antibodies provided herein. For example, the antibodies or antigen-binding fragments thereof that specifically bind GCG, comprise the HCDR1/HCDR2/HCDR3/LCDR1/LCDR2/LCDR3 amino acid sequences set contained within an HCVR/LCVR amino acid sequence pair selected from the group consisting of: 166/174; 182/190; 198/206; 214/222; 230/238; 246/254; 262/270; 278/286 and 294/302.

Non-limiting examples of antibodies that specifically bind GCG and comprise the CDR sequences provided above, include HIH059P, H4H10223P, H4H10231P, H4H10232P, H4H10236P, H4H10237P, H4H10238P, H4H10250P, H4H10256P, and H4H10270P.

Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, (1991) “Sequences of Proteins of Immunological Interest,” National Institutes of Health, Bethesda, Md.; Al-Lazikani et al., (1997) J. Mol. Biol. 273:927-948; and Martin et al., (1989) Proc. Natl. Acad. Sci. USA 86:9268-9272. Public databases are also available for identifying CDR sequences within an antibody.

In one embodiment, the condition or disease is selected from the group consisting of diabetes, impaired glucose tolerance, obesity, nephropathy, neuropathy, retinopathy, cataracts, stroke, atherosclerosis, impaired wound healing, diabetic ketoacidosis, hyperglycemia, hyperglycemic hyperosmolar syndrome, perioperative hyperglycemia, hyperglycemia in the intensive care unit patient, hyperinsulinemia, the metabolic syndrome, insulin resistance syndrome and impaired fasting glucose.

In one embodiment, the inhibitor of SLC38A5 is selected from the group consisting of a small organic molecule, a protein, a polypeptide, an antibody, an siRNA and an antisense molecule.

In one embodiment, the GCGR antagonist antibody is administered subcutaneously, intravenously or intramuscularly.

In one embodiment, the SLC38A5 inhibitor is administered orally, subcutaneously, intravenously or intramuscularly.

In one embodiment, the glucagon signaling pathway antagonist, such as a GCGR antagonist antibody or anti-glucagon antibody, and the SLC38A5 inhibitor are administered concurrently or sequentially.

In one embodiment, the GCGR antagonist antibody or the anti-glucagon antibody and the SLC38A5 inhibitor are administered at therapeutically effective concentrations in separate pharmaceutical compositions or are co-formulated in one pharmaceutical composition.

In one embodiment, the method provides for administration of one or more additional therapeutic agents. The one or more therapeutic agents may be selected from the group consisting of insulin, a biguanide (metformin), a sulfonylurea (such as glyburide, glipizide), a PPAR gamma agonist (pioglitazone, rosiglitazone), an alpha glucosidase inhibitor (acarbose, voglibose), EXENATIDE® (glucagon-like peptide 1), SYMLIN® (pramlintide), a glucagon antagonist, and a second GCGR antagonist. The one or more therapeutic agents may be a 3-hydroxy-3-methyl-glutaryl-CoA reductase (HMG-CoA reductase) inhibitor. The HMG-CoA reductase inhibitor is a statin selected from the group consisting of atorvastatin, fluvastatin, lovastatin, pitavastatin, pravastatin, rosuvastatin and simvastatin.

In one embodiment, the methods of the invention provide for lowering blood glucose levels, or for treating a condition or disease associated with, or characterized in part, by high blood glucose levels and results in a reduction in blood glucose levels without demonstrating an increase in alpha cell hyperplasia.

The glucagon signaling pathway antagonists as described herein may also be useful for treating patients with inoperable glucagonoma (pancreatic endocrine tumor with or without necrolytic migratory erythema and hyperglycemia).

Other embodiments will become apparent from a review of the ensuing detailed description.

### BRIEF DESCRIPTION OF THE FIGURES

FIG. 1. Expression of Genes Involved in Amino Acid Transporter, Catabolism, Gluconeogenesis and Urea Cycle in Liver From GCGR Antibody Treated Mice

Differentially regulated (A) amino acid transporter, (B) amino acid catabolism (C) urea cycle and (D) gluconeogenesis genes in livers of mice treated with GCGR antibody (15 mg/kg) for 21 days. The gene expression was compared to mice treated with control antibody. Genes with expression >1 RPKM were included (in addition, amino acid metabolism genes were further selected by fold change >1.5) (n=4).

FIG. 2. Expression of Slc38a5 in Intact Islets and α-Cells from GCGR Antibody Treated Mice

(A) Differentially regulated genes in intact islets of mice treated with GCGR antibody (10 mg/kg) for 21 days. The gene expression was compared to mice treated with control antibody. Inclusion criteria were average baseline expression >10 normalized counts, fold change >1.5 and p<0.01 (n=5).

(B) Changes in the expression of amino acid transporters in islets of mice treated with GCGR or control antibody for 21 days. Transporters with expression >1 RPKM were included.

(C) Immunofluorescence staining of pancreas sections for glucagon and Slc38a5 in mice treated with GCGR or control antibody (3 mg/kg) for 21 days.

(D) Percentage of Slc38a5 positive α-cells. Data are mean±SEM. GCGR antibody (95 islets/pancreas from n=15 pancreata), control antibody (30 islets/pancreas from n=12 pancreata). ****p<0.0001.

FIG. 3. α-Cells Expressing Slc38a5 Show High Rate of Proliferation

(A) Immunofluorescence staining for glucagon, Slc38a5 and Ki67 (white) in pancreas sections from mice treated for 11 days with GCGR or control antibody.

(B) Representative proliferating α-cells staining positive for SLC38A5 and Ki67.

(C) Percentage of Ki67 positive cells among glucagon negative, α-cells with no detectable Slc38a5 expression, and α-cells with detectable Slc38a5 expression. Data are mean±SEM, 10 islets from n=4 pancreata/group. **p<0.01, ***p<0.001.

(D) Proliferation of αTC1-6 glucagonoma cells with reduced (KD) or normal (WT) expression of Slc38a5 for 5 days. Data are mean±SEM, n=4. **p<0.01, ****p<0.0001.

FIG. 4. mTORC1 Inhibition by Rapamycin Prevents GCGR Inhibition-Induced α-Cell Hyperplasia in Mice

(A-C, F) Blood glucose, plasma amino acids, glucagon and insulin levels before and 21 days following weekly injections of GCGR antibody or control (3 mg/kg) in combination with PBS or rapamycin (10 mg/kg, daily). Data are mean±SEM, n=5-10. ****p<0.0001.

(D) Glucagon, Slc38a5 and DAPI nuclei immunofluorescence staining of representative pancreas sections from mice treated with GCGR or control antibody in combination with PBS or rapamycin.

(E, G) α-cell mass and size in mice treated as described in (A). Data are mean±SEM, n=8-10. ****p<0.0001.

(H) Slc38a5 mRNA expression levels in intact islets from mice treated as described in (A). Data are mean±SEM, n=3. ****p<0.0001.

(I) Percentage of Slc38a5 positive α-cells in islets from mice treated as described in (A). Data are mean±SEM, 30-95 islets from n=6-15 pancreata/group. ****p<0.0001.

FIG. 5. α-Cell Mass is Reduced in Slc38a5^{-/-} Mice Treated With GCGR Antibody

(A-D) Blood glucose, plasma amino acids, glucagon and insulin levels of wildtype (WT) and Slc38a5 KO mice before and 20 days following weekly injections of control or GCGR antibody (10 mg/kg). Data are mean±SEM, n=5-8. **p<0.01, ****p<0.0001.

(E) Glucagon), Slc38a5 and DAPI nuclei immunofluorescence staining of pancreas sections from WT and Slc38a5^{-/-} mice treated with GCGR or control antibody.

(F-I) α-cell mass, α-cell size, α-cell mass and islet number per pancreas section in WT and Slc38a5^{-/-} mice treated with GCGR or control antibody. Data are mean±SEM, n=6-8. **p<0.01, ***p<0.001, ****p<0.0001.

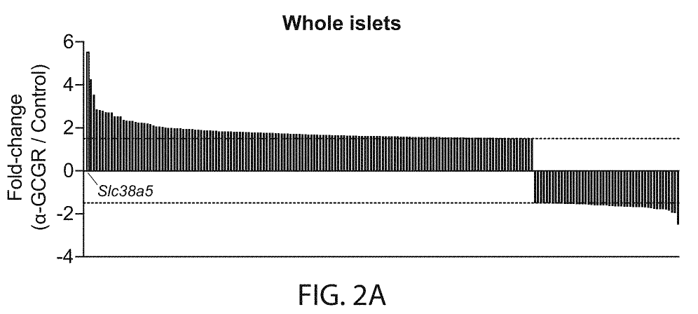

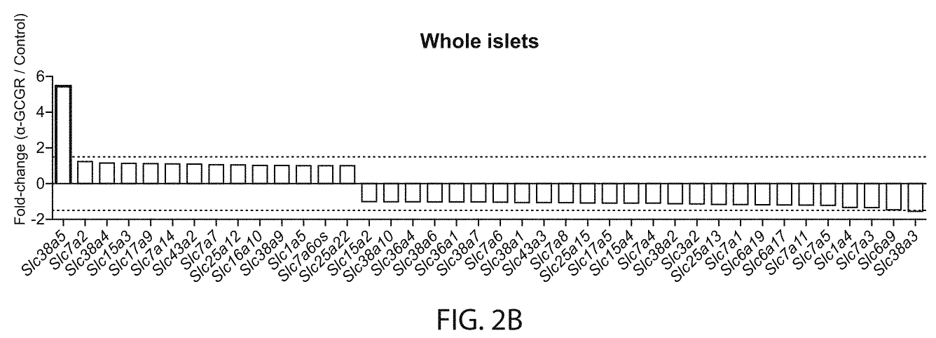

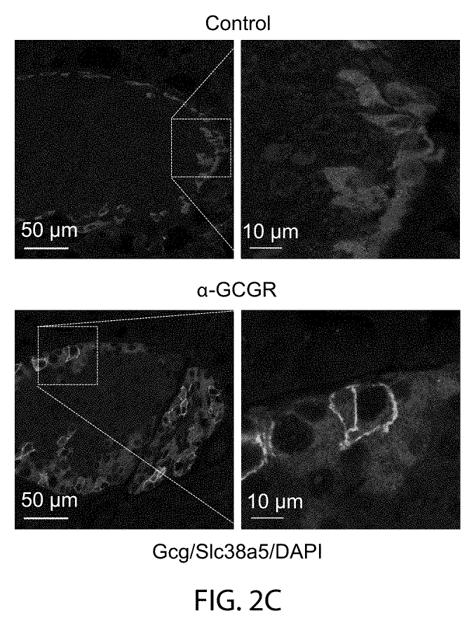

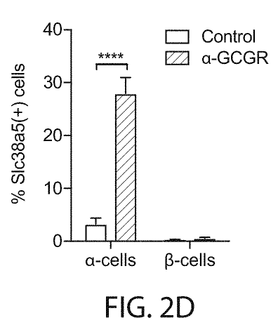

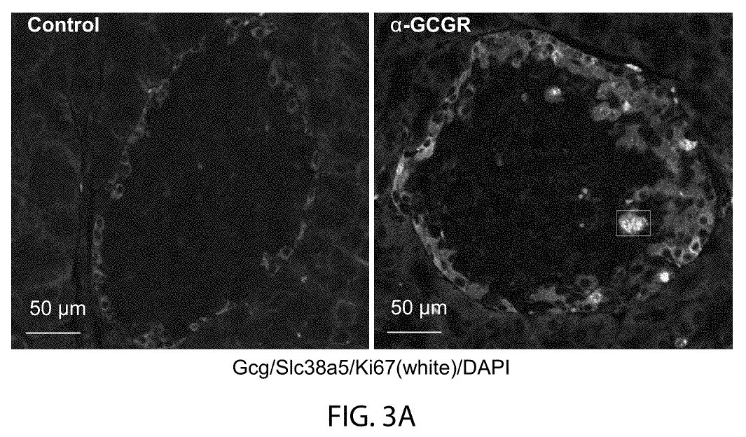

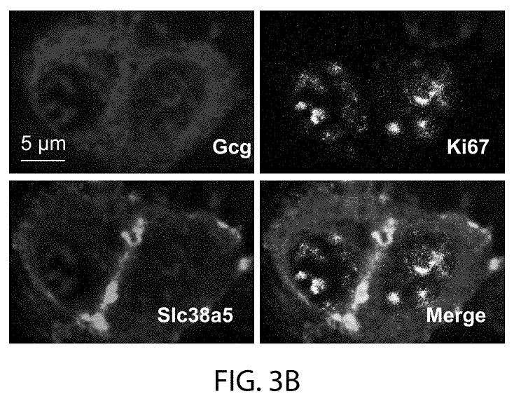

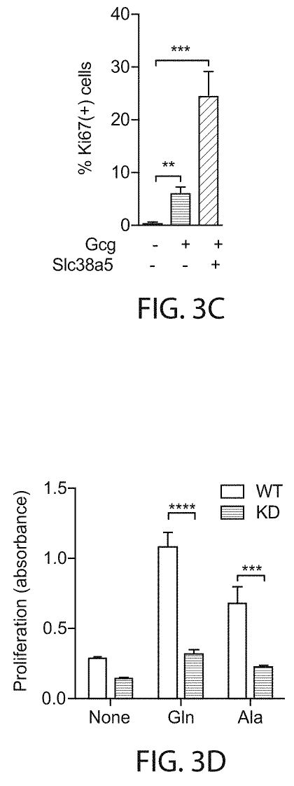

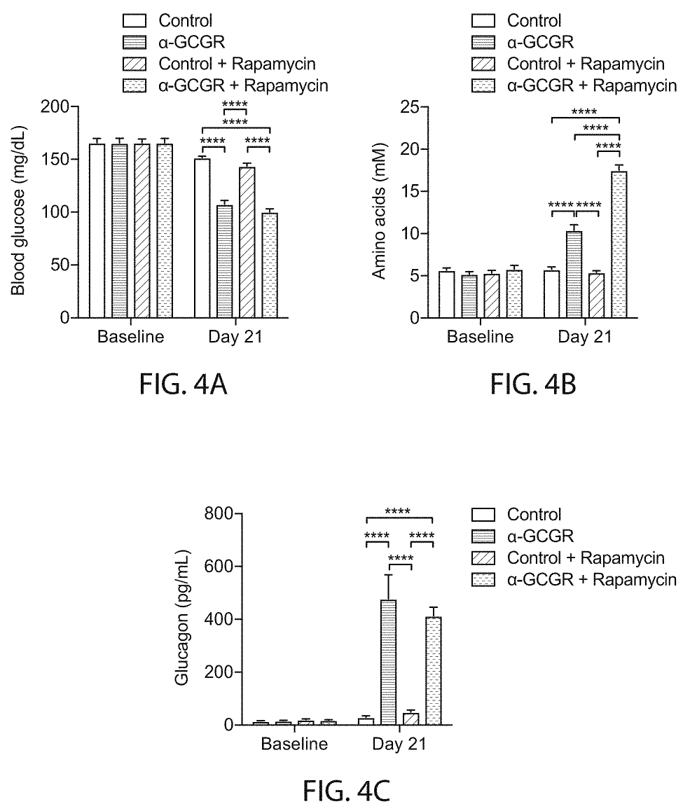

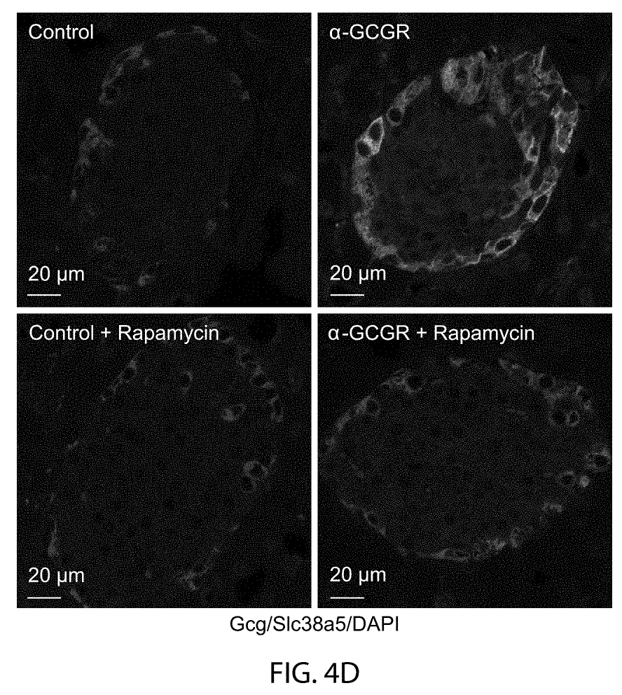

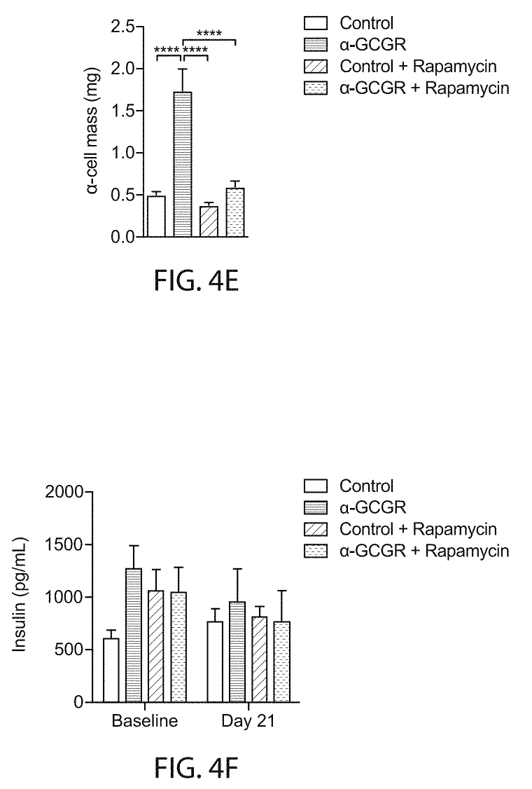

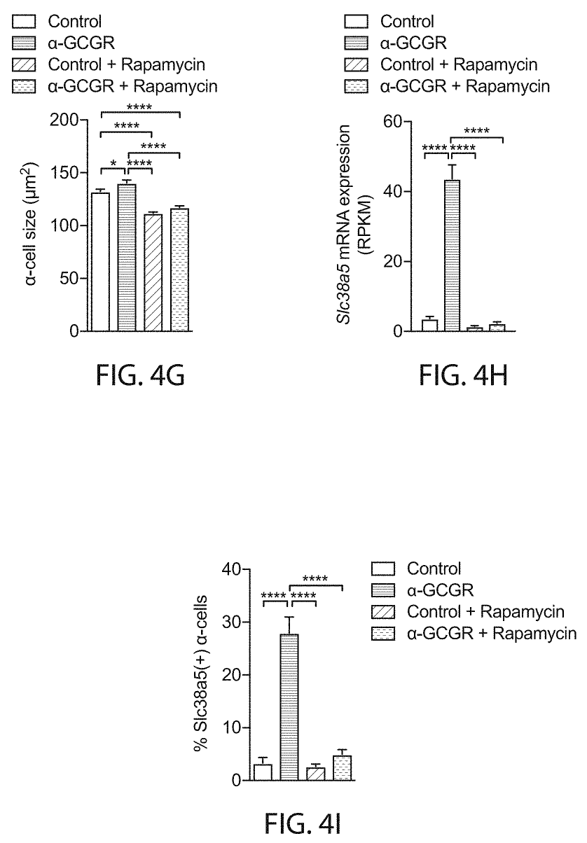

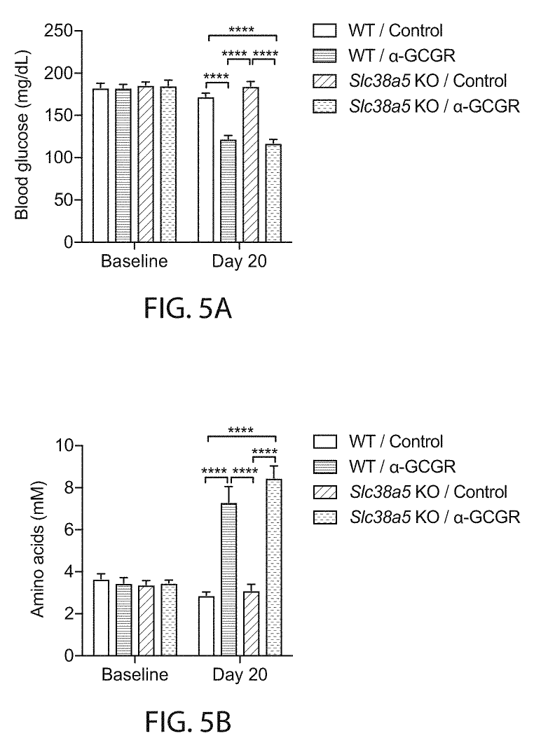

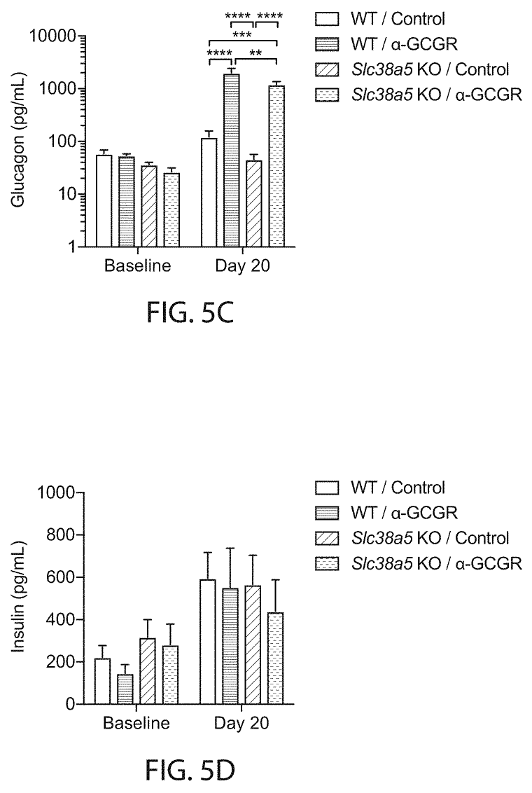

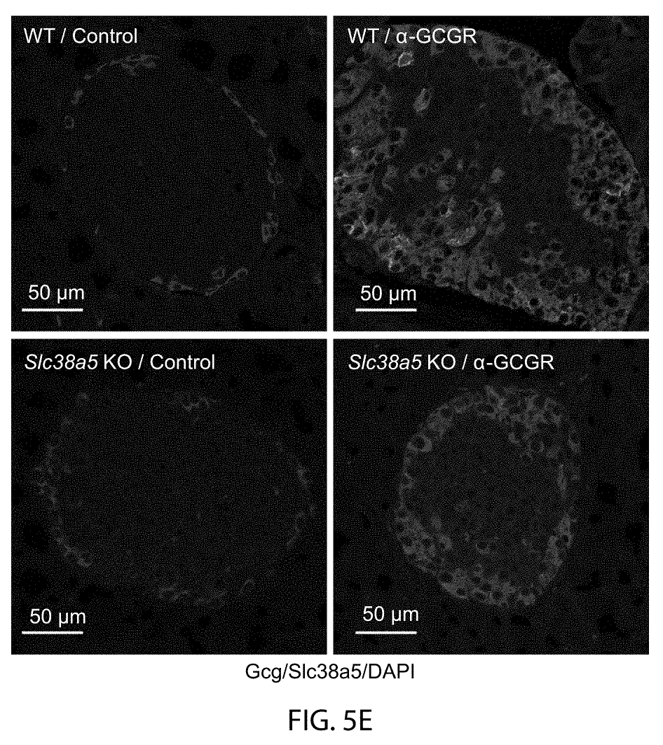

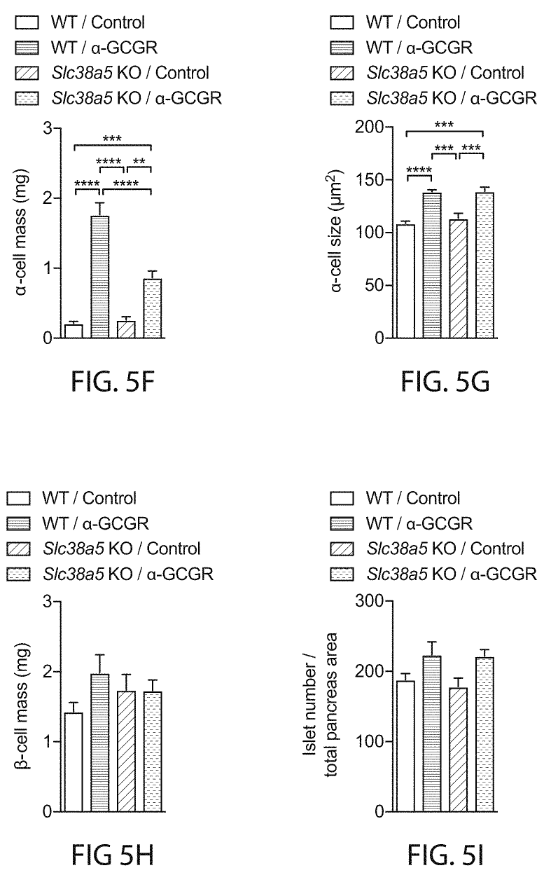

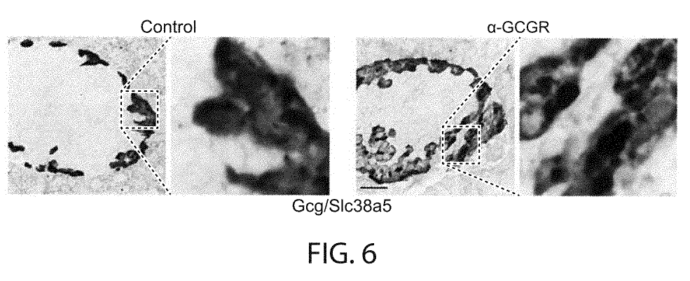

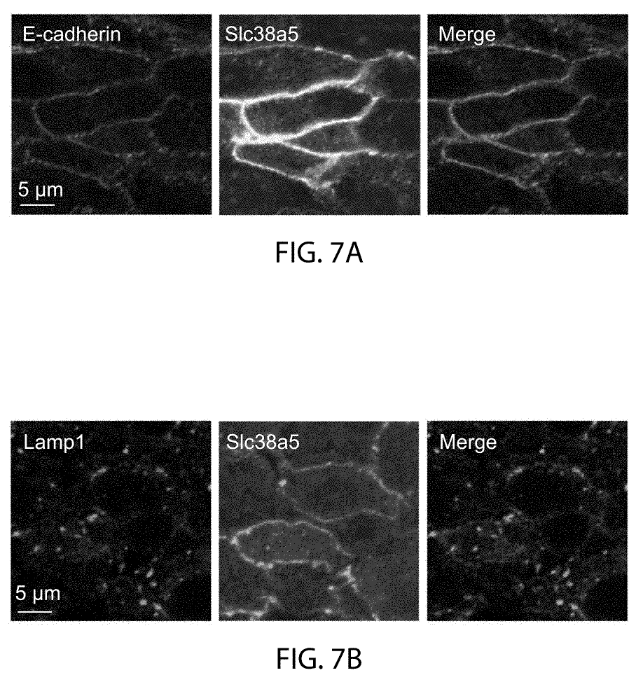

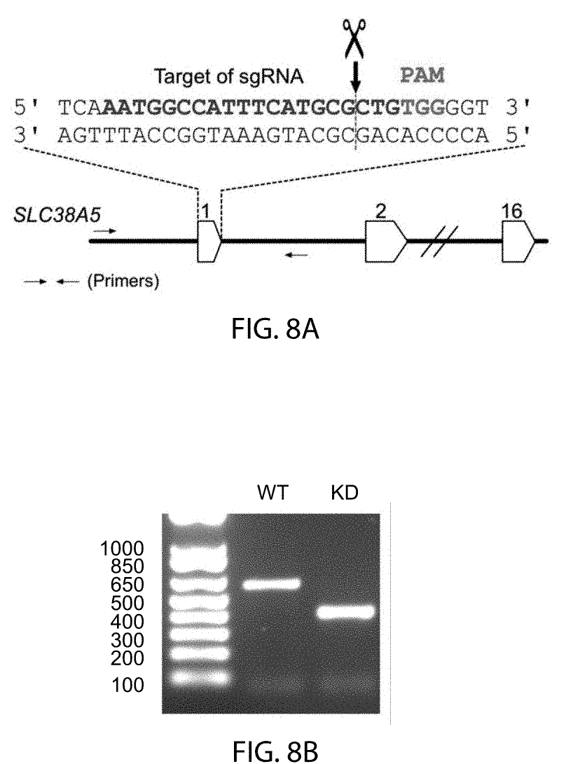

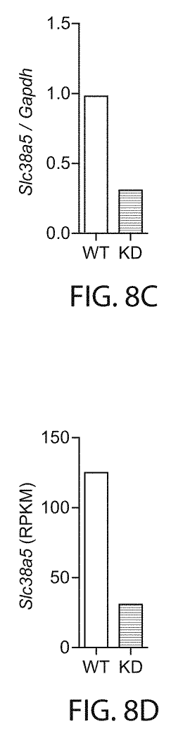

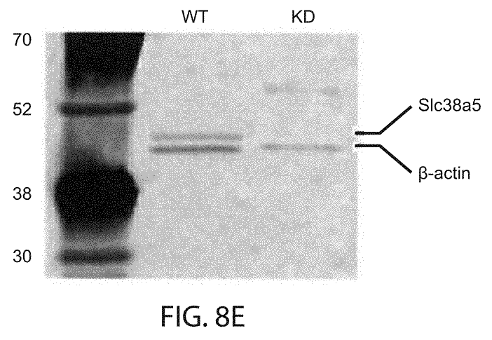

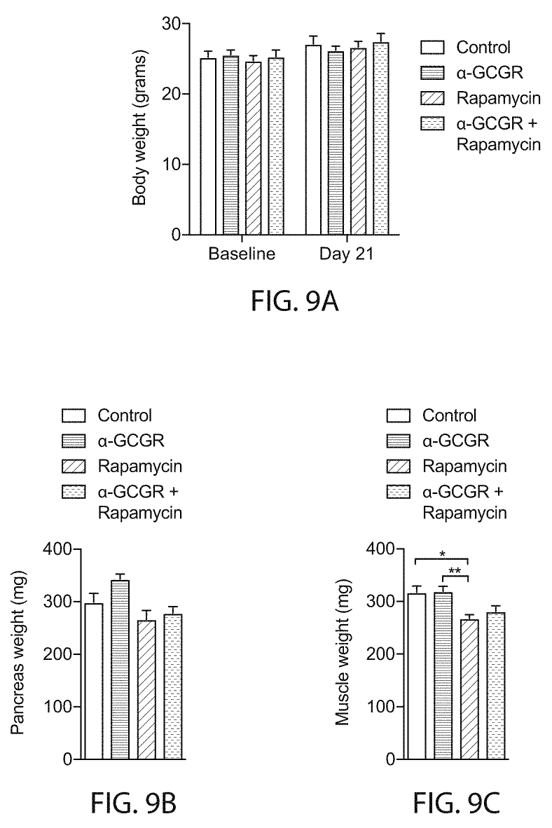

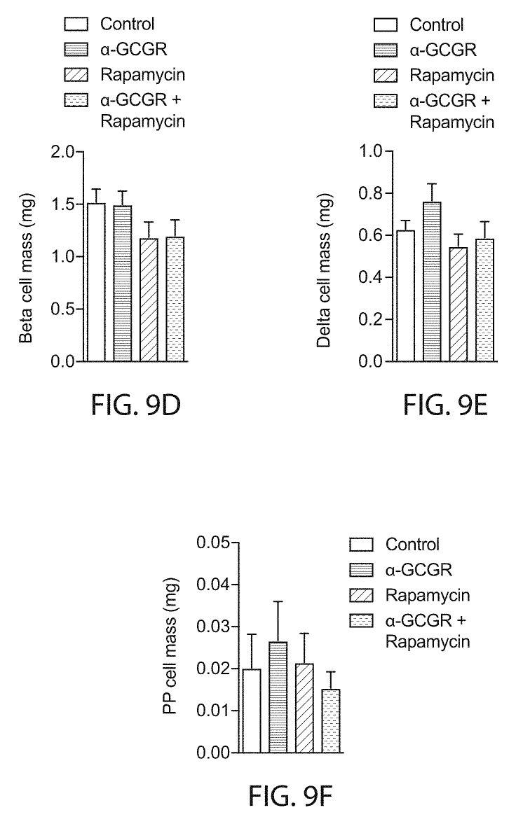

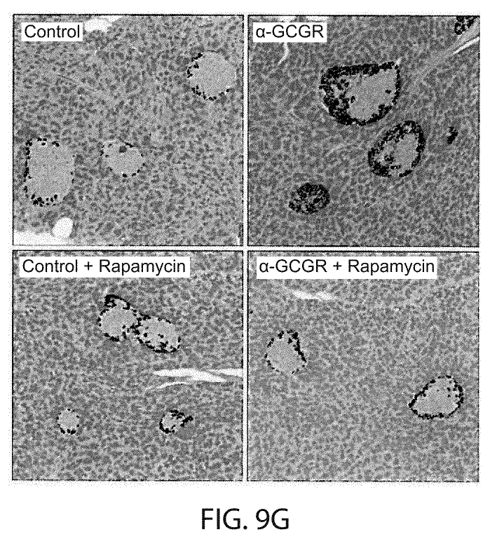

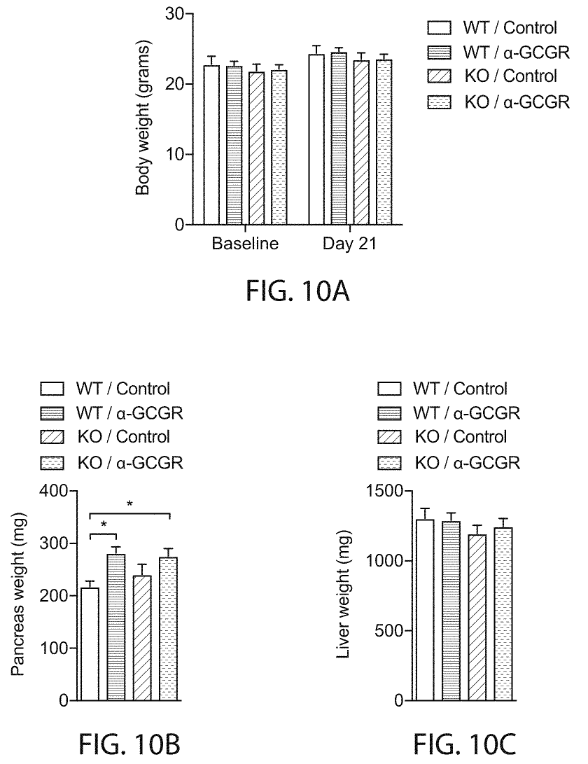

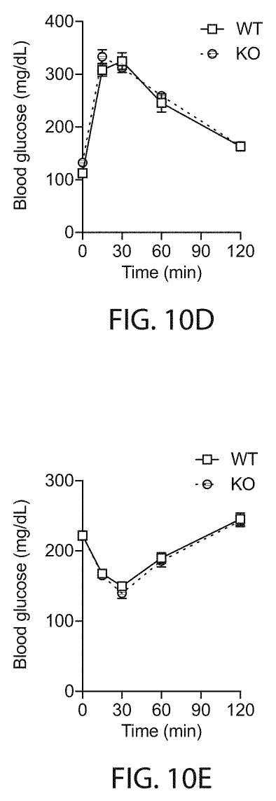

FIG. 6, related to FIG. 2. GCGR Antibody-Induced Slc38a5 Expression in α-Cells

Slc38a5 and glucagon mRNA transcripts were stained on pancreas sections from mice treated with GCGR or control antibody using RNAscope.

FIG. 7, related to FIG. 2. Subcellular Localization of Slc38a5

(A) Double immunofluorescence stating of pancreas sections from mice treated with GCGR antibody for Slc38a5 and E-cadherin (plasma membrane marker).

(B) Slc38a5 and Lamp1 (lysosomal membrane marker) to determine subcellular localization of Slc38a5.

FIG. 8, related to FIG. 3. Generation of Slc38a5 Knock-Down (KD) αTC16 Glucagonoma Cell Line Using CRISPR-Cas9

(A) Schematic of single-guide RNA targeting the exon1 of Slc38a5 gene to induce deletion mutation.

(B) Verification of deletion mutation in Slc38a5 by PCR amplification.

(C-D) TaqMan and RNAseq analyse to confirm the reduction of Slc38a5 in KD αTC16 clone.

(E) Western blot for Slc38a5 in WT and KD.

FIG. 9, related to FIG. 4. GCGR Inhibition-Induced α-Cell Hyperplasia is mTORC1-Dependent

(A) Body weights before and 3 weeks following weekly injections of either GCGR or control antibody (3 mg/kg) in combination with PBS or rapamycin (10 mg/kg, daily).

(B-F) Pancreas weight, muscle weight, α-, β- and PP cell mass from mice treated with GCGR or control antibody for 21 days. Data are mean±SEM, n=5-10.

(G) Glucagon immunohistochemistry of representative pancreas sections.

FIG. 10, related to FIG. 5. Slc38a5 Deficiency Does Not Affect Body or Organ Weights and Glycemic Control

(A) Body weights of WT and Slc38a5^{-/-} mice before and 3 weeks following weekly injections of either GCGR or control antibody (10 mg/kg). Data are mean±SEM, n=8.

(B-C) Pancreas and liver weights. Data are mean±SEM, n=7-8. *p<0.05.

(D-E) Oral glucose tolerance test and insulin tolerance test in chow fed WT and Slc38a5^{-/-} mice. Glucose was given orally (2 g/kg body weight) and insulin (0.75 U/kg of body weight) was administered by intraperitoneal (ip) injection. Data are mean±SEM, n=10-11.

### DETAILED DESCRIPTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, preferred methods and materials are now described. All publications mentioned herein are incorporated herein by reference in their entirety.

### Definitions

The “glucagon receptor”, also referred to herein as “GCGR”, belongs to the G protein-coupled receptor class 2 family and consists of a long amino terminal extracellular domain, seven transmembrane segments, and an intracellular C-terminal domain (Jelinek et al., Science 259: 1614-1616 (1993), Segre et al., Trends Endocrinol. Metab 4:309-314 (1993)). Glucagon receptors are notably expressed on the surface of hepatocytes where they bind to glucagon and transduce the signal provided thereby into the cell. Accordingly, the term “glucagon receptor” also refers to one or more receptors that interact specifically with glucagon to result in a biological signal. DNA sequences encoding glucagon receptors of rat and human origin have been isolated and disclosed in the art (EP0658200B1). The murine and cynomolgus monkey homologues have also been isolated and sequenced (Burcelin, et al., Gene 164 (1995) 305-310); McNally et al., Peptides 25 (2004) 1171-1178). As used herein, “glucagon receptor” and “GCGR” are used interchangeably. The expression “GCGR”, “hGCGR” or fragments thereof, as used herein, refers to the human GCGR protein or fragment thereof, unless specified as being from a non-human species, e.g. “mouse GCGR”, “rat GCGR”, or “monkey GCGR”. There are a variety of sequences related to the GCGR gene having the following Genbank Accession Numbers: NP_000151.1 (human), NP_742089.1 (rat), XP_001111894.1 (rhesus monkey), and NP_032127.2 (mouse). The nucleic acid sequences, the polypeptides encoded by them, and other nucleic acid and polypeptide sequences are herein incorporated by reference in their entireties as well as for individual subsequences contained therein.

The phrase “GCGR antagonist” refers to an inhibitor, antagonist, or inverse agonist of the GCGR signaling pathway. A “GCG inhibitor” may prevent the binding of glucagon to the receptor. A GCGR inhibitor may also prevent the binding of glucagon to the receptor. However, both effectively block or attenuate activation of the receptor, or may interfere with the signaling cascade downstream of the GCGR activation, and are collectively referred to as “glucagon signaling pathway antagonists”.

The terms “inhibitor” or “antagonist” include a substance that retards or prevents a chemical or physiological reaction or response, for example, a glucagon signaling pathway antagonist.

A GCGR antagonist is able to bind to the glucagon receptor and thereby antagonize the activity of GCG mediated by the GCGR. Inhibiting the activity of GCG by antagonizing the binding and activity of GCG at the GCGR reduces the rate of gluconeogenesis and glycogenolysis, and the concentration of glucose in plasma. Methods by which to determine the binding of a supposed antagonist with the glucagon receptor are known in the art and means by which to determine the interference with glucagon activity at the glucagon receptor are publicly available; see, e.g., S. E. de Laszlo et al., (1999) Bioorg. Med. Chem. Lett. 9:641-646.

Contemplated as useful herein are GCGR antagonists or GCG inhibitors having as a functional component thereof a small molecule compound, or in other words a low molecular weight organic compound. A small molecule is typically less than 800 Daltons. Additionally, CRISPR technology can be used to knock-down GCG or GCGR expression. As such, in some embodiments, a glucagon signaling pathway antagonist can be selected from a small molecule inhibitor, shRNA, siRNA, peptide inhibitor, CRISPR technology (Clustered regularly interspaced short palindromic repeats; CRISPR technology can generate GCGR knock-down or deletion of regulatory sequences affecting GCGR activity), an antisense inhibitor, DARPin, Spiegelmers, aptamers, engineered Fn type-III domains, GCG or GCGR neutralizing monoclonal antibodies, and their derivatives.

An example of a glucagon signaling pathway antagonist includes, but is not limited to, an antibody (human or humanized), or an antigen binding portion thereof, to GCG or GCGR, that blocks binding or inhibits the activity of the GCGR signaling pathway. Exemplary GCGR antagonists that may be used in the methods described herein include isolated human monoclonal antibody or antigen-binding fragment thereof comprising: (a) a HCVR having an amino acid sequence selected from the group consisting of SEQ ID NO: 2, 18, 34, 50, 66, 70, 86, 90, 106, 110, 126, 130 and 146; and/or (b) a LCVR having an amino acid sequence selected from the group consisting of SEQ ID NO: 10, 26, 42, 58, 68, 78, 88, 98, 108, 118, 128, 138 and 148. Exemplary GCG inhibitors that may be used in the methods described herein include isolated human monoclonal antibody or antigen-binding fragment thereof comprising: (a) a HCVR having an amino acid sequence selected from the group consisting of SEQ ID NO: 150, 166, 182, 198, 214, 230, 246, 262, 278, and 294; and/or (b) a LCVR having an amino acid sequence selected from the group consisting of SEQ ID NO: 158, 174, 190, 206, 222, 238, 254, 270, 286, and 302.

The term “antibody”, as used herein, is intended to refer to immunoglobulin molecules comprised of four polypeptide chains, two heavy (H) chains and two light (L) chains inter-connected by disulfide bonds (i.e., “full antibody molecules”), as well as multimers thereof (e.g. IgM) or antigen-binding fragments thereof. Each heavy chain is comprised of a heavy chain variable region (“HCVR” or “V_{H}”) and a heavy chain constant region (comprised of domains C_{H}1, C_{H}2 and C_{H}3). Each light chain is comprised of a light chain variable region (“LCVR or “V_{L}”) and a light chain constant region (C_{L}). The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In certain embodiments of the invention, the FRs of the anti-GCGR antibody (or antigen binding fragment thereof) may be identical to the human germline sequences, or may be naturally or artificially modified. An amino acid consensus sequence may be defined based on a side-by-side analysis of two or more CDRs.

Substitution of one or more CDR residues or omission of one or more CDRs is also possible. Antibodies have been described in the scientific literature in which one or two CDRs can be dispensed with for binding. Padlan et al. (1995 FASEB J. 9:133-139) analyzed the contact regions between antibodies and their antigens, based on published crystal structures, and concluded that only about one fifth to one third of CDR residues actually contact the antigen. Padlan also found many antibodies in which one or two CDRs had no amino acids in contact with an antigen (see also, Vajdos et al. 2002 J Mol Biol 320:415-428).

CDR residues not contacting antigen can be identified based on previous studies (for example residues H60-H65 in CDRH2 are often not required), from regions of Kabat CDRs lying outside Chothia CDRs, by molecular modeling and/or empirically. If a CDR or residue(s) thereof is omitted, it is usually substituted with an amino acid occupying the corresponding position in another human antibody sequence or a consensus of such sequences. Positions for substitution within CDRs and amino acids to substitute can also be selected empirically. Empirical substitutions can be conservative or non-conservative substitutions.

The fully-human anti-GCGR antibodies and anti-GCG antibodies disclosed herein may comprise one or more amino acid substitutions, insertions and/or deletions in the framework and/or CDR regions of the heavy and light chain variable domains as compared to the corresponding germline sequences. Such mutations can be readily ascertained by comparing the amino acid sequences disclosed herein to germline sequences available from, for example, public antibody sequence databases. The present invention includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present invention may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present invention.

Also provided herein are anti-hGCGR antibodies and anti-hGCG antibodies comprising variants of any of the HCVR, LCVR, and/or CDR amino acid sequences disclosed herein having one or more conservative substitutions. For example, the anti-hGCGR antibodies and anti-hGCG antibodies are contemplated as having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

The term “human antibody”, as used herein, is intended to include antibodies having variable and constant regions derived from human germline immunoglobulin sequences. The human mAbs of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include mAbs in which CDR sequences derived from the germline of another mammalian species (e.g., mouse), have been grafted onto human FR sequences. The anti-human GCGR antibodies may be designated as “anti-hGCGR” or “anti-GCGR”. The anti-human GCG antibodies may be designated herein as “anti-hGCG” or “anti-GCG”.

The term “specifically binds”, or the like, means that an antibody or antigen-binding fragment thereof forms a complex with an antigen that is relatively stable under physiologic conditions. Specific binding can be characterized by an equilibrium dissociation constant of at least about 1×10⁻⁶ M or less (e.g., a smaller K_{D} denotes a tighter binding). Methods for determining whether two molecules specifically bind are well known in the art and include, for example, equilibrium dialysis, surface plasmon resonance, and the like. An isolated antibody that specifically binds hGCGR may, however, exhibit cross-reactivity to other antigens such as GCGR molecules from other species. Moreover, multi-specific antibodies that bind to hGCGR and one or more additional antigens or a bi-specific that binds to two different regions of hGCGR are nonetheless considered antibodies that “specifically bind” hGCGR, as used herein. Likewise, an isolated antibody that specifically binds hGCG may exhibit cross-reactivity to other antigens such as GCG molecules from other species. Additionally, multi-specific antibodies that bind to hGCG and one or more additional antigens or a bi-specific that binds to two different regions of hGCG are nonetheless considered antibodies that “specifically bind” hGCG, as used herein.

The term “high affinity” antibody refers to those mAbs having a binding affinity to hGCGR, expressed as K_{D}, of at least 10⁻⁹ M; preferably 10⁻¹⁰ M; more preferably 10⁻¹¹ M, even more preferably 10⁻¹² M, as measured by surface plasmon resonance, e.g., BIACORE™ or solution-affinity ELISA.

By the term “slow off rate”, “Koff” or “kd” is meant an antibody that dissociates from hGCGR with a rate constant of 1×10⁻³ s⁻¹ or less, preferably 1×10⁻⁴ s⁻¹ or less, as determined by surface plasmon resonance, e.g., BIACORE™.

The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. The terms “antigen-binding portion” of an antibody, or “antibody fragment”, as used herein, refers to one or more fragments of an antibody that retain the ability to specifically bind to hGCGR or hGCG.

The specific embodiments, antibody or antibody fragments of the invention may be conjugated to a therapeutic moiety (“immunoconjugate”), such as a second GCGR antagonist, or to biguanide (metformin), a sulfonylurea (such as glyburide, glipizide), a PPAR gamma agonist (such as pioglitazone, or rosiglitazone), an alpha glucosidase inhibitor (such as acarbose, or voglibose), EXENATIDE® (glucagon-like peptide 1), SYMLIN® (pramlintide), a chemotherapeutic agent, a radioisotope, or any other therapeutic moiety useful for treating a disease or condition caused in part by unwanted glucagon activity.

An “isolated antibody”, as used herein, is intended to refer to an antibody that is substantially free of other antibodies (Abs) having different antigenic specificities (e.g., an isolated antibody that specifically binds hGCGR, or a fragment thereof, is substantially free of Abs that specifically bind antigens other than hGCGR).

A “blocking antibody” or a “neutralizing antibody”, as used herein (or an “antibody that neutralizes GCGR activity”), is intended to refer to an antibody whose binding to hGCGR or hGCG results in inhibition of at least one biological activity of GCGR. For example, an antibody of the invention may aid in preventing the increase in blood glucose levels associated with elevation of glucagon levels. Alternatively, an antibody of the invention may demonstrate the ability to block cAMP production in response to glucagon. This inhibition of the biological activity of GCGR can be assessed by measuring one or more indicators of GCGR biological activity by one or more of several standard in vitro or in vivo assays known in the art (see examples below).

The term “surface plasmon resonance”, as used herein, refers to an optical phenomenon that allows for the analysis of real-time biomolecular interactions by detection of alterations in protein concentrations within a biosensor matrix, for example using the BIACORE™ system (Pharmacia Biosensor AB, Uppsala, Sweden and Piscataway, N.J.).

The term “K_{D}”, as used herein, is intended to refer to the equilibrium dissociation constant of a particular antibody-antigen interaction.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 90% sequence identity, even more preferably at least 95%, 98% or 99% sequence identity. Preferably, residue positions, which are not identical, differ by conservative amino acid substitutions.

A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24: 307-331, which is herein incorporated by reference. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443 45, herein incorporated by reference. A “moderately conservative” replacement is any change having a nonnegative value in the PAM250 log-likelihood matrix.

Sequence similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215: 403 410 and (1997) Nucleic Acids Res. 25:3389 402, each of which is herein incorporated by reference.

In specific embodiments, the antibody or antibody fragment for use in the method of the invention may be mono-specific, bi-specific, or multi-specific. Multi-specific antibodies may be specific for different epitopes of one target polypeptide or may contain antigen-binding domains specific for epitopes of more than one target polypeptide. An exemplary bi-specific antibody format that can be used in the context of the present invention involves the use of a first immunoglobulin (Ig) C_{H}3 domain and a second Ig C_{H}3 domain, wherein the first and second Ig C_{H}3 domains differ from one another by at least one amino acid, and wherein at least one amino acid difference reduces binding of the bi-specific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise an Y96F modification (by IMGT; Y436F by EU). Further modifications that may be found within the second C_{H}3 include: D16E, L18M, N44S, K52N, V57M, and V82I (by IMGT; D356E, L358M, N384S, K392N, V397M, and V422I by EU) in the case of IgG1 mAbs; N44S, K52N, and V82I (IMGT; N384S, K392N, and V422I by EU) in the case of IgG2 mAbs; and Q15R, N44S, K52N, V57M, R69K, E79Q, and V82I (by IMGT; Q355R, N384S, K392N, V397M, R409K, E419Q, and V422I by EU) in the case of IgG4 mAbs. Variations on the bi-specific antibody format described above are contemplated within the scope of the present invention.

By the phrase “therapeutically effective amount” is meant an amount that produces the desired effect for which it is administered. The exact amount will depend on the purpose of the treatment, and will be ascertainable by one skilled in the art using known techniques (see, for example, Lloyd (1999) The Art, Science and Technology of Pharmaceutical Compounding). “Normal glucose levels” refers to mean plasma glucose values in humans of less than about 80 mg/dL for fasting levels, and about less than 110-120 mg/dL for post prandial levels. Plasma glucose may be determined in accordance with Etgen et al., (Metabolism 2000; 49(5): 684-688) or calculated from a conversion of whole blood glucose concentration in accordance with D'Orazio et al., (Clin. Chem. Lab. Med. 2006; 44(12): 1486-1490). In certain embodiments of the invention, the GCGR signaling antagonists such as anti-GCGR antibodies or anti-GCG antibodies may be useful to lower blood glucose levels to within the normal range. In one embodiment, the GCGR signaling antagonists may be useful to lower blood glucose levels to within the normal range and when combined for use with an inhibitor of the amino acid transporter SLC38A5, or when combined with an mTor inhibitor may do so without causing alpha cell hyperplasia.

The term “treating” (or “treat” or “treatment”) refers to processes involving a slowing, interrupting, inhibiting, arresting, controlling, stopping, reducing, ameliorating, or reversing the progression, duration, or severity of an existing symptom, disorder, condition, or disease, but does not necessarily involve a total elimination of all disease-related symptoms, conditions, or disorders through use of a GCG inhibitor or GCGR antagonist as described herein.

### GCG/GCGR Signaling Antagonists

Provided herein are GCG inhibitors and GCGR antagonists for the treatment of conditions or diseases characterized by in part by high glucose levels. In some embodiments, the antagonist is an inhibitor of glucagon. In some embodiments, the antagonist is an inhibitor of GCGR. In some embodiments, the GCGR antagonist is MK-0893, PF-06291874, LGD-6972, or LY2409021.

In some embodiments, the antagonist comprises an antibody capable of binding GCG or GCGR, or a fragment thereof. In some embodiments, the signaling pathway is inhibited by the interruption of GCG or GCGR expression, by, for example, using CRISPR technology or antisense.

In some embodiments, the GCG inhibitor or GCGR antagonist is an antisense molecule, antibody, small molecule inhibitor, peptide inhibitor, DARPin, Spiegelmer, aptamer, engineered Fn type-III domains, or a derivative thereof.

### Anti-GCGR Antibodies, Anti-GCG Antibodies, and Antibody Fragments

In some embodiments, the GCGR antagonist is an antibody or antibody fragment as disclosed in U.S. Pat. No. 8,545,847, incorporated by reference herein in its entirety. Antibodies disclosed therein are provided in Table 1.

TABLE 1

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| H4H1345N | 2 | 4 | 6 | 8 | 10 | 12 | 14 | 16 |
| H4H1617N | 18 | 20 | 22 | 24 | 26 | 28 | 30 | 32 |
| H4H1765N | 34 | 36 | 38 | 40 | 42 | 44 | 46 | 48 |
| H4H1321B | 50 | 52 | 54 | 56 | 58 | 60 | 62 | 64 |
| H4H1321P | 66 | 52 | 54 | 56 | 68 | 60 | 62 | 64 |
| H4H1327B | 70 | 72 | 74 | 76 | 78 | 80 | 82 | 84 |
| H4H1327P | 86 | 72 | 74 | 76 | 88 | 80 | 82 | 84 |
| H4H1328B | 90 | 92 | 94 | 96 | 98 | 100 | 102 | 104 |
| H4H1328P | 106 | 92 | 94 | 96 | 108 | 100 | 102 | 104 |
| H4H1331B | 110 | 112 | 114 | 116 | 118 | 120 | 122 | 124 |
| H4H1331P | 126 | 112 | 114 | 116 | 128 | 120 | 122 | 124 |
| H4H1339B | 130 | 132 | 134 | 136 | 138 | 140 | 142 | 144 |
| H4H1339P | 146 | 132 | 134 | 136 | 148 | 140 | 142 | 144 |

Additional GCGR antibodies or antibody fragments contemplated as useful herein include those disclosed in U.S. Pat. Nos. 5,770,445 and 7,947,809; European patent application EP2074149A2; EP patent EP0658200B1; U.S. patent publications 2009/0041784; 2009/0252727; and 2011/0223160; and PCT publication WO2008/036341. The patents and publications are incorporated by reference herein in their entirety.

In some embodiments, the GCG inhibitor is an antibody or antibody fragment thereof as disclosed in U.S. 2016/0075778, incorporated by reference herein in its entirety. Antibodies disclosed therein are provided in Table 2.

TABLE 2

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| H1H059P | 150 | 152 | 154 | 156 | 158 | 160 | 162 | 164 |
| H4H10223P | 166 | 168 | 170 | 172 | 174 | 176 | 178 | 180 |
| H4H10231P | 182 | 184 | 186 | 188 | 190 | 192 | 194 | 196 |
| H4H10232P | 198 | 200 | 202 | 204 | 206 | 208 | 210 | 212 |
| H4H10236P | 214 | 216 | 218 | 220 | 222 | 224 | 226 | 228 |
| H4H10237P | 230 | 232 | 234 | 236 | 238 | 240 | 242 | 244 |
| H4H10238P | 246 | 248 | 250 | 252 | 254 | 256 | 258 | 260 |
| H4H10250P | 262 | 264 | 266 | 268 | 270 | 272 | 274 | 276 |
| H4H10256P | 278 | 280 | 282 | 284 | 286 | 288 | 290 | 292 |
| H4H10270P | 294 | 296 | 298 | 300 | 302 | 304 | 306 | 308 |

Additional GCG antibodies or antibody fragments contemplated as useful herein include those disclosed in U.S. Pat. Nos. 4,206,199; 4,221,777; 4,423,034; 4,272,433; 4,407,965; 5,712,105; and PCT publications WO2007/124463 and WO2013/081993.

Antibody fragments include any fragment having the required target specificity, e.g. antibody fragments either produced by the modification of whole antibodies (e.g. enzymatic digestion), or those synthesized de novo using recombinant DNA methodologies (scFv, single domain antibodies, DVD (dual variable domain immunoglobulins), or dAbs (single variable domain antibodies)) or those identified using human phage or yeast display libraries (see, for example, McCafferty et al. (1990) Nature 348:552-554). Alternatively, antibodies can be isolated from mice producing human, human-mouse, human-rat, and human-rabbit chimeric antibodies using standard immunization and antibody isolation methods, including but not limited to making hybridomas, or using B cell screening technologies, such as SLAM. Immunoglobulin binding domains also include, but are not limited to, the variable regions of the heavy (V_{H}) or the light (V_{L}) chains of immunoglobulins. Or by immunizing people and isolating antigen positive B cells and cloning the cDNAs encoding the heavy and light chain and coexpressing them in a cell, such as CHO.

### Therapeutic Administration and Formulations

The invention provides therapeutic compositions comprising a GCGR signaling antagonist, such as the anti-GCGR antibodies or antigen-binding fragments thereof as disclosed herein. The administration of therapeutic compositions in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of antibody may vary depending upon the age and the size of a subject to be administered, target disease, conditions, route of administration, and the like. When the antibody of the present invention is used for lowering blood glucose levels associated with GCGR activity in various conditions and diseases, such as diabetes, in an adult patient, it is advantageous to intravenously administer the antibody of the present invention normally at a single dose of about 0.01 to about 30 mg/kg body weight, more preferably about 0.02 to about 7, about 0.03 to about 5, or about 0.05 to about 3 mg/kg body weight. Depending on the severity of the condition, the frequency and the duration of the treatment can be adjusted. In certain embodiments, the antibody or antigen-binding fragment thereof of the invention can be administered as an initial dose of at least about 0.1 mg to about 800 mg, about 1 to about 500 mg, about 5 to about 300 mg, or about 10 to about 200 mg, to about 100 mg, or to about 50 mg. In certain embodiments, the initial dose may be followed by administration of a second or a plurality of subsequent doses of the antibody or antigen-binding fragment thereof in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al. (1987) J. Biol. Chem. 262:4429-4432). Methods of introduction include, but are not limited to, intradermal, transdermal, intramuscular, intraperitoneal, intravenous, subcutaneous, intranasal, epidural and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

The pharmaceutical composition can be also delivered in a vesicle, in particular a liposome (see, for example, Langer (1990) Science 249:1527-1533).

In certain situations, the pharmaceutical composition can be delivered in a controlled release system. In one embodiment, a pump may be used. In another embodiment, polymeric materials can be used. In yet another embodiment, a controlled release system can be placed in proximity of the composition's target, thus requiring only a fraction of the systemic dose.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but certainly are not limited to AUTOPEN™ (Owen Mumford, Inc., Woodstock, UK), DISETRONIC™ pen (Disetronic Medical Systems, Burghdorf, Switzerland), HUMALOG MIX 75/25™ pen, HUMALOG™ pen, HUMALIN70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present invention include, but certainly are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousands Oaks, Calif.), the PENLET™ (Haselmeier, Stuttgart, Germany), the EPIPEN (Dey, L. P.) and the HUMIRA™ Pen (Abbott Labs, Abbott Park, Ill.), to name only a few.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to about 500 mg per dosage form in a unit dose; especially in the form of injection, it is preferred that the aforesaid antibody is contained in about 5 to about 100 mg and in about 10 to about 250 mg for the other dosage forms.

### Therapeutic Uses of the Antibodies

Due to their interaction with the glucagon receptor, the present antibodies are useful for lowering blood glucose levels and also for the treatment of a wide range of conditions and disorders in which blocking the interaction of glucagon with its receptor is beneficial. When therapy with the anti-GCGR antibodies of the invention is combined with an inhibitor of SLC38A5, or an inhibitor of mTOR, glucose lowering is maintained but there is no adverse effect on alpha cell hyperplasia. These disorders and conditions may be selected from any glucagon related metabolic disorder, which involves glucagon receptor signaling that results in the pathophysiology of the disorder, or in the homeostatic response to the disorder. Thus, the antibodies may find use for example to prevent, treat, or alleviate, diseases or conditions or associated symptoms or sequelae, of the endocrine system, the central nervous system, the peripheral nervous system, the cardiovascular system, the pulmonary system, and the gastrointestinal system, while reducing and or eliminating one or more of the unwanted side effects associated with the current treatments. Glucagon related metabolic disorders include, but are not limited to, type 1 and type 2 diabetes, diabetic ketoacidosis, hyperglycemia, hyperglycemic hyperosmolar syndrome, perioperative hyperglycemia, hyperglycemia in the intensive care unit patient, hyperinsulinemia, postprandial hyperglycemia, impaired fasting glucose (IFG), metabolic syndrome, hyper-/hypokalemia, poor LDL/HDL ratio, eating disorders, weight gain, obesity as a consequence of diabetes, pediatric diabetes, gestational diabetes, diabetic late complications, micro-/macroalbuminuria, nephropathy, retinopathy, neuropathy, diabetic foot ulcers, wound healing, impaired glucose tolerance (IGT), insulin resistance syndromes, syndrome X, glucagonomas, gastrointestinal disorders, obesity, diabetes as a consequence of obesity, etc. The present invention further provides; a method of treating conditions resulting from excessive glucagon in a mammal; a method of inhibiting the glucagon receptor in a mammal; a method of inhibiting a glucagon receptor mediated cellular response in a mammal, or a method of reducing the glycemic level in a mammal comprising administering to a mammal in need of such treatment a glucagon receptor-inhibiting amount of an anti-GCGR antibody or a biologically active fragment thereof.

The present antibodies are effective in lowering blood glucose, both in the fasting and the postprandial stage. In certain embodiments of the invention, the present antibodies are used for the preparation of a pharmaceutical composition for the treatment of type 2 diabetes. In yet a further embodiment of the invention the present antibodies are used for the preparation of a pharmaceutical composition for the delaying or prevention of the progression from impaired glucose tolerance to type 2 diabetes. In yet another embodiment of the invention the present antibodies are used for the preparation of a pharmaceutical composition for the delaying or prevention of the progression from non-insulin requiring diabetes to insulin requiring diabetes. In a further embodiment of the invention the present antibodies are used for the preparation of a pharmaceutical composition for the treatment of type 1 diabetes. Such treatment is normally accompanied by insulin therapy.

### Combination Therapies

Combination therapies may include an anti-hGCGR antibody or an anti-hGCG antibody and any additional therapeutic agent that may be advantageously combined with the antibody, or with a biologically active fragment of an antibody.

As noted above, inhibition of glucagon signaling often results in alpha cell (a-cell or α-cell) hyperplasia. This effect may limit the use of the glucagon signaling pathway antagonists in the clinic, such that only acute use is warranted, as opposed to chronic therapy. As such, it would be advantageous to identify an agent, such as those described herein, which prevents alpha cell hyperplasia without having a deleterious effect on the glucose lowering capabilities of the GCG or GCGR antibodies of the invention. An antagonist or inhibitor of any of the amino acid transporters described herein, as well as any mTOR inhibitor may be considered as useful agents to be combined with the GCG or GCGR antibodies. The agents described herein are amino acid transport inhibitors, such as an inhibitor of the amino acid transporter referred to herein as SLC38A5, as well as inhibitors of mTOR (e.g. rapamycin).

Furthermore, additional therapeutic agents may be employed to aid in further lowering of glucose levels, or to reduce at least one symptom in a patient suffering from a disease or condition characterized by high blood glucose levels, such as diabetes mellitus. Such an agent may be selected from, for example, a glucagon antagonist or another GCGR signaling antagonist (e.g. an anti-glucagon antibody or an anti-GCGR antibody or small molecule inhibitor of glucagon or GCGR), or may include other therapeutic moieties useful for treating diabetes, or other diseases or conditions associated with, or resulting from elevated blood glucose levels, or impaired glucose metabolism, or agents useful for treating any long term complications associated with elevated and/or uncontrolled blood glucose levels. These agents include biguanides, which decrease glucose production in the liver and increase sensitivity to insulin (e.g. metformin), or sulfonylureas, which stimulate insulin production (e.g. glyburide, glipizide). Additional treatments directed at maintaining glucose homeostasis including PPAR gamma agonists, which act as insulin sensitizers (e.g. pioglitazone, rosiglitazone); and alpha glucosidase inhibitors, which slow starch absorption and glucose production (e.g. acarbose, voglibose). Additional treatments include injectable treatments such as Exenatide® (glucagon-like peptide 1), and Symlin® (pramlintide).

In certain other embodiments, the composition may include a second agent selected from the group consisting of non-sulfonylurea secretagogues, insulin, insulin analogs, exendin-4 polypeptides, beta 3 adrenoceptor agonists, PPAR agonists, dipeptidyl peptidase IV inhibitors, statins and statin-containing combinations, cholesterol absorption inhibitors, LDL-cholesterol antagonists, cholesteryl ester transfer protein antagonists, endothelin receptor antagonists, growth hormone antagonists, insulin sensitizers, amylin mimetics or agonists, cannabinoid receptor antagonists, glucagon-like peptide-1 agonists, melanocortins, melanin-concentrating hormone receptor agonists, SNRIs, and protein tyrosine phosphatase inhibitors.

In certain other embodiments, combination therapy may include administration of a second agent to counteract any potential side effect(s) resulting from administration of an antibody of the invention, if such side effect(s) occur. For example, in the event that any of the anti-GCGR antibodies increases lipid or cholesterol levels, it may be beneficial to administer a second agent to lower lipid or cholesterol levels, using an agent such as a HMG-CoA reductase inhibitor (for example, a statin such as atorvastatin, (LIPITOR®), fluvastatin (LESCOL®), lovastatin (MEVACOR®), pitavastatin (LIVALO®), pravastatin (PRAVACHOL®), rosuvastatin (CRESTOR®) and simvastatin (ZOCOR®). Alternatively, the antibodies of the invention may be combined with an agent such as VYTORIN®, which is a preparation of a statin and another agent—such as ezetimibe/simvastatin.

In certain embodiments, it may be beneficial to administer the antibodies of the invention in combination with any one or more of the following: (1) niacin, which increases lipoprotein catabolism; (2) fibrates or amphipathic carboxylic acids, which reduce low-density lipoprotein (LDL) level, improve high-density lipoprotein (HDL) and TG levels, and reduce the number of non-fatal heart attacks; and (3) activators of the LXR transcription factor that plays a role in cholesterol elimination such as 22-hydroxycholesterol, or a statin with a bile resin (e.g., cholestyramine, colestipol, colesevelam), a fixed combination of niacin plus a statin (e.g., niacin with lovastatin); or with other lipid lowering agents such as omega-3-fatty acid ethyl esters (for example, omacor).

Furthermore, the additional therapeutic agent can be one or more other inhibitors of glucagon or GCGR, as well as inhibitors of other molecules, such as angiopoietin-like protein 3 (ANGPTL3), angiopoietin-like protein 4 (ANGPTL4), angiopoietin-like protein 5 (ANGPTL5), angiopoietin-like protein 6 (ANGPTL6), which are involved in lipid metabolism, in particular, cholesterol and/or triglyceride homeostasis. Inhibitors of these molecules include small molecules and antibodies that specifically bind to these molecules and block their activity.

In certain embodiments, it may be beneficial to administer the antibodies provided herein in combination with an antibody that acts to lower lipid or cholesterol levels, such as, but not limited to, for example, any anti-PCSK9 (proprotein convertase subtilisin/kexin type 9) antibody, such as those described in US2010/0166768. Other anti-PCSK9 antibodies are described in US2010/0040611, US2010/0041102, US2010/0040610, US2010/0113575, US2009/0232795, US2009/0246192, US2010/0233177, US2009/0142352, US2009/0326202, US2010/0068199, US2011/0033465, US2011/0027287, US2010/0150937, US2010/0136028 and WO2009/055783.

In certain embodiments, it may be beneficial to administer the anti-GCGR or anti-GCG antibodies provided herein in combination with a nucleic acid that inhibits the activity of PCSK9 (proprotein convertase subtilisin/kexin type 9), such as an antisense molecule, a double stranded RNA, or a siRNA molecule. Exemplary nucleic acid molecules that inhibit the activity of PCSK9 are described in US2011/0065644, US2011/0039914, US2008/0015162 and US2007/0173473.

The additional therapeutically active component(s) may be administered prior to, concurrent with, or after the administration of the anti-GCGR antibody or the anti-GCG antibody. For purposes of the present disclosure, such administration regimens are considered the administration of an anti-GCGR antibody or an anti-GCG antibody “in combination with” one or more therapeutically active components.

### EXAMPLES

### Example 1: Amino Acid Transporter Slc38a5 Mediates Glucagon Receptor Inhibition-Induced Pancreatic α-Cell Hyperplasia in Mice

Glucagon supports glucose homeostasis by stimulating hepatic gluconeogenesis, in part by promoting the uptake and conversion of amino acids into gluconeogenic precursors. Genetic disruption or pharmacologic inhibition of glucagon signaling results in elevated plasma amino acids, and compensatory glucagon hypersecretion involving expansion of pancreatic α-cell mass. Regulation of pancreatic alpha- and beta-cell growth has drawn a lot of attention because of potential therapeutic implications. Recent findings indicate that hyperaminoacidemia triggers pancreatic alpha-cell proliferation via an mTOR-dependent pathway. The experiments provided below demonstrate that glucagon pathway blockade selectively increases expression of the sodium-coupled neutral amino acid transporter Slc38a5 in a subset of highly proliferative alpha-cells, and that Slc38a5 is critical for the pancreatic response to glucagon pathway blockade; most notably, mice deficient in Slc38a5 exhibit markedly decreased alpha-cell hyperplasia to glucagon pathway blockade-induced hyperaminoacidemia. These results show that Slc38a5 is a key component of the feedback circuit between glucagon receptor signaling in the liver and amino acid-dependent regulation of pancreatic alpha-cell mass in mice.

### EXPERIMENTAL PROCEDURES

### In Vivo Studies

All procedures were conducted in compliance with protocols approved by the Institutional Animal Care and Use Committee of Regeneron Pharmaceuticals. The in vitro and in vivo characteristics of REGN1193 (H4H1327P), a potent monoclonal GCGR blocking antibody has been previously described (See U.S. Pat. No. 8,545,847). This antibody was used as the exemplary glucagon signaling pathway antagonist in all studies in this report (See the HVCR/LCVR amino acid sequence pair of SEQ ID NOs: 86/88 in the present application. See also the corresponding heavy chain complementarity determining regions (HCDRs 1-2-3 of SEQ ID NOs: 72-74-76) and the corresponding light chain complementarity determining regions (LCDRs 1-2-3 of SEQ ID NOs: 80-82-84). The GCGR antibody and isotype control antibody were diluted with sterile PBS.

### C57BL/6 Mice

C57BL/6 mice (males, 10 weeks of age, Taconic) were housed (5 mice per cage) in a controlled environment (12-h light/dark cycle, 22±1° C., 60-70% humidity) and fed ad libitum with standard chow (Purina Laboratory Rodent Diet 5001, LabDiet). Mice were assigned to study groups (n=10) based on baseline blood glucose levels. Mice were injected subcutaneously (sc) with GCGR antibody (3 mg/kg) or the control antibody (3 mg/kg) alone or in combination with rapamycin (10 mg/kg) or the vehicle. For the duration of the study, GCGR or control antibody was administered once weekly, whereas rapamycin or the vehicle was administered daily. Blood was collected from the tail for glucose measurements without fasting. Plasma samples were collected via submandibular bleeds at baseline and on day 21 after dosing for analysis of hormones and total amino acid levels. Seven days after the last antibody administration (day 21), mice were sacrificed, organs weighed and pancreata collected.

### Slc38a5^{-/-} Mice

Mice deficient in Slc38a5 (100% C57BL/6NTac) were generated by homologous recombination using Regeneron's VelociGene technology (Valenzuela et al., 2003, Nat Biotechnol 21:652-659). VelociGene allele identification number is VG15016. Both male and female mice were used for the study. Mice were fed ad libitum with standard chow (Purina Laboratory Rodent Diet 5001, LabDiet). Slc38a5^{-/-} mice and wildtype littermates were dosed with GCGR or control antibody (10 mg/kg; n=8) weekly for 20 days. Samples of blood and plasma as well as organ weights and pancreata were collected as outlined above.

### Islet Isolation and Enrichment of Islet α-Cells

Mouse islets were isolated by density gradient separation after perfusing pancreas with Liberase TL (Roche, Indianapolis) through the common bile duct. Following a 13 min digestion at 37° C., the pancreas solution was washed and filtered through a 400-μm wire mesh strainer and islets were separated by Histopaque gradient centrifugation (Sigma-Aldrich). Isolated islets were cultured overnight in RPMI-1640 medium supplemented with 10% (v/v) FBS, 10 mM HEPES, 50 μM β-mercaptoethanol, 1.0 mM sodium-pyruvate, 100 U/mL penicillin and 100 μg/mL streptomycin at 37° C. with a 5% CO₂ in air atmosphere.

### RNA Preparation

Total RNA was purified from all samples using MagMAX™-96 for Microarrays Total RNA Isolation Kit (Ambion by Life Technologies) according to manufacturer's specifications. Genomic DNA was removed using MagMAX™Turbo™DNase Buffer and TURBO DNase from the MagMAX kit listed above (Ambion by Life Technologies). mRNA was purified from total RNA using Dynabeads® mRNA Purification Kit (Invitrogen). Strand-specific RNA-seq libraries were prepared using ScriptSeq™ mRNA-Seq Library Preparation Kit (Epicentre). Twelve-cycle PCR was performed to amplify libraries. Sequencing was performed on Illumina HiSeq®2000 (Illumina) by multiplexed single-read run with 33 cycles.

### RNAseq Read Mapping and Statistical Analysis of Differentially Expressed RNA

Raw sequence data (BCL files) were converted to FASTQ format via Illumina Casava 1.8.2. Reads were decoded based on their barcodes and read quality was evaluated with FastQC (http://www.bioinformatics.babraham.ac.uk/projects/fastqc/). Reads were mapped to the mouse transcriptome (NCBI GRCh37) using CLC Genomics Workbench Version 7.0 (CLCbio) or TopHat2 (Version 2.0.10) allowing two mismatches. Reads mapped to the exons of a gene were summed at the gene level. To identify differentially expressed genes, the statistical significance (p-value) of the differential expressions was assessed with DESeq package (version 1.6 or 2.0). At the end, significantly perturbed genes with fold changes no less than 1.5 in either up or down direction and with p-values of at least 0.01 or FDR <0.05 were selected.

### Immunohistochemistry and Immunofluorescence

Pancreas α- and β-cell mass was measured as described elsewhere (Okamato et al., 2015, Endocrinology 156:2781-2794). α- and PP cell mass were determined using anti-somatostatin antibody (Sigma-Aldrich; #SAB4502861) and anti-pancreatic polypeptide antibody (Sigma-Aldrich; #SAB2500747). For quantifying Slc38a5 positive α-cells, pancreas sections were stained with human anti-glucagon and rabbit anti-Slc38a5 (Abcam; #ab72717) primary antibodies, and Alexa Fluor 488 labeled donkey anti-rabbit (Jackson ImmunoResearch; #711-545-152) and Alexa Fluor 594 donkey anti-human (Jackson ImmunoResearch; #709-585-149) secondary antibodies. For quantifying proliferating cells, Alexa Fluor 647 labeled anti-Ki67 (Abcam; #ab194724) was used in combination with anti-glucagon and anti-Slc38a5 antibodies. Slides were scanned using a Zeiss Axio Scan.Z1 slide scanner (Zeiss) and the images were analyzed using the Halo (Indica Labs).

### RNA In Situ Hybridization

Formalin fixed mouse pancreatic sections were permeabilized and hybridized with custom made mRNA probes to glucagon and mouse Slc38a5 (ACD BIO). Following probe hybridization and amplification, Slc38a5 mRNA was detected using red chromogen and glucagon mRNA with green chromogen (RNAscope 2.5 HD Duplex Reagent Kit) and images were obtained using a Zeiss Axio Scan.Z1 slide scanner (Zeiss).

### Blood Chemistry

Blood glucose was determined using ACCU-CHEK® Compact Plus (Roche Diagnostics). Plasma glucagon and insulin levels were determined using glucagon and mouse insulin ELISA (Mercodia). Plasma total amino acid levels were measured using L-Amino Acid Quantification Kit (Sigma-Aldrich).

### Generation of Slc38a5 Deficient αTC1-6 Cells

Slc38a5 KO αTC1 clones were generated using CRISPR-Cas9 techniques. Target sequences (sgRNAs) were designed using an online sgRNA design tool from DNA2.0 (DNA2.0). The sgRNAs were cloned into the pSpCas9-2A-GFP-Puro vector using BsmBl (NEB) and αTC1 cells were transfected with Lipofectamine 2000 according to the manufacturers' instructions (Life Technologies). Clones with deletion mutations were verified by PCR amplifications, TaqMan analysis and RNA sequencing. A clone with reduced expression of Slc38a5 (Slc38a5 KD) was used in cell proliferation assay to test the effect of amino acids on cell growth.

### Cell Proliferation

Both WT and Slc38a5 KD αTC1/6 cells were prepared in an assay medium (MEM from Gibco), supplemented with 5% fetal bovine serum (FBS), 10 mM HEPES, and 100 IU/ml penicillin and 100 μg/ml streptomycin) and seeded on 96-well plates at a density of ˜15,000 cells per well in 4-6 replicates. Selected amino acids were added to the final concentration of 4 mM. Cell proliferations were measured every day using the CellTiter 96 Aqueous Cell Proliferation Assay (Promega) according to the manufacturer's instructions. Briefly, 20 μl of assay reagent was added to each well and the cell plates were incubated in a tissue culture incubator for 2.5 hours. Absorbance was measured at 490 nm on a plate reader SpectraMax Plus (Molecular Devices). Values were corrected for background absorbance using the averages of the wells containing assay medium only and plotted in Prism (GraphPad).

### Data Analyses

All data are mean±SEM. Statistical analyses were performed utilizing GraphPad software Prism 6.0. All parameters were analyzed by student's t-test, one-way ANOVA or two-way ANOVA; a threshold of P<0.05 was considered statistically significant. If a significant F ratio was obtained with one or two-way ANOVA, post hoc analysis was conducted with Bonferroni post-tests.

### Results

Reduced Hepatic Amino Acid Transporter and Metabolism Gene Expression in Mice with Impaired Glucagon Receptor Signaling

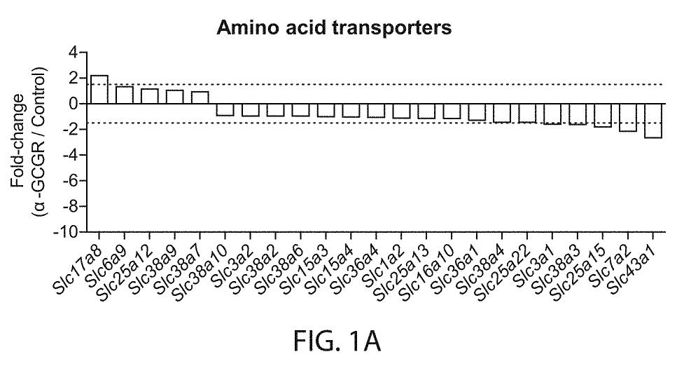

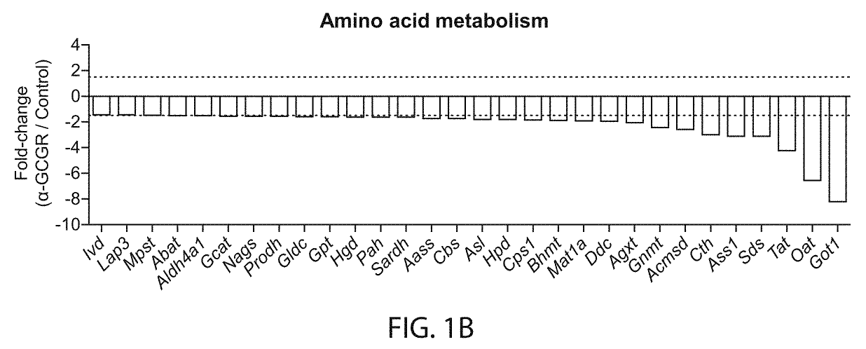

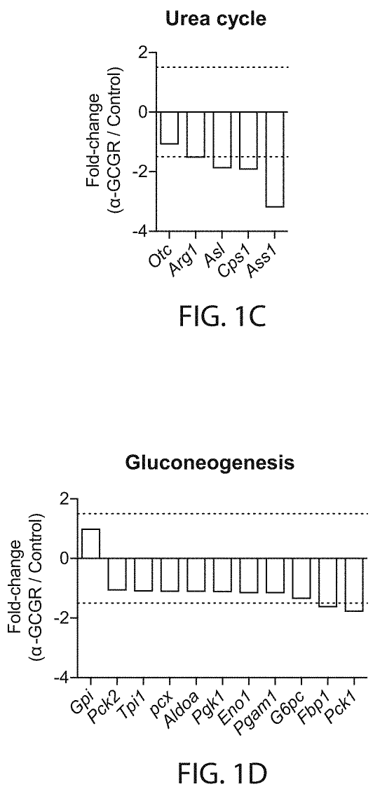

A key role of glucagon is to promote the uptake and metabolism of amino acids in the liver to provide gluconeogenic precursors. To further explore this pathway, a recently described fully human GCGR-blocking antibody (Okamato et al, 2015, Endocrinol. 156:2781-2794; See also, U.S. Pat. No. 8,545,847) derived using VelocImmune technology (Macdonald et al., 2014, PNAS 111:5147-5152; Murphy et al., 2014, PNAS 111:5153-5158) was used in the experiments provided herein. The expression of the plasma membrane amino acid transporters Slc38a3, Slc38a4, Slc7a2 and Slc3a1 was found to be reduced by 30-50% in livers of mice treated with the GCGR-antibody (15 mg/kg) for 21 days (FIG. 1A and Table 3). In addition, the expression of the mitochondrial amino acid transporters Slc25a15 and Slc25a22 was significantly reduced. Consistent with previous reports (Gu et al., 2011, J. Pharmacol Exp Ther 338:70-81; Mu et al., 2012, PLOS ONE 7:e49572; Solloway et al., 2015, Cell Reports 12:495-510), reduced expression of genes involved in amino acid metabolism, elimination of nitrogen in the urea cycle, and gluconeogenesis (FIGS. 1B-1D and Table 4) was detected. These data explain how inhibition of GCGR signaling results in reduced hepatic glucose output and blood glucose levels. As shown previously (Mu et al., 2012, PLOS ONE 7:349572; Okamoto et al., 2015, Endocrinol. 156:2781-2794; Solloway et al., 2015, Cell Reports 12:495-510), and confirmed later in this study, these findings provide a mechanism for the elevated circulating amino acid levels that are observed in mice and monkeys with impaired GCGR signaling.

TABLE 3
List of amino acid transporter genes in liver of GCGR antibody treated mice and
Gcgr KO mice

| | | | | | GCGR | GCGR | | |
|---|---|---|---|---|---|---|---|---|
| | Control* | α-GCGR* | Fold | P | WT | KO | Fold | P |
| Symbol/Description | RPKM | RPKM | change | value | RPKM | RPKM | change | value |
| Slc17a8/solute | 1.61 | 4.18 | 2.32 | 1.70E−05 | 2.53 | 7.37 | 2.65 | 6.20E−04 |
| carrier family 17 | | | | | | | | |
| (sodium-dependent | | | | | | | | |
| inorganic | | | | | | | | |
| phosphate | | | | | | | | |
| cotransporter), | | | | | | | | |
| member 8 | | | | | | | | |
| Slc6a9/solute | 6.25 | 10.29 | 1.45 | 7.20E−03 | 5.55 | 8.39 | 1.38 | 6.10E−03 |
| carrier family 6 | | | | | | | | |
| (neurotransmitter | | | | | | | | |
| transporter, | | | | | | | | |
| glycine), member 9 | | | | | | | | |
| Slc25a12/solute | 3.32 | 4.68 | 1.27 | 6.90E−02 | 4.88 | 5.79 | 1.08 | 4.90E−01 |
| carrier family 25 | | | | | | | | |
| (mitochondrial | | | | | | | | |
| carrier, Aralar), | | | | | | | | |
| member 12 | | | | | | | | |
| Slc38a9/solute | 1.14 | 1.48 | 1.17 | 2.20E−01 | 1.41 | 1.63 | 1.05 | 6.00E−01 |
| carrier family 38, | | | | | | | | |
| member 9 | | | | | | | | |
| Slc38a7/solute | 3.75 | 4.41 | 1.05 | 8.20E−01 | 2.34 | 3.62 | 1.41 | 1.10E−02 |
| carrier family 38, | | | | | | | | |
| member 7 | | | | | | | | |
| Slc38a10/solute | 18.13 | 19.58 | −1.04 | 6.20E−01 | 13.88 | 19.73 | 1.30 | 1.80E−01 |
| carrier family 38, | | | | | | | | |
| member 10 | | | | | | | | |
| Slc3a2/solute | 13.87 | 14.56 | −1.08 | 4.90E−01 | 7.41 | 7.98 | −1.02 | 8.70E−01 |
| carrier family 3 | | | | | | | | |
| (activators of | | | | | | | | |
| dibasic and neutral | | | | | | | | |
| amino acid | | | | | | | | |
| transport), member 2 | | | | | | | | |
| Slc38a2/solute | 16.94 | 17.59 | −1.08 | 5.30E−01 | 19.69 | 18.15 | −1.19 | 2.80E−01 |
| carrier family 38, | | | | | | | | |
| member 2 | | | | | | | | |
| Slc38a6/solute | 1.67 | 1.72 | −1.09 | 6.60E−01 | 1.58 | 1.53 | −1.12 | 5.30E−01 |
| carrier family 38, | | | | | | | | |
| member 6 | | | | | | | | |
| Slc15a3/solute | 1.18 | 1.15 | −1.12 | 7.00E−01 | 0.90 | 0.96 | −1.04 | 9.70E−01 |
| carrier family 15, | | | | | | | | |
| member 3 | | | | | | | | |
| Slc15a4/solute | 5.67 | 5.47 | −1.15 | 3.00E−01 | 4.87 | 4.12 | −1.30 | 3.50E−02 |
| carrier family 15, | | | | | | | | |
| member 4 | | | | | | | | |
| Slc36a4/solute | 1.42 | 1.34 | −1.18 | 5.20E−01 | 1.81 | 1.89 | −1.04 | 7.50E−01 |
| carrier family 36 | | | | | | | | |
| (proton/amino acid | | | | | | | | |
| symporter), | | | | | | | | |
| member 4 | | | | | | | | |
| Slc1a2/solute | 16.80 | 14.89 | −1.25 | 3.90E−02 | 29.06 | 16.08 | −2.00 | 4.90E−12 |
| carrier family 1 | | | | | | | | |
| (glial high affinity | | | | | | | | |
| glutamate | | | | | | | | |
| transporter), | | | | | | | | |
| member 2 | | | | | | | | |
| Slc25a13/solute | 85.92 | 76.40 | −1.27 | 2.20E−02 | 87.97 | 78.36 | −1.23 | 3.10E−02 |
| carrier family 25 | | | | | | | | |
| (mitochondrial | | | | | | | | |
| carrier, adenine | | | | | | | | |
| nucleotide | | | | | | | | |
| translocator), | | | | | | | | |
| member 13 | | | | | | | | |
| Slc16a10/solute | 17.27 | 14.93 | −1.28 | 1.60E−02 | 17.42 | 13.47 | −1.41 | 5.50E−03 |
| carrier family 16 | | | | | | | | |
| (monocarboxylic | | | | | | | | |
| acid transporters), | | | | | | | | |
| member 10 | | | | | | | | |
| Slc36a1/solute | 4.92 | 3.90 | −1.41 | 6.60E−03 | 4.60 | 3.43 | −1.47 | 1.10E−03 |
| carrier family 36 | | | | | | | | |
| (proton/amino acid | | | | | | | | |
| symporter), | | | | | | | | |
| member 1 | | | | | | | | |
| Slc38a4/solute | 364.79 | 261.42 | −1.56 | 2.00E−05 | 414.53 | 234.17 | −1.96 | 1.60E−11 |
| carrier family 38, | | | | | | | | |
| member 4 | | | | | | | | |
| Slc25a22/solute | 20.20 | 14.40 | −1.56 | 5.00E−05 | 19.83 | 14.65 | −1.49 | 1.40E−02 |
| carrier family 25 | | | | | | | | |
| (mitochondrial | | | | | | | | |
| carrier, glutamate), | | | | | | | | |
| member 22 | | | | | | | | |
| Slc3a1/solute | 15.34 | 9.78 | −1.72 | 1.00E−03 | 15.18 | 9.84 | −1.69 | 5.40E−05 |
| carrier family 3, | | | | | | | | |
| member 1 | | | | | | | | |
| Slc38a3/solute | 448.85 | 285.49 | −1.75 | 7.70E−08 | 556.80 | 320.95 | −1.89 | 5.10E−07 |
| carrier family 38, | | | | | | | | |
| member 3 | | | | | | | | |
| Slc25a15/solute | 86.81 | 50.85 | −1.92 | 7.00E−10 | 74.36 | 49.99 | −1.64 | 5.70E−07 |
| carrier family 25 | | | | | | | | |
| (mitochondrial | | | | | | | | |
| carrier ornithine | | | | | | | | |
| transporter), | | | | | | | | |
| member 15 | | | | | | | | |
| Slc7a2/solute | 138.40 | 67.24 | −2.27 | 5.80E−15 | 191.46 | 55.48 | −3.85 | 7.90E−11 |
| carrier family 7 | | | | | | | | |
| (cationic amino acid | | | | | | | | |
| transporter, y+ | | | | | | | | |
| system), member 2 | | | | | | | | |
| Slc43a1/solute | 2.11 | 0.83 | −2.78 | 5.30E−03 | 2.16 | 0.62 | −3.70 | 1.00E−03 |
| carrier family 43, | | | | | | | | |
| member 1 | | | | | | | | |

*15 mg/kg Control or α-GCGR

TABLE 4
List of amino acid metabolism, urea cycle, and gluconeogenesis genes in liver of GCGR
antibody treated mice and Gcgr KO mice

| | | | | | | | | |
|---|---|---|---|---|---|---|---|---|
| | | α- | | | GCGR | GCGR | | |
| | Control* | GCGR* | Fold | P | WT | KO | Fold | P |
| Symbol/Description | RPKM | RPKM | change | value | RPKM | RPKM | change | value |

Amino acid metabolism

| | | | | | | | | |
|---|---|---|---|---|---|---|---|---|
| Ivd/isovaleryl coenzyme A | 72.09 | 51.86 | −1.56 | 2.60E−05 | 88.39 | 63.24 | −1.54 | 1.60E−05 |
| dehydrogenase | | | | | | | | |
| Lap3/leucine | 152.58 | 108.58 | −1.56 | 1.70E−05 | 146.35 | 107.58 | −1.49 | 6.20E−05 |
| aminopeptidase 3 | | | | | | | | |
| Mpst/mercaptopyruvate | 25.01 | 17.32 | −1.61 | 9.90E−04 | 13.34 | 10.74 | −1.37 | 7.30E−02 |
| sulfurtransferase | | | | | | | | |
| Abat/4-aminobutyrate | 62.98 | 43.18 | −1.64 | 1.80E−06 | 70.37 | 42.91 | −1.79 | 2.70E−09 |
| aminotransferase | | | | | | | | |
| Aldh4a1/aldehyde | 94.01 | 64.72 | −1.64 | 1.90E−06 | 72.17 | 50.04 | −1.59 | 3.20E−06 |
| dehydrogenase 4 family, | | | | | | | | |
| member A1 | | | | | | | | |
| Gcat/glycine C- | 27.75 | 18.88 | −1.67 | 2.10E−03 | 9.94 | 8.69 | −1.25 | 1.00E−01 |
| acetyltransferase (2- | | | | | | | | |
| amino-3-ketobutyrate- | | | | | | | | |
| coenzyme A ligase) | | | | | | | | |
| Nags/N-acetylglutamate | 20.46 | 13.94 | −1.67 | 2.80E−05 | 15.91 | 15.68 | −1.11 | 4.20E−01 |
| synthase | | | | | | | | |
| Prodh/proline | 132.94 | 89.26 | −1.67 | 8.40E−07 | 155.42 | 124.71 | −1.37 | 2.20E−03 |
| dehydrogenase | | | | | | | | |
| Gldc/glycine | 51.16 | 33.42 | −1.72 | 5.20E−04 | 31.36 | 21.42 | −1.61 | 1.90E−06 |
| decarboxylase | | | | | | | | |
| Gpt/glutamic pyruvic | 158.76 | 103.74 | −1.72 | 1.40E−07 | 113.86 | 87.31 | −1.43 | 2.80E−03 |
| transaminase, soluble | | | | | | | | |
| Hgd/homogentisate 1,2- | 171.97 | 109.59 | −1.75 | 7.50E−08 | 190.21 | 113.62 | −1.85 | 8.80E−10 |
| dioxygenase | | | | | | | | |
| Pah/bhenylalanine | 365.20 | 232.16 | −1.75 | 3.30E−08 | 526.32 | 250.83 | −2.33 | 2.00E−11 |
| hydroxylase | | | | | | | | |
| Sardh/sarcosine | 192.78 | 122.51 | −1.75 | 3.80E−05 | 169.77 | 122.67 | −1.52 | 8.90E−05 |
| dehydrogenase | | | | | | | | |
| Aass/aminoadipate- | 100.22 | 60.38 | −1.85 | 7.50E−09 | 101.49 | 62.08 | −1.79 | 1.90E−09 |
| semialdehyde synthase | | | | | | | | |
| Cbs/cystathionine beta- | 208.90 | 126.52 | −1.85 | 3.80E−06 | 169.55 | 100.72 | −1.85 | 6.70E−10 |
| synthase | | | | | | | | |
| Asl/argininosuccinate | 261.09 | 151.51 | −1.92 | 1.40E−10 | 324.32 | 252.96 | −1.41 | 1.80E−02 |
| lyase | | | | | | | | |
| Hpd/4- | 1493.09 | 874.17 | −1.92 | 8.80E−11 | 1850.85 | 1031.93 | −1.96 | 3.90E−12 |
| hydroxyphenylpyruvic | | | | | | | | |
| acid dioxygenase | | | | | | | | |
| Cps1/carbarnoyl- | 734.83 | 419.74 | −1.96 | 7.80E−11 | 770.39 | 586.57 | −1.43 | 3.20E−03 |
| phosphate synthetase 1 | | | | | | | | |
| Bhmt/betaine- | 178.08 | 98.73 | −2.00 | 5.80E−03 | 141.54 | 76.11 | −2.04 | 5.30E−06 |
| homocysteine | | | | | | | | |
| methyltransferase | | | | | | | | |
| Mat1a/methionine | 953.63 | 525.98 | −2.04 | 8.80E−12 | 934.54 | 659.51 | −1.56 | 2.00E−04 |
| adenosyltransferase I, | | | | | | | | |
| alpha | | | | | | | | |
| Ddc/dopa decarboxylase | 28.11 | 15.03 | −2.08 | 4.90E−05 | 15.11 | 10.26 | −1.61 | 3.00E−01 |
| Agxt/alanine-glyoxylate | 215.39 | 111.91 | −2.17 | 1.10E−13 | 186.18 | 71.35 | −2.86 | 5.30E−21 |
| aminotransferase | | | | | | | | |
| Gnmt/glycine N- | 1034.56 | 449.55 | −2.56 | 1.30E−18 | 542.93 | 260.85 | −2.27 | 2.20E−07 |
| methyltransferase | | | | | | | | |
| Acmsd/amino | 9.41 | 3.90 | −2.70 | 7.00E−13 | 8.93 | 3.84 | −2.56 | 1.50E−03 |
| carboxymuconate | | | | | | | | |
| semialdehyde | | | | | | | | |
| decarboxylase | | | | | | | | |
| Cth/cystathionase | 388.14 | 139.77 | −3.12 | 4.40E−27 | 435.24 | 144.73 | −3.33 | 1.00E−08 |
| (cystathionine gamma- | | | | | | | | |
| lyase) | | | | | | | | |
| Ass1/argininosuccinate | 549.56 | 190.00 | −3.23 | 4.30E−15 | 381.55 | 123.22 | −3.45 | 3.50E−12 |
| synthetase 1 | | | | | | | | |
| Sds/serine dehydratase | 114.64 | 39.47 | −3.23 | 7.50E−26 | 76.38 | 15.58 | −5.26 | 8.90E−05 |
| Tat/tyrosine | 783.84 | 197.67 | −4.35 | 6.90E−16 | 1065.04 | 427.56 | −2.70 | 2.10E−03 |
| aminotransferase | | | | | | | | |
| Oat/ornithine | 388.55 | 66.76 | −6.67 | 1.50E−66 | 498.47 | 58.16 | −9.09 | 1.30E−29 |
| aminotransferase | | | | | | | | |
| Got1/glutamate | 74.02 | 9.98 | −8.33 | 5.70E−73 | 80.21 | 11.96 | −7.14 | 1.30E−08 |
| oxaloacetate | | | | | | | | |
| transaminase 1, soluble | | | | | | | | |

Urea cycle

| | | | | | | | | |
|---|---|---|---|---|---|---|---|---|
| Otc/Ornithine | 297.02 | 292.99 | −1.12 | 2.60E−01 | 395.46 | 301.90 | −1.43 | 2.40E−04 |
| transcarbamylase | | | | | | | | |
| Arg1/Arginase, liver | 1372.89 | 969.96 | −1.56 | 3.20E−04 | 2068.48 | 1649.36 | −1.37 | 8.30E−04 |
| Asl/Argininosuccinic acid | 261.09 | 151.51 | −1.92 | 1.40E−10 | 324.32 | 252.96 | −1.41 | 1.80E−02 |
| lyase | | | | | | | | |
| Cps1/Carbamoylphophate | 734.83 | 419.74 | −1.96 | 7.80E−11 | 770.39 | 586.57 | −1.43 | 3.20E−03 |
| synthetase I | | | | | | | | |
| Ass1/Argininosuccinic | 549.56 | 190.00 | −3.23 | 4.30E−15 | 381.55 | 123.22 | −3.45 | 3.50E−12 |
| acid synthetase | | | | | | | | |

Gluconeogenesis

| | | | | | | | | |
|---|---|---|---|---|---|---|---|---|
| Gpi1/phosphohexose | 14.41 | 16.58 | 1.03 | 9.20E−01 | 17.42 | 26.76 | 1.40 | 3.00E−02 |
| isomerase | | | | | | | | |
| Tpi1/triose phosphate | 46.77 | 46.09 | −1.14 | 2.00E−01 | 47.69 | 54.65 | 1.05 | 6.70E−01 |
| isomerase | | | | | | | | |
| pcx/Pyruvate carboxylase | 70.14 | 68.44 | −1.15 | 1.30E−01 | 64.16 | 77.87 | 1.11 | 4.00E−01 |
| Aldoa/aldolase | 16.56 | 16.24 | −1.15 | 2.90E−01 | 19.95 | 20.30 | −1.08 | 5.00E−01 |
| Pgk1/phosphoglycerate | 2.01 | 1.91 | −1.16 | 5.50E−01 | 2.11 | 1.47 | −1.56 | 1.80E−02 |
| kinase | | | | | | | | |
| Eno1/enolase | 16.09 | 14.96 | −1.20 | 2.50E−01 | 16.67 | 19.26 | 1.05 | 5.90E−01 |
| Pgam1/phosphoglycerate | 2.30 | 2.12 | −1.20 | 3.20E−01 | 2.32 | 2.27 | −1.12 | 5.20E−01 |
| mutase | | | | | | | | |
| G6pc/glucose 6- | 50.99 | 40.65 | −1.39 | 1.20E−01 | 49.02 | 47.49 | −1.12 | 6.20E−01 |
| phosphatase | | | | | | | | |
| Fbp1/frutose 1,6 | 539.78 | 365.35 | −1.67 | 6.50E−07 | 546.80 | 350.02 | −1.72 | 5.80E−08 |
| bisphophatase | | | | | | | | |
| Pck1/PEP | 804.63 | 490.87 | −1.82 | 3.30E−08 | 1472.23 | 393.20 | −4.17 | 4.30E−17 |
| carboxykinase1, cytosolic | | | | | | | | |

*15 mg/kg Control or α-GCGR

Increased Expression of the Amino Acid Transporter Slc38a5 in α-Cells of Mice with Inhibited GCGR Signaling

FIG. 2A shows changes in gene expression in isolated pancreatic islets from mice treated for 21 days with GCGR or control antibody at 10 mg/kg. Interestingly, the most upregulated gene is Slc38a5, which encodes a sodium-coupled neutral amino acid transporter with preference for L-glutamine, L-histidine, L-alanine and L-asparagine (Nakanishi et al., 2001, Am. J. Physiol Cell Physiol 281:C1757-1768). Table 5 shows the absolute expression levels and fold-change in expression for all significantly regulated genes in GCGR-antibody treated mice. Surprisingly, despite mouse pancreatic islets expressing 40 different amino acid transporters, only Slc38a5 expression was found to be regulated by the GCGR-antibody treatment (FIG. 2B). RNA in situ hybridization (RNA ISH) simultaneously using probes to Gcg and Slc38a5 confirmed upregulation of Slc38a5 mRNA expression in α-cells from GCGR-antibody treated mice (FIG. 6). Immunostaining of pancreas sections from mice treated with GCGR-antibody showed that Slc38a5 was detected in a subset of glucagon-positive cells (FIGS. 2C, 2D). The Slc38a5 expression was confined to the plasma membrane of the α-cells (FIGS. 2C, 2D and 7A) and did not associate with the lysosomes (FIG. 7B). These data show that inhibition of glucagon signaling is associated with increased expression of the amino acid transporter Slc38a5 in the plasma membrane of a subset of α-cells in mice.

TABLE 5
Complete list of significantly regulated genes in islets of GCGR antibody
treated mice and in α-cell enriched fraction of Gcgr KO mice

| | Control* | α-GCGR* | Fold | | |
|---|---|---|---|---|---|
| Symbol | RPKM | RPKM | change | P-value | Description |
| Slc38a5 | 3.05 | 52.26 | 5.54 | 7.43E−40 | solute carrier family 38, |
| | | | | | member 5 |
| Ttr | 777.89 | 5356.49 | 4.27 | 1.09E−37 | transthyretin |
| Gcg | 12299.88 | 63254.16 | 3.55 | 1.54E−28 | glucagon |
| Gpx3 | 152.69 | 638.48 | 2.86 | 2.49E−18 | glutathione peroxidase 3 |
| Pdk4 | 7.76 | 25.17 | 2.83 | 1.91E−28 | pyruvate dehydrogenase |
| | | | | | kinase, isoenzyme 4 |
| Serpine2 | 2.92 | 10.10 | 2.80 | 2.12E−22 | serine (or cysteine) |
| | | | | | peptidase inhibitor, clade E, |
| | | | | | member 2 |
| Gfra3 | 1.84 | 8.18 | 2.71 | 3.14E−15 | glial cell line derived |
| | | | | | neurotrophic factor family |
| | | | | | receptor alpha 3 |
| Ndst4 | 1.95 | 6.30 | 2.71 | 1.82E−22 | N-deacetylase/N- |
| | | | | | sulfotransferase (heparin |
| | | | | | glucosaminyl) 4 |
| Avpr1b | 1.24 | 4.45 | 2.54 | 1.11E−14 | arginine vasopressin |
| | | | | | receptor 1B |
| Ifih1 | 4.17 | 10.68 | 2.33 | 4.38E−20 | interferon induced with |
| | | | | | helicase C domain 1 |
| Wnk3 | 3.22 | 9.33 | 2.33 | 6.63E−14 | WNK lysine deficient protein |
| | | | | | kinase 3, pseudogene |
| Vwde | 1.50 | 5.37 | 2.23 | 1.59E−10 | von Willebrand factor D and |
| | | | | | EGF domains |
| Kcnk3 | 2.96 | 8.14 | 2.23 | 1.19E−12 | potassium channel, |
| | | | | | subfamily K, member 3 |
| Eef1a2 | 10.14 | 35.85 | 2.18 | 2.49E−09 | eukaryotic translation |
| | | | | | elongation factor 1 alpha 2 |
| Sftpd | 1.38 | 4.65 | 2.07 | 3.60E−08 | surfactant associated protein D |
| Peg10 | 5.90 | 17.17 | 2.06 | 1.19E−08 | paternally expressed 10 |
| Gm609 | 1.91 | 6.28 | 2.04 | 2.25E−08 | predicted gene 609 |
| Nudt11 | 3.61 | 8.49 | 2.00 | 4.26E−10 | nudix (nucleoside |
| | | | | | diphosphate linked moiety |
| | | | | | X)-type motif 11 |
| Irx2 | 3.71 | 8.92 | 2.00 | 1.67E−09 | Iroquois related homeobox 2 |
| | | | | | (Drosophila) |
| Drc1 | 1.81 | 4.67 | 1.96 | 5.33E−08 | dynein regulatory complex |
| | | | | | subunit 1 |
| 4930539E08Rik | 1.87 | 5.64 | 1.96 | 3.36E−07 | RIKEN cDNA 4930539E08 |
| | | | | | gene |
| Olfm3 | 1.54 | 3.59 | 1.96 | 4.31E−09 | olfactomedin 3 |
| Klhl13 | 2.51 | 5.34 | 1.91 | 9.37E−11 | kelch-like 13 (Drosophila) |
| 1700086L19Rik | 59.99 | 119.24 | 1.89 | 9.75E−13 | RIKEN cDNA 1700086L19 |
| | | | | | gene |
| Smarca1 | 7.63 | 15.49 | 1.88 | 5.27E−11 | SWI/SNF related, matrix |
| | | | | | associated, actin dependent |
| | | | | | regulator of chromatin, |
| | | | | | subfamily a, member 1 |
| Dbpht2 | 14.44 | 27.59 | 1.87 | 1.92E−16 | DNA binding protein with his- |
| | | | | | thr domain |
| Mafb | 10.11 | 24.19 | 1.86 | 5.34E−07 | v-maf musculoaponeurotic |
| | | | | | fibrosarcoma oncogene |
| | | | | | family, protein B (avian) |
| Sgce | 5.07 | 10.55 | 1.84 | 2.65E−08 | sarcoglycan, epsilon |
| Gbp8 | 2.99 | 7.39 | 1.84 | 2.08E−06 | guanylate-binding protein 8 |
| Gc | 73.92 | 153.00 | 1.83 | 2.93E−09 | group specific component |
| Irx1 | 2.39 | 5.96 | 1.83 | 2.59E−06 | Iroquois related homeobox 1 |
| | | | | | (Drosophila) |
| Clec2d | 3.68 | 9.10 | 1.82 | 1.22E−06 | C-type lectin domain family |
| | | | | | 2, member d |
| Sema3e | 1.16 | 2.28 | 1.81 | 5.87E−09 | sema domain, |
| | | | | | immunoglobulin domain (Ig), |
| | | | | | short basic domain, |
| | | | | | secreted, (semaphorin) 3E |
| Dpp6 | 4.13 | 8.00 | 1.79 | 3.14E−09 | dipeptidylpeptidase 6 |
| Ffar4 | 2.66 | 5.88 | 1.78 | 2.48E−06 | Free fatty acid receptor 4 |
| Agt | 11.09 | 22.33 | 1.78 | 1.46E−07 | angiotensinogen (serpin |
| | | | | | peptidase inhibitor, clade A, |
| | | | | | member 8) |
| Nyap1 | 1.92 | 3.82 | 1.77 | 1.67E−07 | Neuronal tyrosin- |
| | | | | | phophrylated |
| | | | | | phophoinositide 3-kinase |
| | | | | | adaptor 1 |
| Klb | 2.37 | 4.59 | 1.77 | 4.06E−08 | klotho beta |
| Mamld1 | 1.95 | 5.64 | 1.75 | 2.76E−05 | mastermind-like domain |
| | | | | | containing 1 |
| Gbp9 | 1.43 | 4.03 | 1.74 | 3.43E−05 | guanylate-binding protein 9 |
| Agpat4 | 2.55 | 4.89 | 1.73 | 2.96E−07 | 1-acylglycerol-3-phosphate |
| | | | | | O-acyltransferase 4 |
| | | | | | (lysophosphatidic acid |
| | | | | | acyltransferase, delta) |
| 9030617O03Rik | 2.98 | 5.31 | 1.72 | 8.96E−10 | RIKEN cDNA 9030617O03 |
| | | | | | gene |
| Serping1 | 39.33 | 82.99 | 1.71 | 1.56E−05 | serine (or cysteine) |
| | | | | | peptidase inhibitor, clade G, |
| | | | | | member 1 |
| Oit1 | 2.23 | 4.78 | 1.71 | 1.89E−05 | oncoprotein induced |
| | | | | | transcript 1 |
| AW551984 | 3.02 | 5.27 | 1.69 | 1.68E−09 | expressed sequence |
| | | | | | AW551984 |
| Apoa2 | 5.00 | 10.76 | 1.68 | 4.21E−05 | apolipoprotein A-II |
| 1810041L15Rik | 1.81 | 3.14 | 1.67 | 1.86E−08 | RIKEN cDNA 1810041L15 |
| | | | | | gene |
| Rragb | 19.05 | 33.15 | 1.67 | 1.28E−13 | Ras-related GTP binding B |
| Gm20554 | 1.20 | 2.33 | 1.67 | 1.68E−05 | predicted gene, 20554 |
| Fgl2 | 20.32 | 37.91 | 1.65 | 1.96E−06 | fibrinogen-like protein 2 |
| Basp1 | 5.49 | 11.31 | 1.65 | 7.39E−05 | brain abundant, membrane |
| | | | | | attached signal protein 1 |
| Rbp4 | 108.27 | 210.71 | 1.64 | 3.56E−05 | retinol binding protein 4, |
| | | | | | plasma |
| Gng2 | 4.13 | 7.35 | 1.63 | 6.21E−06 | guanine nucleotide binding |
| | | | | | protein (G protein), gamma 2 |
| Syt16 | 1.20 | 2.26 | 1.63 | 4.27E−05 | synaptotagmin XVI |
| Etv1 | 19.52 | 33.99 | 1.62 | 1.70E−06 | ets variant gene 1 |
| Ptprd | 2.69 | 4.67 | 1.62 | 5.51E−06 | protein tyrosine |
| | | | | | phosphatase, receptor type, D |
| Ang | 38.11 | 69.38 | 1.62 | 2.96E−05 | angiogenin, ribonuclease, |
| | | | | | RNase A family, 5 |
| Ehf | 5.97 | 10.90 | 1.61 | 6.25E−05 | ets homologous factor |
| Tmsb15b1 | 13.94 | 28.28 | 1.60 | 2.47E−04 | thymosin beta 15b1 |
| Eda2r | 1.08 | 1.98 | 1.59 | 2.18E−04 | ectodysplasin A2 receptor |
| Arx | 3.09 | 6.42 | 1.59 | 3.70E−04 | aristaless related homeobox |
| Cobl | 1.27 | 2.27 | 1.58 | 1.22E−04 | cordon-bleu |
| Gcnt4 | 1.91 | 3.02 | 1.58 | 2.92E−08 | glucosaminyl (N-acetyl) |
| | | | | | transferase 4, core 2 (beta- |
| | | | | | 1,6-N- |
| | | | | | acetylglucosaminyltransferase) |
| Ocrl | 22.92 | 37.04 | 1.57 | 3.38E−07 | oculocerebrorenal syndrome |
| | | | | | of Lowe |
| Jchain | 27.78 | 74.91 | 1.57 | 7.54E−04 | immunoglobulin joining chain |
| Nsg2 | 1.97 | 3.49 | 1.56 | 2.62E−04 | neuron specific gene family |
| | | | | | member 2 |
| Cnih2 | 1.06 | 1.98 | 1.55 | 5.62E−04 | cornichon homolog 2 |
| | | | | | (Drosophila) |
| Blnk | 2.01 | 3.87 | 1.55 | 7.38E−04 | B cell linker |
| Sh3bgr | 1.86 | 3.39 | 1.54 | 5.02E−04 | SH3-binding domain |
| | | | | | glutamic acid-rich protein |
| Tesc | 2.74 | 4.72 | 1.54 | 2.37E−04 | tescalcin |
| Rcn1 | 8.19 | 12.75 | 1.54 | 3.00E−07 | reticulocalbin 1 |
| Gpr119 | 8.80 | 15.35 | 1.54 | 4.66E−05 | G-protein coupled receptor |
| | | | | | 119 |
| Oxtr | 4.25 | 7.25 | 1.53 | 2.46E−04 | oxytocin receptor |
| Gfra1 | 2.10 | 3.43 | 1.53 | 9.38E−05 | glial cell line derived |
| | | | | | neurotrophic factor family |
| | | | | | receptor alpha 1 |
| Apod | 1.64 | 2.75 | 1.53 | 2.67E−04 | apolipoprotein D |
| Nefm | 3.98 | 6.35 | 1.53 | 3.04E−05 | neurofilament, medium |
| | | | | | polypeptide |
| Arg1 | 31.98 | 59.62 | 1.53 | 7.70E−04 | arginase, liver |
| Ptprg | 1.92 | 3.12 | 1.52 | 1.34E−04 | protein tyrosine |
| | | | | | phosphatase, receptor type, G |
| F5 | 1.02 | 1.71 | 1.51 | 3.56E−04 | coagulation factor V |
| Wfdc16 | 3.40 | 5.87 | 1.51 | 6.21E−04 | WAP four-disulfide core |
| | | | | | domain 16 |
| Rbms3 | 1.52 | 2.38 | 1.51 | 4.76E−05 | RNA binding motif, single |
| | | | | | stranded interacting protein |
| Pla2g2d | 3.36 | 5.69 | 1.50 | 6.42E−04 | phospholipase A2, group IID |
| Ctse | 8.26 | 1.73 | −1.50 | 8.53E−04 | cathepsin E |
| Vnn1 | 1.44 | 0.42 | −1.50 | 1.56E−03 | vanin 1 |
| Gamt | 1.61 | 0.58 | −1.50 | 2.14E−03 | guanidinoacetate |
| | | | | | methyltransferase |
| Necab2 | 12.61 | 7.29 | −1.50 | 8.21E−05 | N-terminal EF-hand calcium |
| | | | | | binding protein 2 |
| Mapk13 | 5.55 | 2.86 | −1.50 | 1.19E−03 | mitogen-activated protein |
| | | | | | kinase 13 |
| Asb11 | 2.49 | 1.00 | −1.51 | 2.20E−03 | ankyrin repeat and SOCS |
| | | | | | box-containing 11 |
| Apol7a | 1.39 | 0.28 | −1.51 | 6.83E−04 | apolipoprotein L 7a |
| Sh2d4a | 1.70 | 0.65 | −1.52 | 1.75E−03 | SH2 domain containing 4A |
| Anxa3 | 25.80 | 11.67 | −1.53 | 1.06E−03 | annexin A3 |
| Hspb1 | 19.63 | 10.32 | −1.54 | 3.56E−04 | heat shock protein 1 |
| Slc38a3 | 2.59 | 0.79 | −1.54 | 9.73E−04 | solute carrier family 38, |
| | | | | | member 3 |
| Cdc42ep1 | 4.05 | 1.77 | −1.54 | 1.15E−03 | CDC42 effector protein (Rho |
| | | | | | GTPase binding) 1 |
| Rasd1 | 21.48 | 10.12 | −1.54 | 7.87E−04 | RAS, dexamethasone- |
| | | | | | induced 1 |
| Matn2 | 4.77 | 2.36 | −1.55 | 4.97E−04 | matrilin 2 |
| Car9 | 2.36 | 0.79 | −1.55 | 8.86E−04 | carbonic anhydrase 9 |
| Kcnf1 | 1.86 | 0.86 | −1.56 | 7.21E−04 | potassium voltage-gated |
| | | | | | channel, subfamily F, |
| | | | | | member 1 |
| Nptxr | 8.64 | 4.03 | −1.57 | 4.39E−04 | neuronal pentraxin receptor |
| Tead2 | 5.32 | 2.17 | −1.60 | 3.84E−04 | TEA domain family member 2 |
| Anxa13 | 5.24 | 0.65 | −1.60 | 7.08E−05 | annexin A13 |
| Krt19 | 69.04 | 26.52 | −1.61 | 3.66E−04 | keratin 19 |
| Dmbt1 | 54.65 | 10.80 | −1.61 | 1.51E−04 | deleted in malignant brain |
| | | | | | tumors 1 |
| Ntn1 | 1.62 | 0.65 | −1.61 | 3.25E−04 | netrin 1 |
| Krt20 | 1.99 | 0.65 | −1.63 | 2.34E−04 | keratin 20 |
| 1810011O10Rik | 11.19 | 5.88 | −1.64 | 1.31E−06 | RIKEN cDNA 1810011O10 |
| | | | | | gene |
| Igsf23 | 1.93 | 0.40 | −1.65 | 9.19E−05 | immunoglobulin superfamily, |
| | | | | | member 23 |
| Npr1 | 6.75 | 3.07 | −1.65 | 5.93E−05 | natriuretic peptide receptor 1 |
| St8sia1 | 4.77 | 2.35 | −1.67 | 4.85E−06 | ST8 alpha-N-acetyl- |
| | | | | | neuraminide alpha-2,8- |
| | | | | | sialyltransferase 1 |
| Fgd3 | 2.45 | 0.83 | −1.67 | 1.21E−04 | FYVE, RhoGEF and PH |
| | | | | | domain containing 3 |
| Serpinb6b | 4.84 | 1.59 | −1.68 | 1.10E−04 | serine (or cysteine) |
| | | | | | peptidase inhibitor, clade B, |
| | | | | | member 6b |
| Kcna2 | 1.80 | 0.81 | −1.68 | 2.00E−05 | potassium voltage-gated |
| | | | | | channel, shaker-related |
| | | | | | subfamily, member 2 |
| Itgb4 | 4.36 | 1.65 | −1.69 | 7.98E−05 | integrin beta 4 |
| Clic6 | 1.68 | 0.26 | −1.69 | 2.60E−05 | chloride intracellular channel 6 |
| Lamb3 | 3.30 | 1.16 | −1.71 | 6.10E−05 | laminin, beta 3 |
| Kcnab3 | 4.81 | 2.28 | −1.73 | 4.62E−07 | potassium voltage-gated |
| | | | | | channel, shaker-related |
| | | | | | subfamily, beta member 3 |
| Spns2 | 2.30 | 0.98 | −1.76 | 4.27E−06 | spinster homolog 2 |
| Ano1 | 3.91 | 1.26 | −1.78 | 1.34E−05 | anoctamin 1, calcium |
| | | | | | activated chloride channel |
| Acta1 | 4.46 | 1.05 | −1.79 | 1.17E−05 | actin, alpha 1, skeletal |
| | | | | | muscle |
| Clu | 1486.73 | 481.69 | −1.80 | 7.21E−06 | clusterin |
| Bok | 4.05 | 1.31 | −1.85 | 3.28E−06 | BCL2-related ovarian killer |
| | | | | | protein |
| Ptf1a | 3.14 | 0.52 | −1.95 | 3.99E−07 | pancreas specific |
| | | | | | transcription factor, 1a |
| Krt23 | 11.33 | 2.17 | −1.96 | 4.29E−07 | keratin 23 |
| Tspan1 | 8.22 | 1.40 | −2.50 | 5.21E−12 | tetraspanin 1 |

*10 mg/kg Control or α-GCGR

| | Gcgr | | | | |
|---|---|---|---|---|---|
| | WT | Gcgr KO | Fold | | |
| Symbol | RPKM | RPKM | change | Padj | Description |
| Dapl1 | 2.01 | 104.79 | 26.21 | 2.36E−11 | death associated protein-like 1 |
| Cd2 | 0.18 | 18.69 | 19.23 | 4.88E−05 | CD2 antigen |
| Slc38a5 | 13.11 | 309.74 | 18.28 | 1.34E−12 | solute carrier family 38, |
| | | | | | member 5 |
| Cidea | 0.00 | 6.85 | 10.84 | 1.63E−02 | cell death-inducing DNA |
| | | | | | fragmentation factor, alpha |
| | | | | | subunit-like effector A |
| Rspo4 | 0.17 | 5.99 | 9.77 | 6.75E−03 | R-spondin family, member 4 |
| Crym | 0.24 | 8.77 | 7.93 | 4.36E−02 | crystallin, mu |
| Vgf | 6.19 | 52.26 | 7.70 | 6.79E−09 | VGF nerve growth factor |
| | | | | | inducible |
| Gca | 0.79 | 8.83 | 6.78 | 4.19E−03 | grancalcin |
| Eda2r | 0.38 | 3.92 | 5.99 | 3.86E−02 | ectodysplasin A2 receptor |
| Myh14 | 1.46 | 9.35 | 5.92 | 8.54E−06 | myosin, heavy polypeptide |
| | | | | | 14 |
| Ccng1 | 7.36 | 44.43 | 5.33 | 6.01E−06 | cyclin G1 |
| Gmds | 3.71 | 25.51 | 5.01 | 1.78E−02 | GDP-mannose 4,6- |
| | | | | | dehydratase |
| Spc25 | 15.96 | 85.13 | 4.92 | 1.14E−04 | SPC25, NDC80 kinetochore |
| | | | | | complex component, |
| | | | | | homolog (S. cerevisiae) |
| Cldn6 | 2.23 | 14.40 | 4.81 | 4.32E−02 | claudin 6 |
| Agt | 8.85 | 42.05 | 4.37 | 2.63E−04 | angiotensinogen (serpin |
| | | | | | peptidase inhibitor, clade A, |
| | | | | | member 8) |
| Eef1a2 | 12.44 | 59.89 | 4.29 | 4.75E−04 | eukaryotic translation |
| | | | | | elongation factor 1 alpha 2 |
| Slc35b1 | 7.91 | 37.03 | 3.92 | 4.32E−02 | solute carrier family 35, |
| | | | | | member B1 |
| Atpif1 | 28.02 | 119.68 | 3.79 | 5.59E−03 | ATPase inhibitory factor 1 |
| Scg3 | 74.79 | 294.63 | 3.72 | 2.85E−05 | secretogranin III |
| Oxtr | 9.52 | 34.57 | 3.48 | 9.73E−05 | oxytocin receptor |
| Foxn2 | 1.54 | 6.10 | 3.42 | 4.90E−02 | forkhead box N2 |
| Pik3r3 | 5.02 | 17.92 | 3.36 | 1.36E−03 | phosphatidylinositol 3 |
| | | | | | kinase, regulatory subunit, |
| | | | | | polypeptide 3 (p55) |
| Ppa1 | 9.23 | 32.13 | 3.31 | 4.65E−02 | pyrophosphatase (inorganic) 1 |
| Manf | 14.93 | 49.89 | 3.12 | 3.82E−02 | mesencephalic astrocyte- |
| | | | | | derived neurotrophic factor |
| Ifih1 | 8.46 | 27.43 | 3.10 | 4.19E−03 | interferon induced with |
| | | | | | helicase C domain 1 |
| Ssr4 | 112.41 | 372.40 | 3.09 | 1.80E−02 | signal sequence receptor, |
| | | | | | delta |
| Tmem27 | 440.82 | 1385.10 | 3.05 | 1.17E−05 | transmembrane protein 27 |
| Sec11c | 26.57 | 87.79 | 3.04 | 2.38E−02 | SEC11 homolog C (S. cerevisiae) |
| 1700086L19Rik | 61.92 | 200.58 | 3.03 | 3.13E−02 | RIKEN cDNA 1700086L19 |
| | | | | | gene |
| Rragb | 15.42 | 48.21 | 2.96 | 1.22E−02 | Ras-related GTP binding B |
| Maged2 | 22.95 | 73.46 | 2.95 | 3.38E−02 | melanoma antigen, family D, 2 |
| Ttr | 1872.39 | 5567.92 | 2.88 | 2.63E−04 | transthyretin |
| Gpx3 | 183.77 | 529.49 | 2.76 | 6.90E−03 | glutathione peroxidase 3 |
| Atp6ap2 | 24.49 | 69.75 | 2.69 | 2.42E−02 | ATPase, H+ transporting, |
| | | | | | lysosomal accessory protein 2 |
| Higd1a | 18.31 | 52.01 | 2.68 | 4.32E−02 | HIG1 domain family, |
| | | | | | member 1A |
| Tmed3 | 106.88 | 297.42 | 2.67 | 2.38E−02 | transmembrane emp24 |
| | | | | | domain containing 3 |
| Dynlt1a | 51.58 | 145.20 | 2.63 | 4.36E−02 | dynein light chain Tctex-type |
| | | | | | 1A |
| Gcg | 35771.85 | 96500.33 | 2.63 | 6.39E−04 | glucagon |
| Dynlt1b | 57.10 | 158.58 | 2.62 | 3.79E−02 | dynein light chain Tctex-type |
| | | | | | 1B |
| Chga | 1110.12 | 2978.38 | 2.60 | 3.67E−03 | chromogranin A |
| Gc | 86.86 | 228.48 | 2.54 | 9.28E−03 | group specific component |
| Hap1 | 23.62 | 56.91 | 2.32 | 3.75E−02 | huntingtin-associated protein 1 |
| Scg2 | 160.15 | 383.81 | 2.31 | 4.36E−02 | secretogranin II |
| Sepp1 | 108.03 | 254.40 | 2.30 | 7.88E−03 | selenoprotein P, plasma, 1 |
| Ccnd2 | 64.36 | 149.51 | 2.26 | 3.13E−02 | cyclin D2 |
| Cpe | 527.04 | 1056.35 | 1.97 | 4.93E−02 | carboxypeptidase E |
| Fos | 229.65 | 97.22 | −2.29 | 4.93E−02 | FBJ osteosarcoma |
| | | | | | oncogene |
| Abca1 | 25.09 | 10.17 | −2.37 | 4.64E−02 | ATP-binding cassette, sub- |
| | | | | | family A (ABC1), member 1 |
| Ndrg1 | 25.23 | 8.09 | −2.85 | 4.91E−02 | N-myc downstream |
| | | | | | regulated gene 1 |
| Ptprb | 17.88 | 5.51 | −3.05 | 1.77E−02 | protein tyrosine |
| | | | | | phosphatase, receptor type, B |
| Tenc1 | 14.19 | 4.18 | −3.09 | 4.90E−02 | tensin like C1 domain- |
| | | | | | containing phosphatase |
| Igf1r | 4.06 | 1.21 | −3.15 | 3.96E−02 | insulin-like growth factor I |
| | | | | | receptor |
| Fosb | 83.14 | 24.63 | −3.22 | 1.33E−03 | FBJ osteosarcoma |
| | | | | | oncogene B |
| Lrp1 | 15.93 | 4.34 | −3.30 | 4.32E−02 | low density lipoprotein |
| | | | | | receptor-related protein 1 |
| ler2 | 98.41 | 23.44 | −3.68 | 4.36E−02 | immediate early response 2 |
| Kalrn | 2.64 | 0.64 | −3.71 | 4.32E−02 | kalirin, RhoGEF kinase |
| Npas4 | 20.85 | 4.82 | −3.88 | 1.96E−03 | neuronal PAS domain |
| | | | | | protein 4 |
| Ankrd44 | 11.33 | 2.39 | −4.17 | 1.15E−03 | ankyrin repeat domain 44 |
| Tmem222 | 16.81 | 2.51 | −4.87 | 3.79E−02 | transmembrane protein 222 |
| Fgb | 21.18 | 3.80 | −4.96 | 4.39E−03 | fibrinogen beta chain |
| Ltbp4 | 15.96 | 2.09 | −5.37 | 4.65E−02 | latent transforming growth |
| | | | | | factor beta binding protein 4 |
| Mt2 | 74.20 | 10.43 | −5.39 | 4.36E−02 | metallothionein 2 |
| Mt1 | 165.84 | 19.89 | −6.15 | 1.40E−02 | metallothionein 1 |
| Nid2 | 6.62 | 0.72 | −6.47 | 2.21E−02 | nidogen 2 |
| Plce1 | 2.78 | 0.25 | −6.98 | 2.04E−02 | phospholipase C, epsilon 1 |
| Camk1d | 2.07 | 0.14 | −7.78 | 1.63E−02 | calcium/calmodulin- |
| | | | | | dependent protein kinase ID |
| Klrb1b | 3.77 | 0.09 | −8.37 | 4.37E−02 | killer cell lectin-like receptor |
| | | | | | subfamily B member 1B |
| Il10ra | 4.03 | 0.14 | −8.41 | 2.22E−02 | interleukin 10 receptor, alpha |
| Rimklb | 1.85 | 0.00 | −8.87 | 4.61E−02 | ribosomal modification |
| | | | | | protein rimK-like family |
| | | | | | member B |
| Nlrp3 | 3.49 | 0.18 | −8.97 | 1.97E−02 | NLR family, pyrin domain |
| | | | | | containing 3 |
| Baiap2 | 2.71 | 0.12 | −9.08 | 1.79E−02 | brain-specific angiogenesis |
| | | | | | inhibitor 1-associated protein 2 |
| Daam2 | 3.14 | 0.10 | −9.97 | 6.80E−03 | dishevelled associated |
| | | | | | activator of morphogenesis 2 |
| Adamts15 | 2.41 | 0.07 | −10.62 | 7.14E−03 | a disintegrin-like and |
| | | | | | metallopeptidase (reprolysin |
| | | | | | type) with thrombospondin |
| | | | | | type 1 motif, 15 |
| Mamdc2 | 2.95 | 0.00 | −12.78 | 5.93E−03 | MAM domain containing 2 |
| Epha7 | 1.34 | 0.00 | −16.19 | 8.25E−04 | Eph receptor A7 |
| Adamts2 | 4.59 | 0.00 | −21.20 | 7.88E−05 | a disintegrin-like and |
| | | | | | metallopeptidase (reprolysin |
| | | | | | type) with thrombospondin |
| | | | | | type 1 motif, 2 |

*15 mg/kg Control or α-GCGR

### Slc38a5 is Required for α-Cell Proliferation Following Inhibition of GCGR Signaling

Ki67 staining was used to detect cell proliferation in pancreatic islets from GCGR-antibody treated mice. Cell proliferation was negligible in islets from control antibody treated mice and in non-Gcg(+) islet cells from GCGR-antibody treated mice (FIG. 3A). However, Ki67 staining was detected in GCG(+) cells from the GCGR-antibody treated mice (FIG. 3A). FIG. 3B shows a pair of α-cells that have undergone cell division and stain positive for Slc38a5 and Ki67. Interestingly, the proliferation rate was 4-times greater in Slc38a5(+) than in Slc38a5(−) α-cells (FIG. 3C).

Next, αTC1-6 glucagonoma cells were generated with reduced expression of Slc38a5 to examine the role of this amino acid transporter in cell division. CRISPR-Cas9 was used to delete the first exon of Slc38a5 (FIG. 8A). This resulted in reduced size of detected cDNA (FIG. 8B), lower mRNA levels of the transcript detected by Taqman (FIG. 8C) or RNAseq (FIG. 8D) and protein expression (FIG. 8E). αTC1-6 cells with reduced expression of Slc38a5 showed 50% lower rate of cell proliferation when compared to wildtype αTC1-6 cells (FIG. 3D). Supplementing the cell culture medium with the Slc38a5 substrates glutamine or alanine (4 mM) significantly increased the rate of cell proliferation (FIG. 3D). Interestingly, glutamine and alanine also increased cell proliferation in mutant cell line but to a much lesser extent than in the wildtype cells (FIG. 3D). These data show that Slc38a5 is required for amino acid induced α-cell proliferation.

mTOR-Dependent α-Cell Hyperplasia in GCGR Antibody Treated Mice

We treated mice with GCGR or control antibody (3 mg/kg) for 21 days in combination with PBS or the mTOR inhibitor rapamycin (10 mg/kg, daily). Rapamycin did not affect the ability of the GCGR-antibody to reduce blood glucose levels (FIG. 4A). Body and pancreas weights did not change in the treatment groups (FIGS. 9A and 9B). Consistent with our previous data (Okamoto et al., 2015), plasma amino acid levels were significantly increased in GCGR-antibody treated mice (FIG. 4B). Rapamycin did not affect plasma amino acid levels in control antibody treated mice but further increased levels in mice receiving the GCGR-antibody. The higher plasma amino acid levels in GCGR-antibody and rapamycin-treated mice might result from a small reduction in muscle mass (FIG. 9C). Rapamycin did not affect hyperglucagonemia in GCGR-antibody treated mice (FIG. 4C). α-cell mass was increased 3-fold in GCGR-antibody treated mice and the increase was effectively blocked by rapamycin (FIGS. 4D and 4E). Masses of α-, β- and PP cells were unchanged by treatments (FIGS. 9D-9F). Plasma insulin levels were unchanged in the treatment groups (FIG. 4F). α-cell size was slightly increased by the GCGR-antibody. Rapamycin reduced cell size equally in control and GCGR-antibody treated mice (FIG. 4G). Interestingly, the increase in Slc38a5 mRNA expression induced by GCGR-antibody was blocked by rapamycin (FIG. 4H). As a consequence, only a few α-cells expressing Slc38a5 were detected in mice receiving GCGR-antibody and rapamycin (FIGS. 4D and 41). These data show that GCGR antibody-mediated increase in α-cell mass and Slc38a5 expression is mTOR-dependent and blocked by rapamycin.

Reduced α-Cell Hyperplasia in Slc38a5^{-/-} Mice Treated with GCGR-Antibody

Slc38a5^{-/-} mice have no gross abnormalities and are born with the expected Mendelian ratio. The mice have normal body, pancreas and liver weights (FIGS. 10A-10C), blood glucose levels (FIG. 5A), plasma levels of amino acids, glucagon and insulin (FIGS. 5B-5D), oral glucose and insulin tolerance tests (FIGS. 10D and 10E) and α- and β-cell masses, α-cell size and number of islets per pancreas area (FIGS. 5E-5I). Treatment of Slc38a5^{-/-} mice with the GCGR-antibody reduced blood glucose levels to the same extent as in littermate control mice (FIG. 5A). Similar changes were observed in plasma amino acid and glucagon levels as well as in pancreas weights between Slc38a5^{-/-} and control mice treated with GCGR-antibody (FIGS. 5B, 5C and 10B). No effects were observed on plasma insulin levels (FIG. 5D). While Slc38a5^{-/-} mice and control mice exhibited similar hyperaminoacidemia and hyperglucagonemia following glucagon blockade, the expansion of α-cell mass following GCGR-antibody inhibition was reduced by 50% in Slc38a5^{-/-} mice relative to treated control mice (FIG. 5F), without affecting changes in α-cell size (FIG. 5G). GCGR antibody did not affect α-cell mass or number of islets per pancreas area (FIGS. 5H and 5I). These data show that Slc38a5 is required for GCGR inhibition-induced α-cell hyperplasia, and it represents a prominent amino acid transporter in mediating amino acid stimulated α-cell proliferation both cultured α-cells (FIG. 3D) and in vivo.

### SUMMARY

This study demonstrated that inhibition of glucagon signaling with a monoclonal blocking antibody to the glucagon receptor lowered blood glucose levels and resulted in reduced hepatic expression of genes involved in the uptake and metabolism of amino acids and gluconeogenesis, elevated plasma amino acid levels, and expanded alpha-cell mass via an mTOR-dependent pathway. Blocking the glucagon pathway also increased expression of the amino acid transporter Slc38a5 in a subset of highly proliferative pancreatic alpha-cells, and this transporter was selectively and prominently involved in mediating the alpha-cell hyperplasia triggered by hyperaminoacidemia; most importantly, mice deficient in Slc38a5 had reduced alpha-cell mass following treatment with the GCGR-antibody. These data demonstrate that amino acids and the amino acid transporter Slc38a5 are key components in the feedback loop between glucagon receptor signaling in the liver and compensatory changes in circulating glucagon levels and alpha-cell mass to ensure sufficient capacity and robustness of this circuit to maintain normal blood glucose levels.

Alpha-cells express many amino acid transporters; inhibition of glucagon signaling selectively increased the expression of Slc38a5. The expression of Slc38a5 was restricted to proliferating alpha-cells and was not detected in the other islet cell types. The increase in alpha-cell mass following GCGR inhibition was blocked by rapamycin.

Interestingly, the expression of Slc38a5 was also blocked by rapamycin. This suggests that mTOR regulates the expression of Slc38a5 in a feed forward mechanism where uptake of amino acids by the alpha-cells activates mTOR, which triggers the expression of Slc38a5, further enhancing amino acid uptake, mTOR activation and Slc38a5 expression, culminating in cell division. It is important to note that rapamycin did not block the GCGR inhibition-induced increase in plasma glucagon levels.

The data suggest that Slc38a5 is important for amino acid-induced alpha-cell proliferation and expansion of alpha-cell mass following GCGR inhibition, but not for formation and maintenance of alpha-cell mass. This is supported by the finding that Slc38a5^{-/-} mice have normal alpha-cell mass. In addition, αTC1-6 glucagonoma cells express Slc38a5 and knockdown of this amino acid transporter strongly reduced their proliferative capacity in response to glutamine or alanine. These data suggest that inhibition of Slc38a5 could be used therapeutically to reduce alpha-cell hyperplasia following GCGR inhibition, which is a potential safety concern in settings of chronic use of agents that reduce glucagon receptor signaling for treating diabetes.

What is claimed is:

1. A method for lowering blood glucose levels, or for treating a condition or disease associated with, or characterized in part by high blood glucose levels, or at least one symptom or complication associated with the condition or disease, the method comprising administering a therapeutically effective amount of a glucagon signaling pathway antagonist in combination with a therapeutically effective amount of an inhibitor of the amino acid transporter Solute Carrier Family 38 Member 5 (SLC38A5), to a patient in need thereof, such that blood glucose levels are lowered or that the condition or disease is mediated, or at least one symptom or complication associated with the condition or disease is alleviated or reduced in severity.

2. The method of claim 1, wherein the condition or disease is selected from the group consisting of diabetes, impaired glucose tolerance, obesity, nephropathy, neuropathy, retinopathy, cataracts, stroke, atherosclerosis, impaired wound healing, diabetic ketoacidosis, hyperglycemia, hyperglycemic hyperosmolar syndrome, perioperative hyperglycemia, hyperglycemia in the intensive care unit patient, hyperinsulinemia, the metabolic syndrome, insulin resistance syndrome and impaired fasting glucose.

3. The method of claim 1, wherein the glucagon signaling pathway antagonist is a glucagon (GCG) inhibitor or a glucagon receptor (GCGR) antagonist.

4. The method of claim 3, wherein the GCG inhibitor or GCGR antagonist is selected from the group consisting of antisense molecules, GCGR antibodies, small molecule inhibitors, shRNA, siRNA, peptide inhibitors, DARPins, Spiegelmers, aptamers, engineered Fn type-III domains, GCG antibodies, and derivatives thereof.

5. The method of claim 3, wherein the GCG inhibitor or GCGR antagonist is an isolated human monoclonal antibody, or an antigen binding fragment thereof.

6. The method of claim 5, wherein the GCGR antagonist is an isolated human monoclonal antibody or antigen-binding fragment thereof comprising the complementarity determining regions (CDRs) of a heavy chain variable region (HCVR), wherein the HCVR has an amino acid sequence selected from the group consisting of SEQ ID NOs: 2, 18, 34, 50, 66, 70, 86, 90, 106, 110, 126, 130 and 146; and the CDRs of a light chain variable region (LCVR), wherein the LCVR has an amino acid sequence selected from the group consisting of SEQ ID NOs: 10, 26, 42, 58, 68, 78, 88, 98, 108, 118, 128, 138 and 148.

7. The method of claim 6, wherein the isolated antibody or antigen-binding fragment thereof comprises: (a) a HCVR having an amino acid sequence selected from the group consisting of SEQ ID NOs: 2, 18, 34, 50, 66, 70, 86, 90, 106, 110, 126, 130 and 146; and/or (b) a LCVR having an amino acid sequence selected from the group consisting of SEQ ID NOs: 10, 26, 42, 58, 68, 78, 88, 98, 108, 118, 128, 138 and 148.

8. The method of claim 6, wherein the isolated antibody or antigen-binding fragment thereof comprises a HCVR/LCVR sequence pair selected from the group consisting of SEQ ID NOs: 2/10, 18/26, 34/42, 50/58, 66/68, 70/78, 86/88, 90/98, 106/108, 110/118, 126/128, 130/138, and 146/148.

9. The method of claim 6, wherein the isolated antibody or antigen-binding fragment thereof comprises a HCVR/LCVR amino acid sequence pair as set forth in SEQ ID NOs: 86/88.

10. The method of claim 5, wherein the GCG inhibitor is an isolated human monoclonal antibody or antigen-binding fragment thereof comprising: (a) three heavy chain complementarity determining regions (HCDR1, HCDR2 and HCDR3) contained within a heavy chain variable region (HCVR) amino acid sequence selected from the group consisting of SEQ ID NOs: 150, 166, 182, 198, 214, 230, 246, 262, 278, and 294; and (b) three light chain CDRs (LCDR1, LCDR2 and LCDR3) contained within a light chain variable region (LCVR) amino acid sequence selected from the group consisting of SEQ ID NOs: 158, 174, 190, 206, 222, 238, 254, 270, 286, and 302.

11. The method of claim 10, wherein the isolated antibody or antigen binding fragment thereof comprises an HCVR having an amino acid sequence selected from the group consisting of SEQ ID NOs: 150, 166, 182, 198, 214, 230, 246, 262, 278, and 294 and/or a LCVR having an amino acid sequence selected from the group consisting of SEQ ID NOs: 158, 174, 190, 206, 222, 238, 254, 270, 286, and 302.

12. The method of claim 10, wherein the isolated antibody or antigen-binding fragment thereof comprises a HCVR/LCVR amino acid sequence pair selected from the group consisting of SEQ ID NOs: 150/158, 166/174, 182/190, 198/206, 214/222, 230/238, 246/254, 262/270, 278/286, and 294/302.

13. The method of claim 10, wherein the isolated antibody or antigen-binding fragment thereof comprises the HCVR/LCVR amino acid sequence pair of SEQ ID NOs: 166/174 or SEQ ID NOs: 182/190.

14. The method of claim 1, wherein the isolated antibody or antigen-binding fragment thereof competes for specific binding to or binds the same epitope as human GCGR with an antibody or antigen-binding fragment according to claim 6.

15. The method of claim 1, wherein the isolated antibody or antigen-binding fragment thereof competes for binding to or binds the same epitope on human GCG as an antibody or antigen-binding fragment according to claim 10.

16. The method of claim 1, wherein the inhibitor of SLC38A5 is selected from the group consisting of a small organic molecule, a protein, a polypeptide, an antibody, an siRNA and an antisense molecule.

17. The method of claim 5, wherein the isolated monoclonal antibody, or an antigen-binding fragment thereof that binds specifically to human GCGR or human GCG is administered subcutaneously, intravenously or intramuscularly.

18. The method of claim 1, wherein the SLC38A5 inhibitor is administered orally, subcutaneously, intravenously or intramuscularly.

19. The method of claim 1, wherein the glucagon signaling pathway antagonist and the SLC38A5 inhibitor are administered concurrently or sequentially.

20. The method of claim 1, wherein the glucagon signaling pathway antagonist and the SLC38A5 inhibitor are administered at therapeutically effective concentrations in separate pharmaceutical compositions or are co-formulated in one pharmaceutical composition.

21. The method of claim 1, further comprising administration of one or more therapeutic agents.

22. The method of claim 21, wherein the one or more therapeutic agents are selected from the group consisting of insulin, a biguanide (metformin), a sulfonylurea (such as glyburide, glipizide), a PPAR gamma agonist (pioglitazone, rosiglitazone), an alpha glucosidase inhibitor (acarbose, voglibose), EXENATIDE® (glucagon-like peptide 1), SYMLIN® (pramlintide), a glucagon antagonist, and a second GCGR antagonist.

23. The method of claim 21, wherein the one or more therapeutic agents is a 3-hydroxy-3-methyl-glutaryl-CoA reductase (HMG-CoA reductase) inhibitor.

24. The method of claim 23, wherein the HMG-CoA reductase inhibitor is a statin selected from the group consisting of atorvastatin, fluvastatin, lovastatin, pitavastatin, pravastatin, rosuvastatin and simvastatin.

25. A method for lowering blood glucose levels, or for treating a condition or disease associated with, or characterized in part by high blood glucose levels, or at least one symptom or complication associated with the condition or disease, the method comprising administering a therapeutically effective amount of a glucagon signaling pathway antagonist in combination with a therapeutically effective amount of an inhibitor of mechanistic target of rapamycin (mTOR) to a patient in need thereof, such that blood glucose levels are lowered or that the condition or disease is mediated, or at least one symptom or complication associated with the condition or disease is alleviated or reduced in severity.

26. The method of claim 25, wherein the condition or disease is selected from the group consisting of diabetes, impaired glucose tolerance, obesity, nephropathy, neuropathy, retinopathy, cataracts, stroke, atherosclerosis, impaired wound healing, diabetic ketoacidosis, hyperglycemia, hyperglycemic hyperosmolar syndrome, perioperative hyperglycemia, hyperglycemia in the intensive care unit patient, hyperinsulinemia, the metabolic syndrome, insulin resistance syndrome and impaired fasting glucose.

27. The method of claim 25, wherein the glucagon signaling pathway antagonist is a GCG inhibitor or a glucagon receptor GCGR antagonist.

28. The method of claim 27, wherein the GCG inhibitor or GCGR antagonist is selected from the group consisting of antisense molecules, GCGR antibodies, small molecule inhibitors, shRNA, siRNA, peptide inhibitors, DARPins, Spiegelmers, aptamers, engineered Fn type-III domains, GCG antibodies, and derivatives thereof.

29. The method of claim 27, wherein the GCG inhibitor or GCGR antagonist is an isolated human monoclonal antibody, or an antigen binding fragment thereof.

30. The method of claim 29, wherein the GCGR antagonist is an isolated human monoclonal antibody or antigen-binding fragment thereof comprising the complementarity determining regions (CDRs) of a heavy chain variable region (HCVR), wherein the HCVR has an amino acid sequence selected from the group consisting of SEQ ID NOs: 2, 18, 34, 50, 66, 70, 86, 90, 106, 110, 126, 130 and 146; and the CDRs of a light chain variable region (LCVR), wherein the LCVR has an amino acid sequence selected from the group consisting of SEQ ID NOs: 10, 26, 42, 58, 68, 78, 88, 98, 108, 118, 128, 138 and 148.

31. The method of claim 30, wherein the isolated antibody or antigen-binding fragment thereof comprises: (a) a HCVR having an amino acid sequence selected from the group consisting of SEQ ID NOs: 2, 18, 34, 50, 66, 70, 86, 90, 106, 110, 126 , 130 and 146; and/or (b) a LCVR having an amino acid sequence selected from the group consisting of SEQ ID NOs: 10, 26, 42, 58, 68, 78, 88, 98, 108, 118, 128, 138 and 148.

32. The method of claim 30, wherein the isolated antibody or antigen-binding fragment thereof comprises a HCVR/LCVR sequence pair selected from the group consisting of SEQ ID NOs: 2/10, 18/26, 34/42, 50/58, 66/68, 70/78, 86/88, 90/98, 106/108, 110/118, 126/128, 130/138, and 146/148.

33. The method of claim 30, wherein the isolated antibody or antigen-binding fragment thereof comprises a HCVR/LCVR amino acid sequence pair as set forth in SEQ ID NOs: 86/88.

34. The method of claim 29, wherein the GCG inhibitor is an isolated human monoclonal antibody or antigen-binding fragment thereof comprising: (a) three heavy chain complementarity determining regions (HCDR1, HCDR2 and HCDR3) contained within a heavy chain variable region (HCVR) amino acid sequence selected from the group consisting of SEQ ID NOs: 150, 166, 182, 198, 214, 230, 246, 262, 278, and 294; and (b) three light chain CDRs (LCDR1, LCDR2 and LCDR3) contained within a light chain variable region (LCVR) amino acid sequence selected from the group consisting of SEQ ID NOs: 158, 174, 190, 206, 222, 238, 254, 270, 286, and 302.

35. The method of claim 34, wherein the isolated antibody or antigen binding fragment thereof comprises an HCVR having an amino acid sequence selected from the group consisting of SEQ ID NOs: 150, 166, 182, 198, 214, 230, 246, 262, 278, and 294 and/or a LCVR having an amino acid sequence selected from the group consisting of SEQ ID NOs: 158, 174, 190, 206, 222, 238, 254, 270, 286, and 302.

36. The method of claim 34, wherein the isolated antibody or antigen-binding fragment thereof comprises a HCVR/LCVR amino acid sequence pair selected from the group consisting of SEQ ID NOs: 150/158, 166/174, 182/190, 198/206, 214/222, 230/238, 246/254, 262/270, 278/286, and 294/302.

37. The method of claim 34, wherein the isolated antibody or antigen-binding fragment thereof comprises the HCVR/LCVR amino acid sequence pair of SEQ ID NOs: 166/174 or SEQ ID NOs: 182/190.

38. The method of claim 25, wherein the isolated antibody or antigen-binding fragment thereof competes for specific binding to or binds the same epitope as human GCGR with an antibody or antigen-binding fragment according to claim 30.

39. The method of claim 25, wherein the isolated antibody or antigen-binding fragment thereof competes for specific binding to or binds the same epitope on human GCG as an antibody or antigen-binding fragment according to claim 34.

40. The method of claim 25, wherein the inhibitor of mTOR is selected from the group consisting of a small organic molecule, a protein, a polypeptide, an antibody, an siRNA and an antisense molecule.

41. The method of claim 25, wherein the isolated monoclonal antibody, or an antigen-binding fragment thereof that binds specifically to human GCGR or human GCG is administered subcutaneously, intravenously or intramuscularly.

42. The method of claim 25, wherein the mTOR inhibitor is administered orally, subcutaneously, intravenously or intramuscularly.

43. The method of claim 25, wherein the GCGR antagonist and the mTOR inhibitor are administered concurrently or sequentially.

44. The method of claim 25, wherein the GCGR antagonist and the mTOR inhibitor are administered at therapeutically effective concentrations in separate pharmaceutical compositions or are co-formulated in one pharmaceutical composition.

45. The method of claim 25, further comprising administration of one or more therapeutic agents.

46. The method of claim 45, wherein the one or more therapeutic agents are selected from the group consisting of insulin, a biguanide (metformin), a sulfonylurea (such as glyburide, glipizide), a PPAR gamma agonist (pioglitazone, rosiglitazone), an alpha glucosidase inhibitor (acarbose, voglibose), EXENATIDE® (glucagon-like peptide 1), SYMLIN® (pramlintide), a glucagon antagonist, and a second GCGR antagonist.

47. The method of claim 45, wherein the one or more therapeutic agents is a 3-hydroxy-3-methyl-glutaryl-CoA reductase (HMG-CoA reductase) inhibitor.

48. The method of claim 47, wherein the HMG-CoA reductase inhibitor is a statin selected from the group consisting of atorvastatin, fluvastatin, lovastatin, pitavastatin, pravastatin, rosuvastatin and simvastatin.

49. The method of claim 1, wherein the method for lowering blood glucose levels, or for treating a condition or disease associated with, or characterized in part, by high blood glucose levels, results in a reduction in blood glucose levels without demonstrating an increase in alpha cell hyperplasia.
